(19) United States
(12) Patent Application Publication
(10) Pub. No.: US 2025/0188421 A1
(43) Pub. Date: Jun. 12, 2025

(54) METHODS OF MAKING INDUCED PLURIPOTENT STEM CELLS

(71) Applicant: Pluristyx, Inc., Seattle, WA (US)

(72) Inventors: Kambiz Mousavi, Clarksville, MD (US); Raluca Marcu, Seattle, WA (US); Benjamin Fryer, Seattle, WA (US); Andreea Reilly, Seattle, WA (US)

(73) Assignee: Pluristyx, Inc., Seattle, WA (US)

(21) Appl. No.: 18/840,727

(22) PCT Filed: Feb. 22, 2023
(86) PCT No.: PCT/US2023/063055

§ 371 Date: Aug. 22, 2024

(51) Int. Cl. C12N 5/074; C12N 15/67

(52) U.S. Cl. CPC C12N 5/0696; C12N 15/67; C12N 2310/531; C12N 2501/125; C12N 2501/145; C12N 2501/2303; C12N 2501/2306; C12N 2501/26; C12N 2501/602; C12N 2501/603; C12N 2501/604; C12N 2501/606; C12N 2501/608; C12N 2506/1307

(57) ABSTRACT

The present disclosure relates to expressing one or more reprogramming factors utilizing translational enhancing elements in the 5′ and/or 3′ untranslated region (UTR) of synthetic mRNA. The 5′ UTRs include a mini-enhancer sequence and a Kozak sequence whereas 3′ UTR includes a spacer, a stem loop structure, and optionally, a polyadenine tail. The artificial 5′ and 3′ UTRs drive reprogramming factor expression, and in certain examples, do not include modified nucleosides, microRNA sites, or immune-evading factors.

### CROSS-REFERENCE TO RELATED APPLICATIONS

This application is a U.S. National Phase Patent Application based on International Patent Application No. PCT/US2023/063055, filed on Feb. 22, 2023, which claims priority to U.S. Provisional Patent Application No. 63/371,927 filed Aug. 19, 2022 and U.S. Provisional Patent Application No. 63/312,791 filed Feb. 22, 2022, the contents each of which are incorporated herein by reference in their entirety as if fully set forth herein.

### REFERENCE TO SEQUENCE LISTING

The Sequence Listing associated with this application is provided in XML format in lieu of a paper copy and is hereby incorporated by reference into the specification. The name of the XML file containing the Sequence Listing is 3A44540.XML. The XML file is 132,412 bytes, was created on Aug. 12, 2024, and is being submitted electronically via Patent Center.

### FIELD OF THE DISCLOSURE

The current disclosure provides engineered expression constructs having artificial 5′ and/or 3′ untranslated regions (UTRs) flanking a reprogramming factor coding sequence. The 5′ UTRs include a promoter, mini-enhancer sequence, and a Kozak sequence whereas the 3′ UTR includes a spacer, a stem loop structure, and optionally, a polyadenine tail. The artificial 5′ and 3′ UTRs increase reprogramming factor protein expression, and in certain examples, do not include modified nucleosides, microRNA sites, or immune-evading factors. The current disclosure also provides methods of producing induced pluripotent stem cells using the engineered expression constructs described herein.

### BACKGROUND OF THE DISCLOSURE

Stems cells can be an important precursor to generate or regenerate organs, repair tissues, prepare or deliver certain biological factors, or treat diseases or disorders. Presently, there are two sources of pluripotent stem cells: embryonic stem cells and induced pluripotent stem cells. Induced pluripotent stem cells (iPSC) are a type of stem cell that is derived from somatic cells and have been dedifferentiated back to pluripotency (also referred to as “reprogrammed”). Methods to reprogram the cells often rely on exogenously introduced nucleic acids (e.g., DNA or RNA) to produce factors necessary to stimulate reprogramming. The use of DNA, however, has several downsides in these methods. For example, exogenously introduced DNA can integrate into a host cell's genomic DNA, which can result in alterations and/or damage to the host cell's genomic DNA or can result in the exogenous DNA being inherited by daughter cells (whether or not the exogenous DNA has integrated into the chromosome) or by offspring. Even without integration, several steps must occur before the protein encoded by the DNA is produced. First, DNA must enter the nucleus where it can be transcribed into RNA, the transcribed RNA must then travel to the cytoplasm where it is translated into a protein. This creates substantial lag times before creation of a functional protein with each step representing an opportunity for error and damage to the cell. Furthermore, the transfected DNA may not be expressed, may not be expressed at reasonable rates, or may not be expressed at necessary concentrations. The suboptimal level of protein expression can be a distinct problem when DNA is introduced into the host cell.

Due to the important therapeutic potential of iPS cells (also referred to as iPSC), there are efforts to develop reprogramming methods that do not involve DNA. The first “non-inheriting” approaches to achieve success, including protein transduction, plasmid transfection, and the use of adenoviral vectors, were limited in application due to low efficiencies of iPSC conversion. More recently, techniques employing synthetic messenger RNA (mRNA) have been shown to generate iPSC that are integration and footprint-free. The use of mRNA in reversible gene therapy is advantageous because of its transient expression and non-transforming characteristics. mRNA, for example, does not need to enter the nucleus for expression and cannot integrate into the host genome, thereby limiting the risk of oncogenesis. Transfection rates greater than 90% can be attained with mRNA, obviating a need for selection of transfected cells and the amount of protein expressed corresponds to physiological expression.

Current protocols for mRNA-based reprogramming are labor-intensive, though, because cells need to be transfected with reprogramming factors daily for 2 weeks to induce pluripotency in human cells. These procedures also often rely on feeder cells, adding complexity, technical variability, and introducing a potential source of contamination with non-human-derived (“xeno”) biological material added to the process.

Despite the significant recent developments for using mRNA to induce pluripotency, there remains a need in the art to produce pluripotent stem cells that do not require frequent transfection or feeder cells. Consequently, there remains a significant need for a simple and highly reproducible, non-DNA based approach to generate iPSC.

### SUMMARY OF THE DISCLOSURE

An object of the present disclosure is to produce induced pluripotent stem cells (iPSC) using an engineered ribonucleic acid (e.g., mRNA) that increases the expression level of a reprogramming factor (RF).

To this end, the current disclosure provides that certain engineered expression constructs (EEC) are utilized to increase the expression level of at least one RF (e.g., at least one of Octamer-binding transcription factor 4 (Oct4), SRY-box transcription factor (Sox), Krüppel-like factor 4 (Klf), Nanog, Lin28, Myc, or SV40 large T antigen (SV40Tag)) to produce iPSC. The structural features of the EEC are useful for optimizing translation of the RF while retaining its structural and functional integrity; overcoming a threshold of expression, improving expression rates, half-life, and/or protein concentrations; optimizing protein localization; and avoiding deleterious intracellular responses such as the immune response and/or degradation pathways.

In particular embodiments, iPSC are created from somatic cells using the EEC disclosed herein, wherein the EEC includes a coding sequence that encodes at least one RF and engineered sequences for the 5′ UTR and/or 3′ UTR. In particular embodiments, RF include: Oct4, Sox, Klf, Nanog, Myc or SV40Tag, or Lin28. The present disclosure shows the use of the EEC to increase the expression of the at least one RF and therefore facilitate the reprogramming of somatic cells into iPSC. Moreover, as disclosed herein, reprogramming using the EEC is shown to work in different cell types, including fibroblasts, hematopoietic stem cells, and mesenchymal stem cells, irrespective of the manner of transfection.

The present disclosure provides methods for reprogramming somatic cells into iPSC, the methods including: contacting (transfecting) somatic cells with at least one EEC, wherein the EEC includes a (i) coding sequence encoding at least one RF (e.g., Oct4, Sox, Klf, Nanog, Lin28, Myc, or SV40Tag) operably linked to (ii) an engineered 5′ UTR and/or an engineered 3′ UTR.

In particular embodiments, the engineered 5′ UTR includes a promoter, a Kozak sequence, and a mini-enhancer sequence. In particular embodiments, the promoter includes a minimal promoter. In particular embodiments, the minimal promoter includes a T7 polymerase promoter (GGGAGA). In particular embodiments, the Kozak sequence includes the sequence GCCRCC wherein R is A or G. In particular embodiments, the mini-enhancer sequence includes the sequence: CAUACUCA. In particular embodiments, the engineered 5′ UTR includes the sequence CAUACUCA, GGGAGACAUACUCAGCCACC (SEQ ID NO: 1), or GGGAGACAUACUCAGCCGCC (SEQ ID NO: 2). In particular embodiments, the engineered 5′ UTR further includes a start codon. In particular embodiments, the 5′ UTR with a start codon includes the sequence GGGAGACAUACUCAGCCACCAUG (SEQ ID NO: 3) or GGGAGACAUACUCAGCCGCCAUG (SEQ ID NO: 4).

In particular embodiments, the engineered 3′ UTR is no more than fifty nucleotides. In particular embodiments, the engineered 3′ UTR includes a spacer and a stem loop sequence. In particular embodiments, the stem loop sequence is formed by hybridizing sequences such as a) CCUC and GAGG, b) GAGG and CCUC, c) CUCC and GGAG, or d) GGAG and CUCC, wherein any set of sequences is separated by no fewer than seven nucleotides (e.g., UAACGGUCUU (SEQ ID NO: 5), also referred to as loop segment). In particular embodiments, the spacer includes the sequence: [N₁₋₃]AUA, [N₁₋₃]AAA, UGCAUA, or UGCAAA. In particular embodiments, the engineered 3′ UTR further includes a stop codon, wherein the stop codon includes the sequence UAA, UAG, or UGA. In particular embodiments, the engineered 3′ UTR includes the sequence as set forth SEQ ID NOs: 28, 29, 30, 31, 32, 33, 34, 35, 36, 37, 38, 39, 40, 41, 42, 43, 44 or 45.

In particular embodiments, the EEC is produced in vitro. In particular embodiments, the EEC does not include modified nucleotides. In particular embodiments, the EEC does not include micro-RNA binding sites. In particular embodiments, the methods disclosed herein result in iPSC that are free from any nucleic acid integration. In particular embodiments, the methods disclosed herein result in iPSC that are xeno-free.

In particular embodiments, somatic cells include mammalian cells. In particular embodiments, the mammalian cells include primate cells. In particular embodiments, the primate cells include human cells.

In particular embodiments, methods of reprogramming disclosed herein can utilize microRNA or immune evading factors (IEF) within reprogramming media. The IEF (e.g., B18R, E3, K3) can be provided in expressible and/or protein-based forms.

### BRIEF DESCRIPTION OF THE FIGURES

Some of the drawings submitted herewith may be better understood in color. Applicant considers the color versions of the drawings as part of the original submission and reserves the right to present color images of the drawings in later proceedings.

FIG. 1. Schematic of an engineered expression construct (EEC) designed to drive expression or reprogramming factors (RF). The EEC contains several modules within it to drive expression of RF. Modules located within the 5′ UTR are divided into three modules: module 1 (“M1”), which represents a promoter (e.g., a T7 promoter hexamer); module 2 (“M2”) is a unique translational enhancer (described herein); and module 3 (“M3”) is the Kozak consensus sequence. The 3′ UTR can also be divided into three segments including a stop codon, a spacer, and a stem loop structure. A polyA tail can also be included.

FIGS. 2A-2F. Octamer-binding transcription factor 4 (Oct4, also referred to as Oct3/4) expression in human foreskin fibroblasts after transfecting Oct4 mRNA containing either the engineered 5′ and/or 3′ UTRs that utilize unmodified nucleotides (UO: unmodified mRNA Oct4, UMD: unmodified mRNA MyoD-Oct4) or non-engineered sequences that use the modified nucleotide N1-methyl-pseudouridine (PUO: mRNA Oct4 using the modified nucleotide, PUMD: mRNA MyoD-Oct4 using the modified nucleotide N1-methyl-pseudouridine). Flow cytometry results show Oct4 expression upon transfection with (2A) 0 ng Oct4 mRNA, (2B) 800 ng PUO Oct4 mRNA, (2C) 800 ng PUMD Oct mRNA, (2D) 800 ng UMD Oct4 mRNA, or (2E) 800 ng UO Oct4 mRNA. (2F) Table of % Oct4 positive cells for each treatment group. Results show that when using 800 ng of mRNA per transfection, Oct4 expression was the highest using the mRNA from unmodified nucleotides (UO). The unmodified groups using 800 ng of mRNA resulted in the highest percentage of Oct4-positive cells at 50.7%. Further, the percentage of Oct4-positive cells was significantly lower using mRNAs having the modified nucleoside N1-methyl-pseudouridine (PUO and PUMD) than the mRNAs (36.9% compared to 50.7%).

FIGS. 3A-3D. Diagram of the expression of hOCT3/4 protein in HEK293 cells 24 hours after transfection with hOCT3/4 mRNA harboring the 5′-3′ UTR variants (SEQ ID NO: 84). (3A) Results from representative flow cytometry experiments indicate the intensity of hOCT3/4 and percentage of hOCT3/4 positive HEK293 cells transfected with increasing amounts of hOCT3/4 mRNA (with full-length 5′-3′ UTRs) (SEQ ID NO: 84). (3B) Results from representative flow cytometry experiments display the intensity of hOCT3/4 and percentage of hOCT3/4+ cells transfected with 1.2 pmole of hOCT3/4 with the UTRs variants (No UTR, Kozak only, 5′UTR only, 3′UTR only, Kozak and 3′ UTR, and 5′-3′ UTRs (SEQ ID NO: 84)). (3C) Line graph displays the results from the flow cytometry experiment in FIG. 3A. (3D) Bar graphs representing the results from {number of experiments=2} experiments as shown in FIG. 3B. Graph shows the level of hOCT3/4 when cells are transfected with no RNA, hOCT3/4 mRNA without UTR, hOCT3/4 mRNA and the Kozak sequence, hOCT3/4 mRNA and a 5′UTR, hOCT3/4 mRNA and a 3′UTR, hOCT3/4 mRNA with a Kozak sequence and 3′UTR, and hOCT3/4 mRNA with 5′ and 3′ UTRs (SEQ ID NO: 84). The 5′ UTR described in this figure includes SEQ ID NO: 1 and the 3′ UTR described in this figure includes SEQ ID NO: 34.

FIGS. 4A-4E. Flow cytometry results show OCT4 expression upon transfection of (4A) 0 ng Oct4 mRNA, (4B) 200 ng Oct4 mRNA, (4C) 400 ng Oct4 mRNA, or (4D) 800 ng Oct4 mRNA. (4E) A table of the percentage of Oct4 positive cells is shown for each Oct4 mRNA dose. There is a 200-fold increase in the number of human foreskin fibroblasts expressing OCT4 upon transfection of 200 ng of the engineered Oct4 mRNA (SEQ ID NO: 84).

FIGS. 5A-5E. Flow cytometry results show SOX2 expression upon transfection of (5A) 0 ng Sox2 mRNA, (5B) 200 ng Sox2 mRNA, (5C) 400 ng Sox2 mRNA, or (5D) 800 ng Sox2 mRNA. (5E) A table of the percentage of Sox2 positive cells is shown for each Sox2 mRNA dose. There is a 30-fold or 50-fold increase in the number of human foreskin fibroblasts expressing Sox2 upon transfection of 200 ng or 800 ng, respectively, of the engineered Sox2 mRNA (SEQ ID NO: 86).

FIGS. 6A-6E. Flow cytometry results show LIN28 expression upon transfection of (6A) 0 ng Lin28 mRNA, (6B) 200 ng Lin28 mRNA, (6C) 400 ng Lin28 mRNA, or (6D) 800 ng Lin28 mRNA. (6E) A table of the percentage of Lin28 positive cells is shown for each Lin28 mRNA dose. There is a 43-fold increase in the number of human foreskin fibroblasts expressing LIN28 upon transfection of 200 ng of the engineered Lin28 mRNA (SEQ ID NO: 88).

FIGS. 7A-7E. Flow cytometry results show NANOG expression upon transfection of (7A) 0 ng Nanog mRNA, (7B) 200 ng Nanog mRNA, (7C) 400 ng Nanog mRNA, or (7D) 800 ng Nanog mRNA. (7E) A table of the percentage of Nanog positive cells is shown for each Nanog mRNA dose. There is a 2-fold increase in the number of human foreskin fibroblasts expressing NANOG upon transfection of 200 ng of the engineered Nanog mRNA (SEQ ID NO: 85).

FIGS. 8A-8F. Results from a representative reprogramming experiment of neonatal human foreskin fibroblasts. (8A) Induced pluripotent stem cell (iPSC) colonies are shown forming upon reprogramming of human foreskin fibroblasts with a cocktail including engineered mRNA RF (Oct4, Sox2, Nanog, Lin28, cMyc, Klf4), non-engineered synthetic mRNA Immune Evasion Factors (IEF) (B18R, E3, K3) and miRNA. (8B) Human foreskin fibroblasts-derived iPSC isolated from single reprogrammed colonies and expanded in culture for 4 passages. (8C) Results from a flow cytometry characterization of human foreskin fibroblasts-derived iPSC for expression of differentiation marker SSEA-1 and pluripotency markers SSEA-4 and OCT3/4. (8D-8F) Flow cytometry histograms show protein expression of individual markers from FIG. 8C measured against unstained control samples (8D) SSEA-1 positive cells, (8E) SSEA4-positive cells, and (8F) Oct3/4 positive cells.

FIGS. 9A-9H. Results from a representative reprogramming experiment of adult human dermal fibroblasts. (9A) iPSC colonies are shown forming upon reprogramming the adult human dermal fibroblasts using a Reprogramming Cocktail including an EEC including mRNA RF (Oct4, Sox2, Nanog, Lin28, cMyc, Klf4), non-engineered synthetic mRNA IEF (B18R, E3, K3) and miRNAs. (9B) Human adult dermal fibroblast-derived iPSC isolated from reprogrammed colonies and expanded in culture for 5 passages. (9C) Results from the flow cytometry characterization of the human adult dermal fibroblasts-derived iPSC expanded in culture for 5 passages for expression of differentiation marker SSEA-1 and pluripotency markers SSEA-4, OCT3/4 (Oct4), Sox2, and Nanog are shown. (9D-9H) Flow cytometry histograms show protein expression of individual markers from FIG. 9C measured against unstained control samples and isotype control samples for the corresponding antibody.

FIGS. 10A-101. Results from a representative reprogramming experiment of adult human dermal fibroblasts. (10A) Bright-field microscope images of several iPSC colonies formed upon reprogramming the adult human dermal fibroblasts using a reprogramming cocktail including an EEC including mRNA RF (Oct4, Sox2, Nanog, Lin28, cMyc, Klf4), mRNA IEF (B18R, E3, K3) and miRNAs (colonies are indicated by arrows). (10B) Fluorescent microscope images of the colonies depicted in FIG. 10A are shown after live-staining with a fluorescent antibody that recognizes the TRA-1-60, a surface marker expressed by iPSC. (10C) The human adult dermal fibroblast-derived iPSC isolated from reprogrammed colonies and expanded in culture for 3 passages are shown. (10D) Results from the flow cytometry characterization of the human adult dermal fibroblasts-derived iPSC expanded in culture for 5 passages for expression of differentiation marker SSEA-1 and pluripotency markers SSEA-4, OCT3/4 (Oct4), Sox2, and Nanog are shown. (10D-101) Flow cytometry histograms show protein expression of individual markers from FIG. 10C measured against unstained control samples and isotype control samples for the corresponding antibody.

FIGS. 11A-11E. Results from a representative reprogramming experiment of adult human dermal fibroblasts (11A-11C) and human foreskin fibroblasts (11D, 11E). (11A, 11B) Bright-field microscope images are shown of several iPSC colonies formed upon reprogramming the adult human dermal fibroblasts using a reprogramming cocktail including an EEC including mRNA RF (Oct4, Sox2, Nanog, Lin28, cMyc, Klf4, SV40Tag), engineered mRNA IEF (B18R, E3, K3) and miRNAs (colonies are indicated by arrows). (11C) Fluorescent microscope images are shown of the colonies depicted in FIG. 11B after live-staining with a fluorescent antibody that recognizes the TRA-1-60, a surface marker expressed by iPSC. Human adult dermal fibroblast-derived iPSC isolated from reprogrammed colonies and expanded in culture for 3 passages. (11D, 11E) Bright-field microscope images—low and high magnification—of iPSC colonies formed upon reprogramming the human foreskin fibroblasts using a reprogramming cocktail including engineered mRNA RF (Oct4, Sox2, Nanog, Lin28, cMyc, Klf4, SV40Tag), engineered mRNA IEF (B18R, E3, K3) and miRNAs after staining for Alkaline Phosphatase activity, a biomarker of stem cells.

FIGS. 12A-12C. Results from a representative reprograming experiment of adult human dermal fibroblasts. (12A) Bright-field microscope images of several iPSC colonies formed upon reprogramming the adult human dermal fibroblasts using a reprogramming cocktail including engineered mRNA RF (Oct4, Sox2, Nanog, Lin28, cMyc, Klf4), purified B18R protein and miRNAs (colonies are indicated by arrows). (12B) Fluorescent microscope images of the colonies depicted in FIG. 12A are shown after live-staining with a fluorescent antibody that recognizes the TRA-1-60, a surface marker expressed by iPSC. (12C) Bright-field microscope images of iPSC colonies formed upon reprogramming the adult human dermal fibroblasts after staining for Alkaline Phosphatase activity, a biomarker of stem cells.

FIG. 13. Sequences supporting the disclosure include DNA sequences including: EGFP (SEQ ID NO: 69), PAC (SEQ ID NO: 70), OCT4 (SEQ ID NO: 71), NANOG (SEQ ID NO: 72), MYC (T58A) (SEQ ID NO: 73), V4 (SV40) (SEQ ID NO: 74), V5 (B18R) (SEQ ID NO: 75), V6 (VACCINIA E3) (SEQ ID NO: 76), V7 (CAMELPOX K3) (SEQ ID NO: 77), KLF4_version2 (SEQ ID NO: 78), LIN28_version2 (SEQ ID NO: 79), SOX2_version2 (SEQ ID NO: 80), and Simian virus 40 large T antigen gene, partial cds (SEQ ID NO: 81); RNA sequences including: EGFP coding sequence with 5′ and 3′ UTR (SEQ ID NO: 82), PAC coding sequence with 5′ and 3′ UTR (SEQ ID NO: 83), OCT4 coding sequence with 5′ and 3′ UTR (SEQ ID NO: 84), NANOG coding sequence with 5′ and 3′ UTR (SEQ ID NO: 85), SOX2 coding sequence with 5′ and 3′ UTR (SEQ ID NO: 86), MYC (T58A) coding sequence with 5′ and 3′ UTR (SEQ ID NO: 87), LIN28 coding sequence with 5′ and 3′ UTR (SEQ ID NO: 88), KLF4 coding sequence with 5′ and 3′ UTR (SEQ ID NO: 89), V4 (SV40) coding sequence with 5′ and 3′ UTR (SEQ ID NO: 90), V5 (B18R) coding sequence with 5′ and 3′ UTR (SEQ ID NO: 91), V6 (VACCINIA E3) coding sequence with 5′ and 3′ UTR (SEQ ID NO: 92), V7 (CAMELPOX K3) coding sequence with 5′ and 3′ UTR (SEQ ID NO: 93), KLF4_version2 coding sequence with 5′ and 3′ UTR (SEQ ID NO: 105), LIN28_ version2 coding sequence with 5′ and 3′ UTR (SEQ ID NO: 106), SOX2_version2 coding sequence with 5′ and 3′ UTR (SEQ ID NO: 107); and protein sequences including: OCT4 (SEQ ID NO: 94), NANOG (SEQ ID NO: 95), MYC (SEQ ID NO: 96), SV40 (SEQ ID NO: 97), B18R (SEQ ID NO: 98), E3 (SEQ ID NO: 99), K3 (SEQ ID NO: 100), KLF4 (SEQ ID NO: 101), LIN28 (SEQ ID NO: 102), and SOX2 (SEQ ID NO: 103).

### DETAILED DESCRIPTION

Significant scientific strides in the use of RNA as a potential source to reprogram somatic cells in vitro have been made (US Publication No. 20060247195; U.S. Pat. No. 10,772,975; WO2009077134; WO2009127230; WO2017120089; and WO2019241569). One of the largest problems to overcome is achieving high enough levels of protein expression to allow for the reprogramming. Several attempts have been made to address this problem and increase expression of at least one of Octamer-binding transcription factor 4 (Oct4), SRY-box transcription factor (Sox, e.g., Sox1, Sox2, Sox3, Sox15, and/or Sox18), Krüppel-like factor (Klf4, Klf1, and/or Klf5), Nanog, Lin28, Myc (e.g., c-Myc, N-Myc, and/or L-Myc), or SV40 large T antigen (SV40Tag). Improved methods of expressing reprogramming factors (RF) are needed.

Untranslated regions (UTRs) of a gene are transcribed but not translated. Generally, the 5′ UTR starts at the transcription start site and continues to the start codon but does not include the start codon; the 3′UTR starts following the stop codon and continues until the transcriptional termination signal. Messenger RNAs (mRNAs) include UTRs that are shown to recruit ribosomes, initiate translation and thereby drive protein expression. While according to the preceding description start and stop codons are not generally considered part of UTRs, in the current disclosure, these segments are sometimes included within sequences designated as UTRs to create operational segments.

There is a growing body of evidence about the regulatory roles played by UTRs in terms of stability of nucleic acid molecules and resulting translation/protein expression. Sequences within UTRs differ in prokaryotes and eukaryotes. For example, the Shine-Dalgarno consensus sequence (5′-AGGAGGU-3′) recruits ribosomes in bacteria while the RNA Kozak consensus sequence (5′-GCCRCCRUGG-3′ (SEQ ID NO: 6)) boosts translation initiation events in mammalian cells and can further include the initiation codon (AUG).

The ‘R’ in the Kozak consensus sequence represents either A or G. The −3 position of the Kozak consensus sequence enhances translation initiation, and as a whole, the Kozak sequence is believed to stall the translation initiation complex for the proper recognition of the start codon. While the Kozak consensus sequence, by itself, can drive ribosomal scanning and translational initiation, additional UTRs associated with highly abundant proteins in the human transcriptome were analyzed. Studies suggest the relative abundance of proteins associated with genetic information processing, including chromosomal and ribosomal associated proteins (Beck et al., Mol. Syst. Biol. (2011), doi: 10.1038/msb.2011.82; Liebermeister et al., Proc. Natl. Acad. Sci. U. S. A. (2014), doi: 10.1073/pnas. 1314810111). For example, the alignment of the 5′ UTRs of highly-expressed ribosomal-associated proteins (RPLs/RPSs) illustrate the appearance of the 5′ Terminal OligoPyrimidine Track (i.e. 5′TOP) or C/U (Lavallee-Adam et al., Nucleic Acids Res. (2017), doi: 10.1093/nar/gkx751; Yoshihama et al., Genome Res. (2002), doi: 10.1101/gr.214202; Cardinali et al., J. Biol. Chem. (2003), doi: 10.1074/jbc.M300722200; Pichon et al., Curr. Protein Pept. Sci. (2012), doi: 10.2174/13892031280161947). Generally, 5′TOP sequences are located near the start codon and are important in transcription (i.e. RNA synthesis) and translation of transcripts.

According to the present disclosure, several engineered sequences for the 5′ UTR and 3′ UTR are provided for use in expressing at least one of the RF mRNAs to create induced pluripotent stem cells (iPSC). Furthermore, these engineered sequences for the 5′ UTR and 3′ UTR are shown to be useful in increasing the protein expression of at least one of the RF when they are used flanking the given open reading frame. These RF include, Oct4, Sox, Klf, Nanog, Lin28, Myc, or SV40Tag. Moreover, these engineered 5′ UTR and 3′ UTR sequences with at least one of the RF are shown to work similarly in different cell types, including fibroblasts, hematopoietic stem cells, mesenchymal stem cells, and murine fibroblast, irrespective of the manner of transfection. According to certain embodiments, the engineered stem loop as described herein, is not dependent upon the sequence, merely that it can form a stem loop with the sequence, indicating that the secondary structure may be important.

By engineering the features typically found in abundantly expressed genes of specific target organs, one can increase protein expression of the coding sequences. For example, introduction of 5′ UTR of liver-expressed mRNA, such as albumin, serum amyloid A, Apolipoprotein A/B/E, transferrin, alpha fetoprotein, erythropoietin, or Factor VIII, can be used to enhance expression of a coding sequences in hepatic cell lines or liver. Likewise, use of 5′ UTR from other tissue-specific mRNA to improve expression in that tissue is possible for muscle (MyoD, Myosin, Myoglobin, Myogenin, Herculin), for endothelial cells (Tie-1, CD36), for myeloid cells (C/EBP, AML1, G-CSF, GM-CSF, CD11b, MSR, Fr-1, i-NOS), for leukocytes (CD45, CD18), for adipose tissue (CD36, GLUT4, ACRP30, adiponectin) and for lung epithelial cells (SP-A/B/C/D).

UTRs, however, can be 100s to 1000s of nucleotides (nts) in length. In certain examples, engineered expression constructs (EEC) disclosed herein were designed to have minimal UTRs (minUTs). That is the EEC were designed to have 5′ and/or 3′ UTR that are as minimal as possible and still allow for high levels of RF.

Thus, according to certain aspects, the current disclosure provides minimal UTRs to drive expression of one or more RF. In certain examples, the current disclosure provides 5′ UTR with 20-23 nucleotides. In certain examples, the current disclosure provides 3′ UTR with 27 nucleotides or 67 nucleotides, depending on whether an optional polyA tail is included. Together then, certain examples of 5′ and 3′ UTR combinations include 47-50 nucleotides or 87-90 nucleotides.

According to certain aspects, to construct minUTs, the current disclosure integrates elements necessary for the production of mRNAs in vitro and protein in vivo. For the production of mRNAs in vitro, the current disclosure can employ the use of RNA polymerase from the T7 bacteriophage (T7P). T7P binds to a specific DNA double-helix sequence (5′-TAATACGACTCACTATAG-3′ (SEQ ID NO: 7)) and initiates RNA synthesis with the incorporation of guanosine (the last G in the promoter; underlined) as the first ribonucleotide. This binding sequence is generally followed by a pentamer (5′-GGAGA-3′) that serves to stabilize the transcriptional complex, promote T7P clearance and extension of the RNA polymer.

In particular embodiments, 5′ UTRs include a promoter (GGAGA), a mini-enhancer sequence (CAUACUCA, herein), and a Kozak sequence, such as a truncated form of the Kozak sequence (GCCRCC). In particular embodiments, a 5′ UTR is described as also being operably linked to a start codon to create an operational segment.

In particular embodiments, minimal promoters are selected for use within a 5′ UTR. Minimal promoters have no activity to drive expression on their own but can be activated to drive expression when linked to a proximal enhancer element. In particular embodiments, the minimal promoter includes a segment of a minimal T7 promoter (mini-T7 promoter).

Certain examples of disclosed 5′ UTR include a unique mini-enhancer sequence (CAUACUCA). The mini-enhancer sequence can be located between a minimal promoter (e.g., GGAGA) and the Kozak consensus sequence to generate a minimal 5′ UTR with 20-23 nucleotides (depending on whether a start codon is designated as part of the UTR). Eukaryotic translation generally starts with the AUG codon, however other start codons can be included. Mammalian cells can also start translation with the amino acid leucine with the help of a leucyl-tRNA decoding the CUG codon and mitochondrial genomes use AUA and AUU in humans. These components and exemplary 5′ UTR are provided in Table 1.

TABLE 1
5′ UTR Components and Constructs.

| SEQ ID NO: | Sequence | Description |
|---|---|---|
| N/A | CAUACUCA | Mini-enhancer sequence |
| N/A | GGGAGA | Mini-T7 promoter |
| N/A | GCCRCCAUG | Kozak Consensus Sequence |
| 1 | GGGAGACAUACUCAGCCACC | 5′ UTR Construct with “A” Kozak |
| | | Sequence (no start codon) |
| 2 | GGGAGACAUACUCAGCCGCC | 5′ UTR Construct with “G” Kozak |
| | | Sequence (no start codon) |
| 3 | GGGAGACAUACUCAGCCACCAUG | 5′ UTR Construct with “A” Kozak |
| | | Sequence (with start codon) |
| 4 | GGGAGACAUACUCAGCCGCCAUG | 5′ UTR Construct with “G” Kozak |
| | | Sequence (with start codon) |

In certain examples, 5′ UTR are capped. For example, eukaryotic mRNAs are guanylylated by the addition of inverted 7-methylguanosine to the 5′ triphosphate (i.e. m7GpppN where N denotes the first base of the mRNA). The m7GpppN or the 5′ cap structure of an mRNA is involved in nuclear export and binds the mRNA Cap Binding Protein (CBP), which is responsible for mRNA translation competency.

The ribose sugars of the first and second nucleotides of mRNAs may optionally also be methylated (i.e., addition of CH3 group) at the 2′-Oxygen (i.e., 2′O) position. A non-methylated mRNA at first and second nucleotides is denoted as Cap0 (i.e., m7GpppN), whereas methylation at the 2′ O on the first and second nucleotides are denoted as Cap1 (i.e. m7GpppNm) and Cap2 (i.e. m7GpppNmNm), respectively. Furthermore, if the first 5′ nucleotide is adenosine, it may further be methylated at the 6th Nitrogen (6N) position (i.e. m7Gpppm6A) or form modified Cap0 (i.e. m7Gpppm6A) or modified Cap1 (i.e., m7Gpppm6Am) or modified Cap2 (i.e. m7Gpppm6AmNm).

The RNA guanylylation or the addition of Cap0 (i.e. m7GpppN) may be achieved enzymatically in vitro (i.e. after the RNA synthesis) by Vaccinia Virus Capping Enzyme (VCE). The creation of Cap1 and Cap2 structures may further be achieved enzymatically via the addition of mRNA 2′-O-methyltransferase and S-adenosyl methionine (i.e. SAM). Alternatively, the Cap structure may be added co-transcriptionally in vitro by the incorporation of Anti-Reverse Cap Analog (i.e. ARCA). ARCA is methylated at the 3′-oxygen (3′O) on the cap (m73′OmGpppN) to ensure the incorporation of the cap structure in the correct orientation. In particular embodiments, any of the above cap structures may be used for an EEC.

In particular embodiments, the 5′ UTR is operably linked to a RF coding sequence. As used herein, the term “operably linked” refers to a functional linkage between a nucleotide expression control sequence (e.g., a promoter sequence or a UTR) and another nucleotide sequence, whereby the control sequence allows for and results in the transcription and/or translation of the other nucleotide sequence.

The current disclosure also provides 3′ UTR for optional use with disclosed 5′ UTR.

3′ UTRs are known to have stretches of adenosines and uridines embedded in them. These AU rich signatures are particularly prevalent in genes with high rates of turnover. Based on their sequence features and functional properties, the AU rich elements (AREs) can be separated into three classes: Class I AREs contain several dispersed copies of an AUUUA motif within U-rich regions. Myc and MyoD contain class I AREs. Class II AREs possess two or more overlapping UUAUUUA (U/A) (U/A) nonamers.

As indicated previously, disclosed 3′ UTR include a spacer, a stem loop structure, and optionally, a polyadenine tail (polyA tail). In certain examples herein, the 3′ UTR is also depicted as being operably linked to a stop codon.

Exemplary stop codons include UAA, UGA, and UAG.

Exemplary spacers include [N₁₋₃]AUA and [N₁₋₃]AAA (e.g., UGCAUA, UGCAAA, UGAAA, GCAUA, UAAA, and GAUA), wherein N is any nucleotide including A, G, C, T, or U. The subscript numbers indicate the quantity of the nucleotide. For example [N₁₋₃] includes 1, 2, or 3 nucleotides as set forth in N, NN, or NNN.

Stem loops (SL), or hairpin or hairpin loops, are a feature of highly expressed transcripts within the 3′ UTR. The SLs are distinct secondary structures where complementary nucleotides are paired as the double helix (or the stem) often interrupted with sequences that form the loop. The particular secondary structure represented by the SL includes a consecutive nucleic acid sequence including a stem and a (terminal) loop, also called hairpin loop, wherein the stem is formed by two neighbored entirely or partially reverse complementary sequence elements (hybridizing sequence); which are separated by a short sequence (e.g. 7-15 nucleotides), which forms the loop of the SL structure. The two neighbored entirely or partially complementary sequences may be defined as e.g., SL elements stem 1 and stem 2. The SL is formed when these two neighbored entirely or partially reverse complementary sequences, e.g., SL elements stem 1 and stem 2, form base-pairs with each other, leading to a double stranded nucleic acid sequence including an unpaired loop at its terminal ending formed by the short sequence located between SL elements stem 1 and stem 2. Thus, an SL includes two stems (stem 1 and stem 2), which—at the level of secondary structure of the nucleic acid molecule-form base pairs with each other, and which—at the level of the primary structure of the nucleic acid molecule—are separated by a short sequence that is not part of stem 1 or stem 2. For illustration, a two-dimensional representation of the SL resembles a lollipop-shaped structure. The formation of a stem loop structure requires the presence of a sequence that can fold back on itself to form a paired double strand; the paired double strand is formed by stem 1 and stem 2. The stability of paired SL elements is typically determined by the length, the number of nucleotides of stem 1 that are capable of forming base pairs (preferably canonical base pairs, more preferably Watson-Crick base pairs) with nucleotides of stem 2, versus the number of nucleotides of stem 1 that are not capable of forming such base pairs with nucleotides of stem 2 (mismatches or bulges). According to the present disclosure, the optimal loop length is 7-15 nucleotides, such as 7 nucleotides, 8 nucleotides, 9 nucleotides, 10 nucleotides, 11 nucleotides, 12, nucleotides, 13 nucleotides, 14 nucleotides, or 15 nucleotides. If a given nucleic acid sequence is characterized by an SL, the respective complementary nucleic acid sequence is typically also characterized by an SL. An SL is typically formed by single-stranded RNA molecules. In particular embodiments, the SL length is at least 7 nucleotides, at least 8 nucleotides, at least 9 nucleotides, at least 10 nucleotides, at least 11 nucleotides, at least 12 nucleotides, at least 13 nucleotides, at least 14 nucleotides, at least 15 nucleotides, at least 16 nucleotides, at least 17 nucleotides, at least 18 nucleotides, at least 19 nucleotides, at least 20 nucleotides, at least 21 nucleotides, at least 22 nucleotides, or at least 23 nucleotides in length.

The SLs are present within 3′ UTR of highly expressed transcripts (e.g. those coding for abundant cellular proteins like histones) where it boosts translation, regardless of polyadenine tail (Gallie et al., Nucleic Acids Res. (1996), doi: 10.1093/nar/24.10.1954). The histone 3′ UTR stem consensus is characterized as six base-pairs, two of which are G-C pairs, three pyrimidine-purine (Y-R) pairs and one A-U pairs and moreover, the loop includes 4 ribonucleotides with two uridines (U), one purine (Y) and one ribonucleotide (N) (Gallie et al., The histone 3′-terminal stem loop structure is necessary for translation in Chinese hamster ovary cells. (Nucleic Acids Res. (1996), doi: 10.1093/nar/24.10.1954; Tan et al., Science (80). (2013), doi: 10.1126/science. 1228705; Battle & Doudna, RNA (2001), doi: 10.1017/S1355838201001820).

The SLs associate with stem loop binding proteins (SLBPs) for replication-dependent mRNA stability/processing/metabolism/translation. Structural evidence suggests that the direct contact of SLBPs with SLs occurs at a guanosine nucleotide at the base of SL (G7) (Tan et al., Science (80). (2013), doi: 10.1126/science.1228705; Battle & Doudna, RNA (2001), doi: 10.1017/S1355838201001820). Furthermore, the adjacent adenosines, or more specifically, upstream AAA, to the stem impact SLBP binding and function (Battle & Doudna, RNA (2001), doi: 10.1017/S1355838201001820; William & Marzluff, Nucleic Acids Res. (1995), doi: 10.1093/nar/23.4.654). The 3′ UTR also serves to stabilize protein-coding transcripts while increasing their translational capacity. According to certain embodiments, the current disclosure provides designs for synthetic SLs to incorporate the features of SL: three groups of G-C pairs interrupted by a sequence (UAACGGUCUU (SEQ ID NO: 5)) with adjacent spacer sequences such as adenosines to increase SLBP binding and mRNA translation.

Importantly, the stem loops used in EEC are not sequence-orientation dependent and may include a) CCUC and GAGG, b) GAGG and CCUC, c) CUCC and GGAG, or d) GGAG and CUCC. Further, the distance between the two arms of the stem (where the CCUC and GAGG base pair) needs to be long enough for a loop to form. In particular embodiments, stem loops can include complementary sequences such as a) RRRR and YYYY, b) RYRR and YRYY, c) RRYR and YYRY, d) RRRY and YYYR, e) RYYR and YRRY, f) RRYY and YYRR, g) YYRR and RRYY, h) YYYR and RRRY, or i) RYYY and YRRR, wherein R is purine (A or G) and Y is a pyrimidine (e.g., U or C).

According to certain embodiments, the number of nucleotides between the two arms may be seven, eight, nine, ten, or longer nucleotides. Preferred embodiments of the length between the two arms of the stem loop are no shorter than seven nucleotides (loop segment). In certain examples, the loop segment of an SL includes UAACGGUCUU (SEQ ID NO: 5). In particular embodiments, an SL sequence includes CCUCUAACGGUCUUGAGG (SEQ ID NO: 54), GAGGUAACGGUCUUCCUC (SEQ ID NO: 55), CUCCUAACGGUCUUGGAG (SEQ ID NO: 56), GGAGUAACGGUCUUCUCC (SEQ ID NO: 57), CCUCUAACUGUGAGG (SEQ ID NO: 58), GAGGUAACGCUCUCCUC (SEQ ID NO: 59), CUCCUAACGGUCGUGGGAG (SEQ ID NO: 60), GGAGGGUAACCGUCUUCUCC (SEQ ID NO: 61), CCUCUAACGGUCUUAGCGAGG (SEQ ID NO: 62), or GAGGUAACGUAACGGUCUUCCUC (SEQ ID NO: 63). In other embodiments, an SL sequence includes GAUGCCCCAUUCACGAGUAGUGGGUAUU (SEQ ID NO: 8), GGCACCCUGCGCAGGUGAUGCAGGUGCC (SEQ ID NO: 9), GUUCGCUCGGUCAGGAGAGCUGACGGAC (SEQ ID NO: 10), UCUUACAGUGGCAUGUGACCGUUUAAGG (SEQ ID NO: 11), CGCGGCGCAUGCACGUGACAUGCCUGCG (SEQ ID NO: 12), CGGUCCCGUGGCAAGAGUCUAUGGAUUG (SEQ ID NO: 13), AUGUUCGGCUCCAAGAGCGAGUUGAUAU (SEQ ID NO: 14), CGAUUCGGGCACAUGUGCUGUCUGAUUG (SEQ ID NO: 15), GUAUUCUGAUGCACGUGCCAUCAAGUAC (SEQ ID NO: 16), or UUGAGCAGGAUCAAGUGCAUUCUUUCAA (SEQ ID NO: 17). In particular embodiments, an SL sequence includes RRYRYYYYRYYYRYRRRYRRYRRRYRYY (SEQ ID NO: 18), RRYRYYYYRYRYRRRYRRYRYRRRYRYY (SEQ ID NO: 19), RYYYRYYYRRYYRRRRRRRYYRRYRRRY (SEQ ID NO: 20), YYYYRYRRYRRYRYRYRRYYRYYYRRRR (SEQ ID NO: 21), YRYRRYRYRYRYRYRYRRYRYRYYYRYR (SEQ ID NO: 22), YRRYYYYRYRRYRRRRRYYYRYRRRYYR (SEQ ID NO: 23), RYRYYYRRYYYYRRRRRYRRRYYRRYRY (SEQ ID NO: 24), YRRYYYRRRYRYRYRYRYYRYYYRRYYR (SEQ ID NO: 25), RYRYYYYRRYRYRYRYRYYRYYRRRYRY (SEQ ID NO: 26), or YYRRRYRRRRYYRRRYRYRYYYYYYYRR (SEQ ID NO: 27), wherein R is purine (A or G) and Y is a pyrimidine (e.g. U or C). See Gorodkin et al., (Nucleic Acids Research 29 (10): 2135-2144, 2001) for additional exemplary SL motifs.

Optional Poly-A Tails. During natural RNA processing, a long chain of adenine nucleotides (poly-A tail) may be added to a polynucleotide such as an mRNA molecule in order to increase stability. Immediately after transcription, the 3′ end of the transcript may be cleaved to free a 3′ hydroxyl. Then poly-A polymerase adds a chain of adenine nucleotides to the RNA. This process, called polyadenylation, adds a poly-A tail that can be between, for example, 100 and 250 residues long. In in vitro RNA synthesis, a polyA tail may be in encoded on the DNA template and as such is incorporated during the in vitro transcription process.

In certain examples, a polyA tail ranges from 0 to 500 nucleotides in length (e.g., 0, 10, 20, 30, 40, 50, 60, 70, 80, 90, 120, 140, 160, 180, 200, 250, 300, 350, 400, 450, or 500 nucleotides). Certain examples utilize a 40 nucleotide polyA tail. Length may also be determined in units of or as a function of polyA Binding Protein binding. In these embodiments, the polyA tail is long enough to bind 4 monomers of PolyA Binding Protein, 3 monomers of PolyA Binding Protein, 2 monomers of PolyA Binding Protein, or 1 monomer of PolyA Binding Protein. PolyA Binding Protein monomers bind to stretches of 38 nucleotides.

Based on the foregoing discussion of the 3′ UTR, 3′ UTR constructs disclosed herein include one or more of spacers (e.g., [N₁₋₃]AUA, [N₁₋₃]AAA, UGCAUA or UGCAAA), stem loop hybridizing sequences (e.g., CCUC, GAGG, CUCC, GGAG); stem loop segments (e.g., [N7-15], UAACGGUCUU (SEQ ID NO: 5)), and/or optionally, polyA tails. When stop codons are designated as part of a 3′ UTR, exemplary stop codons include UAA, UGA, and UAG. Exemplary 3′ UTR constructs based on these components are presented in Table 2.

TABLE 2
3′ UTR Constructs.

| SEQ ID | |
|---|---|
| NO: | Sequence |
| 28 | [N₁₋₃]AUACCUC[N₇₋₁₅]GAGG |
| 29 | [N₁₋₃]AAACCUC[N₇₋₁₅]GAGG |
| 30 | [N₁₋₃]AUAGGAG[N₇₋₁₅]CUCC |
| 31 | [N₁₋₃]AAAGGAG[N₇₋₁₅]CUCC |
| 32 | [N₁₋₃]AUAGAGG[N₇₋₁₅]CCUC |
| 33 | [N₁₋₃]AAAGAGG[N₇₋₁₅]CCUC |
| 34 | Stop-UGCAUACCUCUAACGGUCUUGAGG-PolyA |
| 35 | Stop-UGCAUAGGAGUAACGGUCUUCUCC-PolyA |
| 36 | Stop-UGCAAAGGAGUAACGGUCUUCUCC-PolyA |
| 37 | UAAUGCAUACCUCUAACGGUCUUGAGG |
| 38 | UGAUGCAUACCUCUAACGGUCUUGAGG |
| 39 | UAGUGCAUACCUCUAACGGUCUUGAGG |
| 40 | UAAUGCAUAGAGGUAACGGUCUUCCUC |
| 41 | UGAUGCAUAGAGGUAACGGUCUUCCUC |
| 42 | UAGUGCAUAGAGGUAACGGUCUUCCUC |
| 43 | UAAUGCAAAGAGGUAACGGUCUUCCUC |
| 44 | UGAUGCAAAGAGGUAACGGUCUUCCUC |
| 45 | UAGUGCAAAGAGGUAACGGUCUUCCUC |

In particular embodiments, other non-UTR sequences may be incorporated into the 5′ and/or 3′ UTRs. For example, introns or portions of intron sequences may be incorporated into these regions. Incorporation of intronic sequences may increase RF expression as well as mRNA levels.

Aspects of the current disclosure are now described with more supporting options and detail as follows: (i) EEC Architectures Utilizing Disclosed 5′ and 3′ UTR; (ii) Reprogramming Factors; (iii) Stabilizing EEC; (iv) In Vitro Synthesis of EEC; (v) Cell Types; (vi) Somatic Cell Isolation; (vii) Reprogramming of Somatic Cells; (viii) iPSC Enrichment; (ix) Culture and Storage of Cells; (x) Formulations and Three-Dimensional Constructs; (xi) Differentiation of iPSC; (xii) Methods of Use; (xiii) Exemplary Embodiments; (xiv) Experimental Examples; and (xv) Closing Paragraphs. These headings are provided for organizational purposes only and do not limit the scope or interpretation of the disclosure.

### (i) EEC ARCHITECTURES UTILIZING DISCLOSED 5′ AND 3′ UTR

EEC Include an Engineered 5′ UTR Disclosed Herein and/or an Engineered 3′ UTR Disclosed Herein and a Coding Sequence within an Open Reading Frame. The Coding Sequence Encodes a Protein Useful in Reprogramming a Somatic Cell to an iPSC. The Encoded Protein can be a RF or an IEF

EEC are formed of nucleic acids. The term “nucleic acid” or “recombinant nucleic acid,” in their broadest sense, include any compound and/or substance that include a polymer of nucleotides. These polymers are often referred to as polynucleotides. Exemplary nucleic acids or polynucleotides of the present disclosure include: ribonucleic acids (RNAs), deoxyribonucleic acids (DNAs), threose nucleic acids (TNAs), glycol nucleic acids (GNAs), peptide nucleic acids (PNAs), locked nucleic acids (LNAs, including LNA having a β-D-ribo configuration, α-LNA having an α-L-ribo configuration (a diastereomer of LNA), 2′-amino-LNA having a 2′-amino functionalization, and 2′-amino-α-LNA having a 2′-amino functionalization) or hybrids thereof. In particular embodiments, polynucleotides are provided in the form of RNA, DNA expression vectors, or plasmids that encode one or more polypeptides.

The term “expression” with respect to a gene or polynucleotide refers to transcription of the gene or polynucleotide and, as appropriate, translation of an mRNA transcript to a protein or polypeptide. Thus, as will be clear from the context, expression of a protein or polypeptide results from transcription and/or translation of the open reading frame.

Traditionally, the basic components of an mRNA molecule include at least a coding region, a 5′ UTR, a 3′ UTR, a 5′ cap, and a poly-A tail. Building on this wild-type modular structure, the present disclosure expands the scope of functionality of traditional mRNA molecules by providing polynucleotides or primary RNA constructs which maintain a modular organization, but which include one or more structural alterations which impart useful properties to the polynucleotide including, in some embodiments, the lack of a substantial induction of the innate immune response of a cell into which the polynucleotide is introduced. As used herein, a “structural” feature or modification is one in which two or more linked nucleotides are inserted, deleted, duplicated, inverted or randomized in an EEC without significant chemical modification to the nucleotides themselves. Structural modifications will result in a different sequence of nucleotides. For example, the polynucleotide “ATCG” may be structurally modified to “ATCCCG”. Here, the dinucleotide “CC” has been inserted, resulting in a structural modification to the polynucleotide.

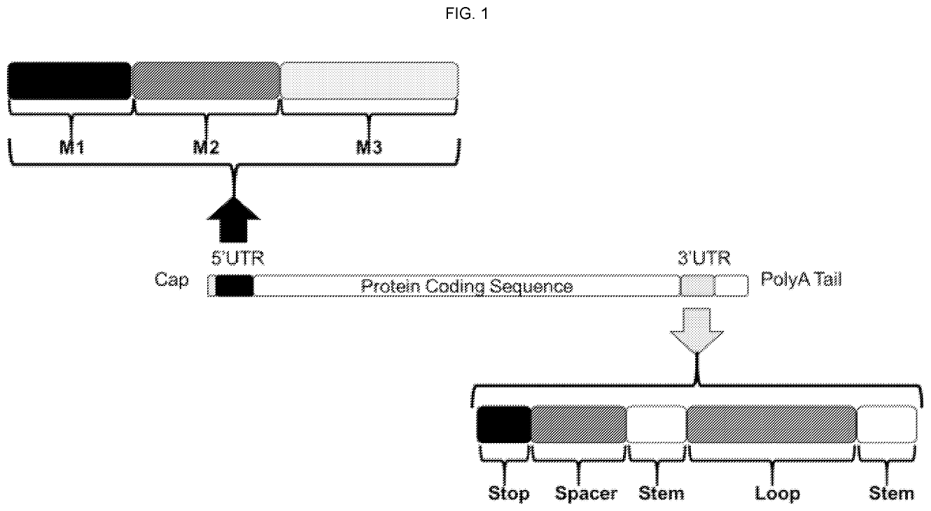

FIG. 1 shows a representative EEC of the present disclosure. In certain examples, “EEC” refers to a polynucleotide transcript having a 5′ and/or 3′ UTR disclosed herein flanking a RF coding sequence which encodes one or more RF and which retains sufficient structural and/or chemical features to allow the one or more RF encoded therein to be translated.

Returning to FIG. 1, the depicted EEC includes a RF coding sequence of linked nucleotides within an open reading frame that is flanked by a first flanking region and a second flanking region. This coding sequence includes an RNA sequence encoding one or more RF. The RF may include at its 5′ terminus one or more signal sequences encoded by a signal sequence region. The first flanking region may include a region of linked nucleotides including one or more complete or incomplete 5′ UTR sequences. The first flanking region may also include a 5′ terminal cap. Bridging the 5′ terminus of the coding sequence and the first flanking region is a first operational segment. Traditionally this operational segment includes a start codon. The operational segment may alternatively include any translation initiation sequence or signal including a start codon.

The first flanking region may include modules that are located within the 5′ UTR. This first flanking region may be divided into three modules: module 1 (“M1”), which represents a minimal promoter (e.g., T7 promoter hexamer); module 2 (“M2”) which is a unique translational enhancer (CAUACUCA, described herein); and module 3 (“M3”) which is the Kozak consensus sequence. The T7 promoter hexamer is part of the T7 polymerase promoter, which is in turn part of the T7 class III promoters, a particular class of promoters well known in the art associated with and responsible for inducing the transcription of certain promoters of the T7 bacteriophage. More specifically the T7 promoter and hexamer has the full sequence (5′-TAATACGACTCACTATAGGGAGA-3′ (SEQ ID NO: 46) and initiates RNA synthesis with the incorporation of guanosine as the first ribonucleotide leading to the promoters described herein. The Kozak consensus refers to the Kozak consensus sequence (5′-GCCRCCATGG-3′ (SEQ ID NO: 47)) where ‘R’ represents either A or G.

The second flanking region may include a region of linked nucleotides including one or more complete or incomplete 3′ UTRs. The flanking region may also include a 3′ tailing sequence (e.g., polyA tail). The 3′ UTR may be divided into three segments including the stop codon, spacer, and a stem loop structure.

Bridging the 3′ terminus of the coding sequence and the second flanking region is a second operational segment. Traditionally this operational segment includes a stop codon. The operational segment may alternatively include any translation termination sequence or signal including a stop codon. According to the present disclosure, multiple serial stop codons may also be used.

EEC may additionally include one or more elements (e.g., IRES sequences, core or mini-promoters and the like) to direct the expression of another RNA sequence. The EEC of the disclosure can include, in one embodiment, 5′ and 3′ alphavirus replication recognition sequences, coding sequences for alphavirus nonstructural proteins, a polyadenylation tract and one or more of a coding sequence encoding a RF selected from the group including Sox, Oct4, Klf, Lin28 and Nanog, and optionally Myc or SV40Tag.

Generally, the shortest length of the coding sequence of the EEC can be the length of a nucleic acid sequence that is sufficient to encode a RF.

In some embodiments, the EEC includes from 30 to 100,000 nucleotides (e.g., from 30 to 50, from 30 to 100, from 30 to 250, from 30 to 500, from 30 to 1,000, from 30 to 1,500, from 30 to 3,000, from 30 to 5,000, from 30 to 7,000, from 30 to 10,000, from 30 to 25,000, from 30 to 50,000, from 30 to 70,000, from 100 to 250, from 100 to 500, from 100 to 1,000, from 100 to 1,500, from 100 to 3,000, from 100 to 5,000, from 100 to 7,000, from 100 to 10,000, from 100 to 25,000, from 100 to 50,000, from 100 to 70,000, from 100 to 100,000, from 500 to 1,000, from 500 to 1,500, from 500 to 2,000, from 500 to 3,000, from 500 to 5,000, from 500 to 7,000, from 500 to 10,000, from 500 to 25,000, from 500 to 50,000, from 500 to 70,000, from 500 to 100,000, from 1,000 to 1,500, from 1,000 to 2,000, from 1,000 to 3,000, from 1,000 to 5,000, from 1,000 to 7,000, from 1,000 to 10,000, from 1,000 to 25,000, from 1,000 to 50,000, from 1,000 to 70,000, from 1,000 to 100,000, from 1,500 to 3,000, from 1,500 to 5,000, from 1,500 to 7,000, from 1,500 to 10,000, from 1,500 to 25,000, from 1,500 to 50,000, from 1,500 to 70,000, from 1,500 to 100,000, from 2,000 to 3,000, from 2,000 to 5,000, from 2,000 to 7,000, from 2,000 to 10,000, from 2,000 to 25,000, from 2,000 to 50,000, from 2,000 to 70,000, and from 2,000 to 100,000).

According to the present disclosure, the first and second flanking regions may range independently from 5-100 nucleotides in length (e.g., 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100 nucleotides).

According to the present disclosure, the capping region may include a single cap or a series of nucleotides forming the cap. In this embodiment the capping region may be from 1 to 10, e.g., 2-9, 3-8, 4-7, 1-5, 5-10, or at least 2, or 10 or fewer nucleotides in length. In some embodiments, the cap is absent.

According to the present disclosure, the first and second operational segments may range from 3 to 40, e.g., 5-30, 10-20, 15, or at least 4, or 30 or fewer nucleotides in length and may include, in addition to a start and/or stop codon, one or more signal and/or restriction sequences.

In one embodiment, an EEC includes a sequence that is 90%, 95%, 98%, 99% or 100% identical to the UTRs as described herein (including wherein “T” of the sequence can be substituted with “U”), and one or more RF selected from the group including Oct4, Sox, Klf, Lin28, Nanog, and optionally Myc or SV40Tag. The order of the RF is not critical to the disclosure; thus the order may be Klf, Oct4, Sox, Lin28 or can be Sox, Klf, Oct4, Lin28, or Oct4, Klf, Sox, Lin28 or any variation of the order of the RF. Where more than one RF is present, the coding sequences may be separated by an internal ribosome entry site (IRES) or a small (e.g., a core) promoter such as SP1. The EEC may further include a selectable marker (e.g., an antibiotic resistance marker). In other embodiments, coding sequences of RF (RFs) may be separated by self-cleaving peptides such as T2A and/or E2A. In another embodiment, the EEC includes from 5′ to 3′: the engineered 5′ UTR-(RF1)-(self cleaving peptide)-(RF2)-(self cleaving peptide)-(RF3)-(IRES or core promoter)-(RF4)-(IRES or optional promoter)-(optional selectable marker)-3′UTR and polyA tail; wherein RF1-4 are factors that induce de-differentiation of a somatic cell to a pluripotent cell, wherein RF2-3 are optional, RF3-4 are optional, or RF4 is optional; wherein RF1-4 are selected from the group including Oct4, Klf, Sox, Lin28, Nanog, and optionally Myc or SV40Tag.

In particular embodiments, polynucleotides can be formed or amplified using cDNA, mRNA or alternatively, genomic DNA, as a template and appropriate oligonucleotide primers according to standard polymerase chain reaction (PCR) amplification techniques and those procedures described in the Experimental Examples section below. The nucleic acid so amplified can be cloned into an appropriate vector and characterized by sequence analysis. Furthermore, oligonucleotides corresponding to nucleotide sequences can be prepared by standard synthetic techniques, e.g., using an automated DNA synthesizer.

In certain examples, EEC exclude microRNA binding sites and/or modified nucleotide triphosphates (NTPs) in the 5′ UTR, in the 3′ UTR, in the 5′ UTR and the 3′ UTR, or in the entirety of the EEC as described elsewhere herein. miRNA sequences are shown in Table 3.

TABLE 3
miRNA Sequences.

| | | SEQ | | miRBase |
|---|---|---|---|---|
| ID | Mature sequence | ID NO: | miRBase ID | Accession # |
| 302a | UAAGUGCUUCCAUGUU | 64 | hsa-miR-302a- | MIMAT0000684 |
| | UUGGUGA | | 3p | |
| 302b | UAAGUGCUUCCAUGUU | 65 | hsa-miR-302b- | MIMAT0000715 |
| | UUAGUAG | | 3p | |
| 302c | UAAGUGCUUCCAUGUU | 66 | hsa-miR-302c- | MIMAT0000717 |
| | UCAGUGG | | 3p | |
| 302d | UAAGUGCUUCCAUGUU | 67 | hsa-miR-302d- | MIMAT0000718 |
| | UGAGUGU | | 3p | |
| 367 | AAUUGCACUUUAGCAA | 68 | hsa-miR-367-3p | MIMAT0000719 |
| | UGGUGA | | | |

In certain examples, EEC include messenger RNA (mRNA). As used herein, “messenger RNA” (mRNA) refers to any polynucleotide which encodes a protein and which is capable of being translated to produce the encoded RF in vitro, in vivo, in situ, or ex vivo.

In certain examples, an EEC encoding an RF can additionally encode an IEF. In alternative embodiments, an EEC that encodes an RF does not encode and IEF, and conversely, an EEC that encodes an IEF does not encode an RF.

### (ii) REPROGRAMMING FACTORS (RF)

Herein, a “reprogramming factor” refers to a protein, for example a transcription factor, that plays a role in changing somatic cells into induced pluripotent stem cells (iPSC). The term “reprogramming factor” further includes any analogue molecule or variant that mimics the function of the factor. EEC of the present disclosure can encode RF, such as proteins or fragments thereof that are useful in producing iPSC. A “protein” refers to a polymer of amino acid residues (natural or unnatural) linked together most often by peptide bonds. The term includes polypeptides and peptides of any size, structure, or function. In some instances, the protein encoded is smaller than 50 amino acids and the protein is then termed a peptide. If the protein is a peptide, it will include at least 2 linked amino acids. Proteins include naturally occurring proteins, synthetic proteins, homologs, orthologs, paralogs, fragments, recombinant proteins, fusion proteins and other equivalents, variants, and analogs thereof. A protein may be a single protein or may be a multi-molecular complex such as a dimer, trimer, or tetramer. They may also include single chain or multichain proteins such as antibodies or insulin and may be associated or linked. Most commonly disulfide linkages are found in multichain proteins. The term protein may also apply to amino acid polymers in which one or more amino acid residues are an artificial chemical analogue of a corresponding naturally occurring amino acid.

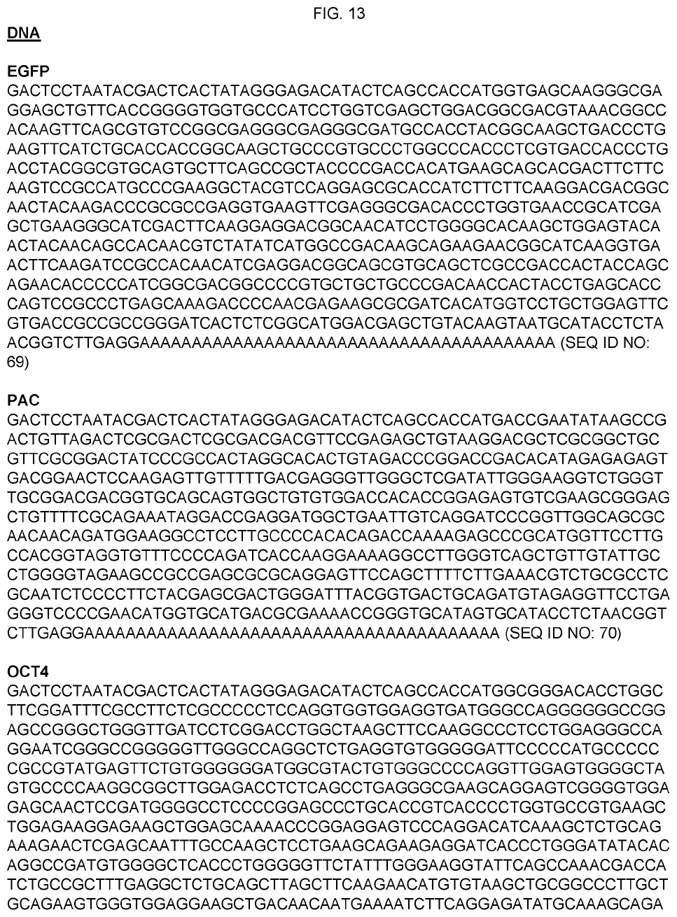

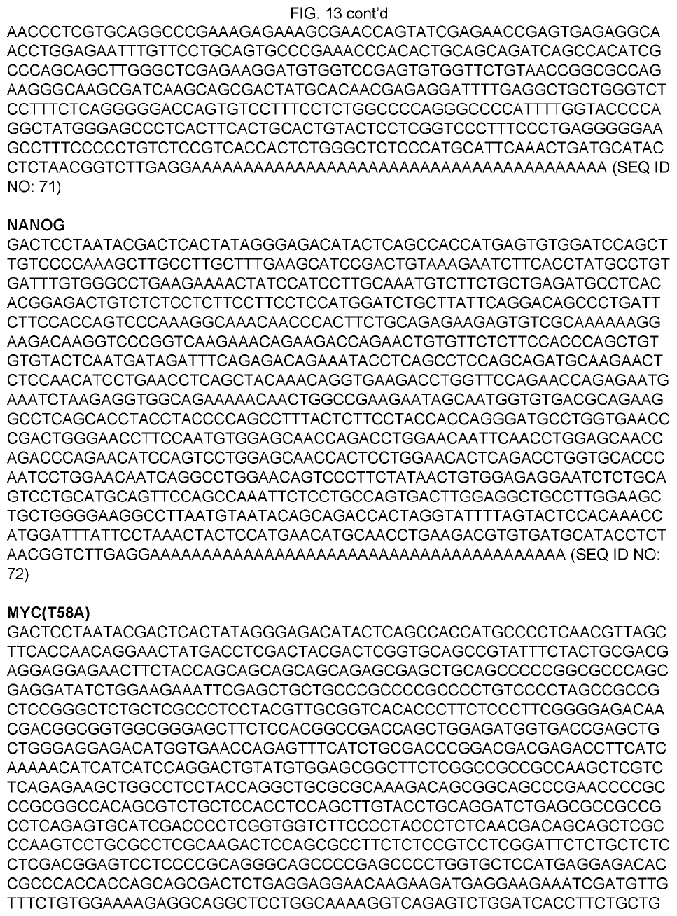

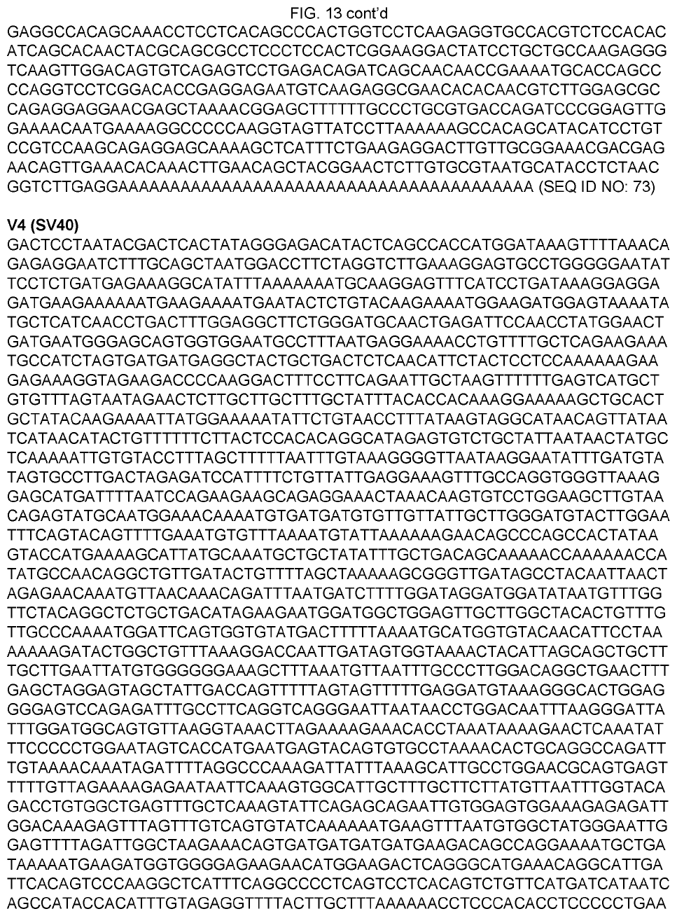

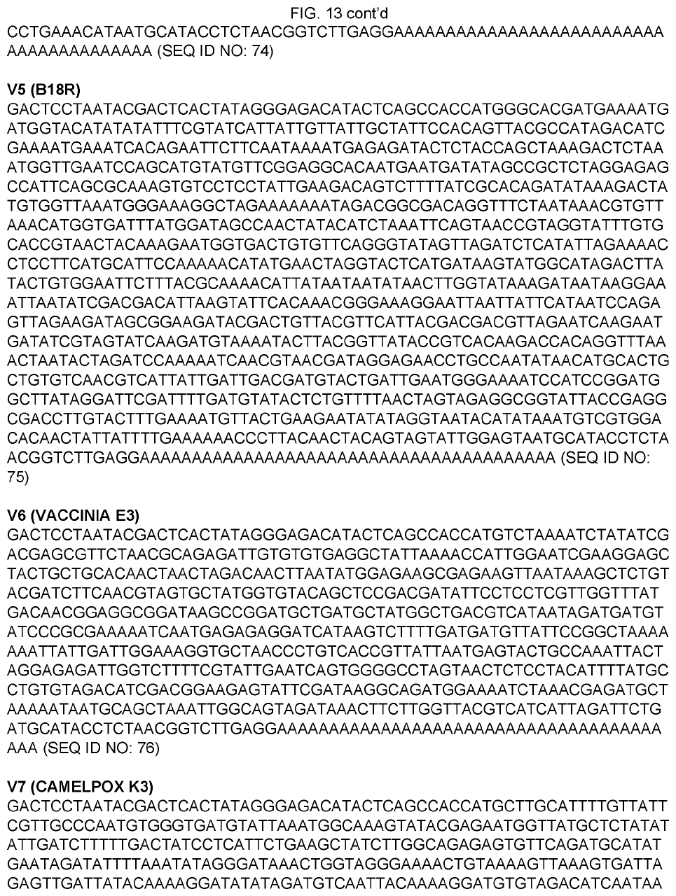

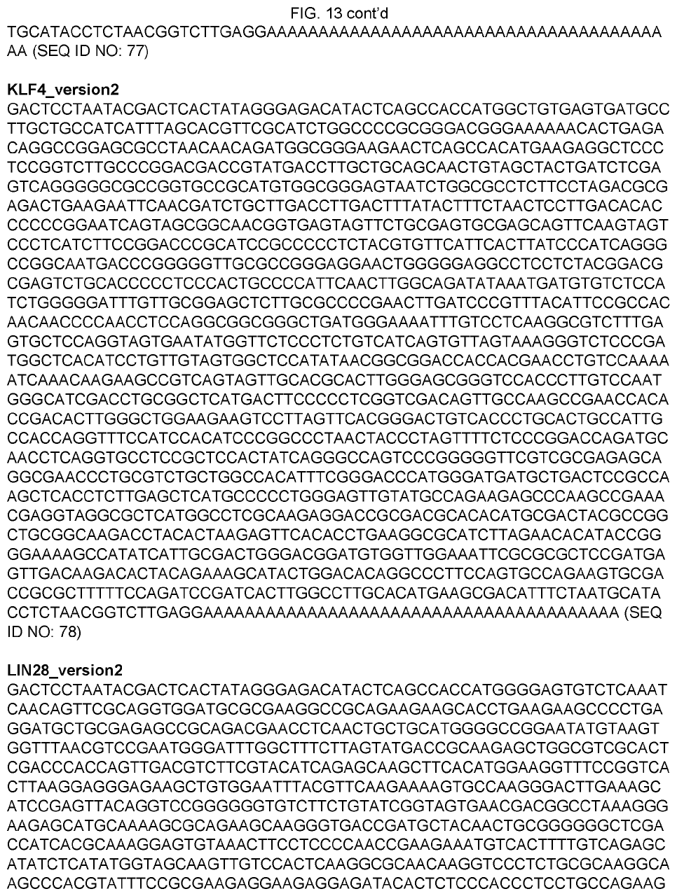

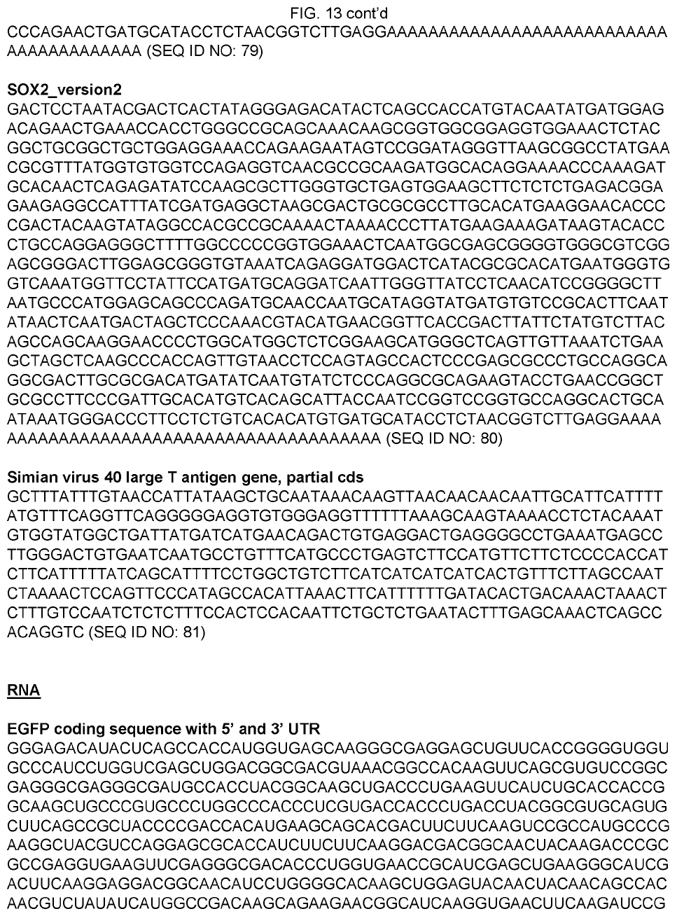

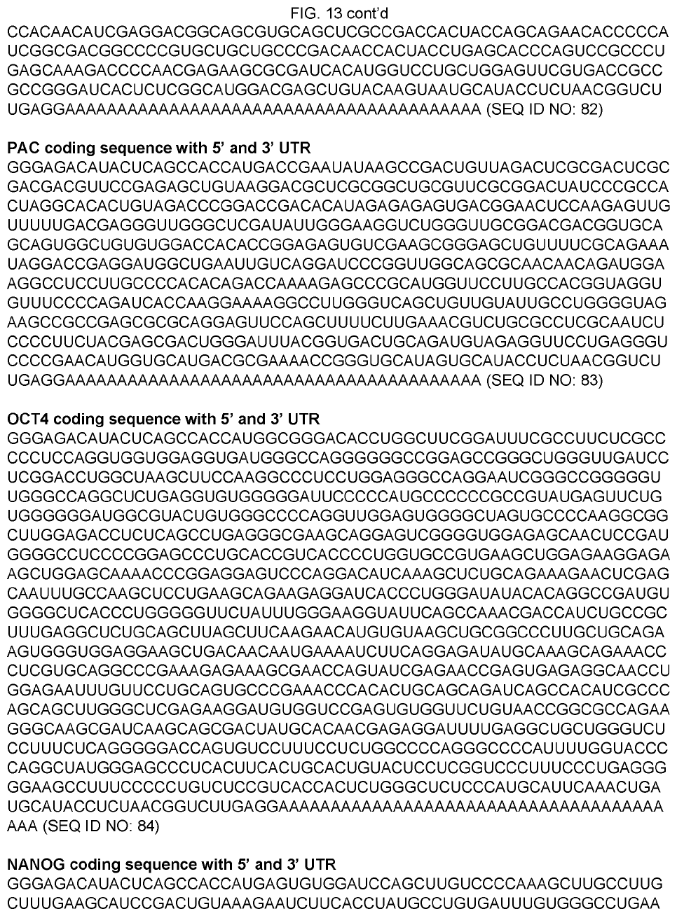

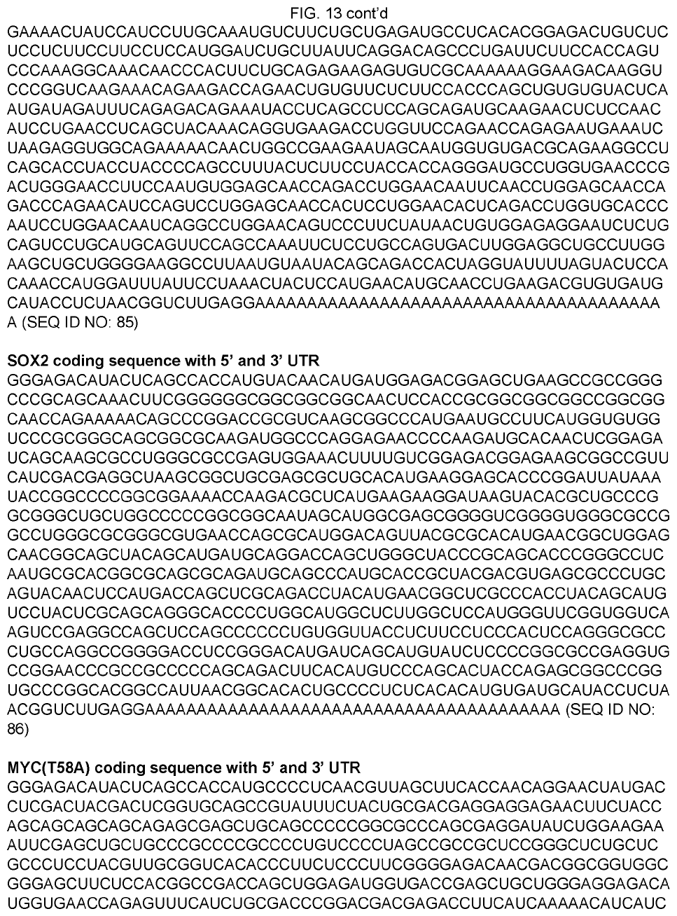

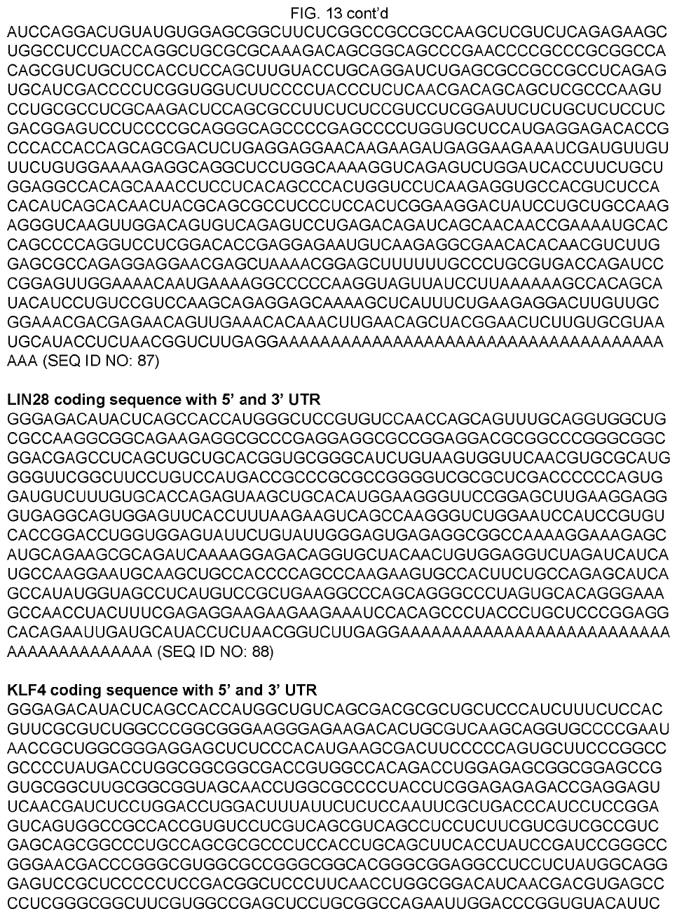

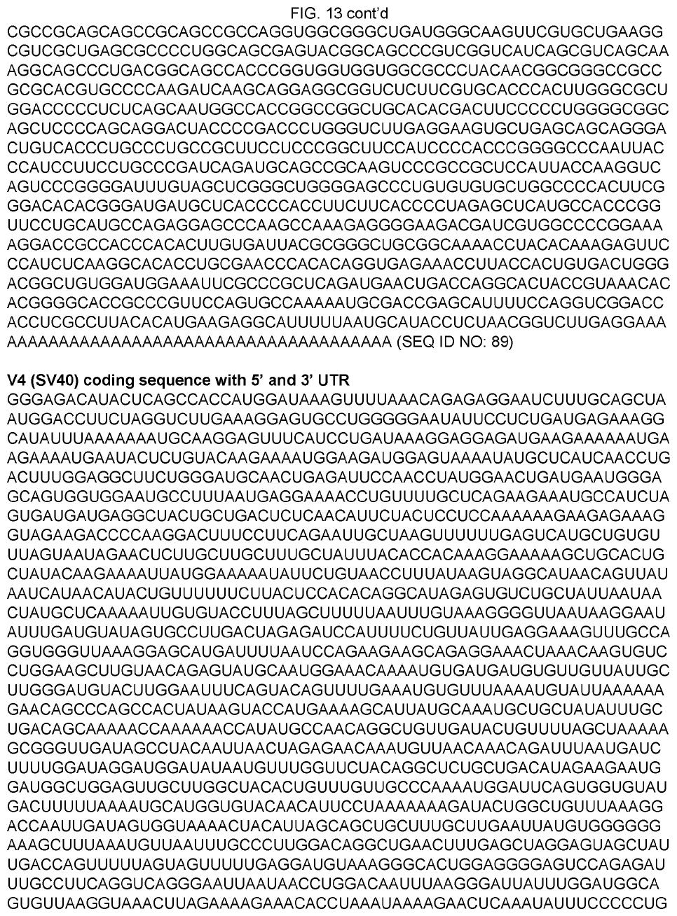

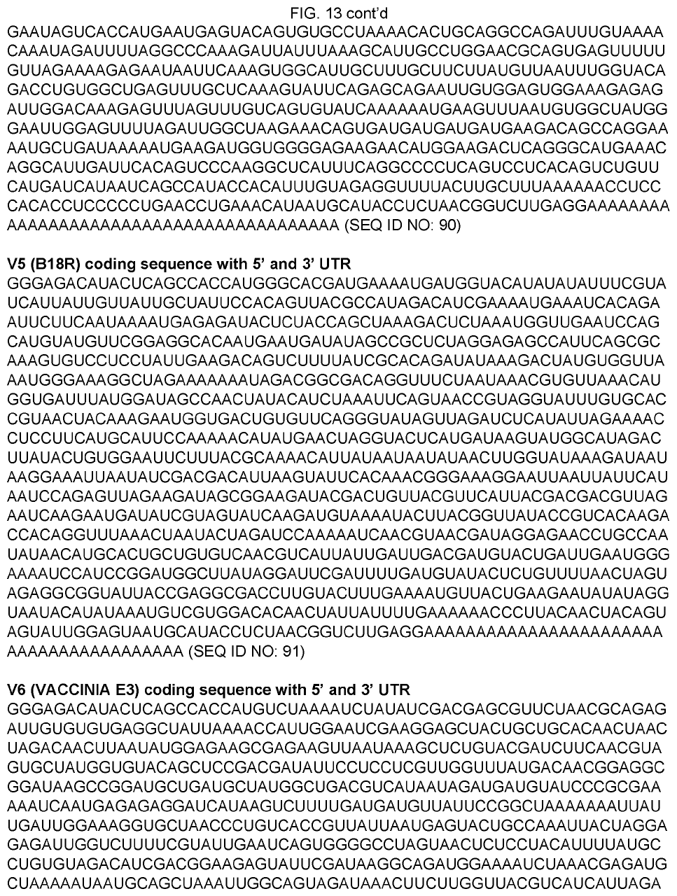

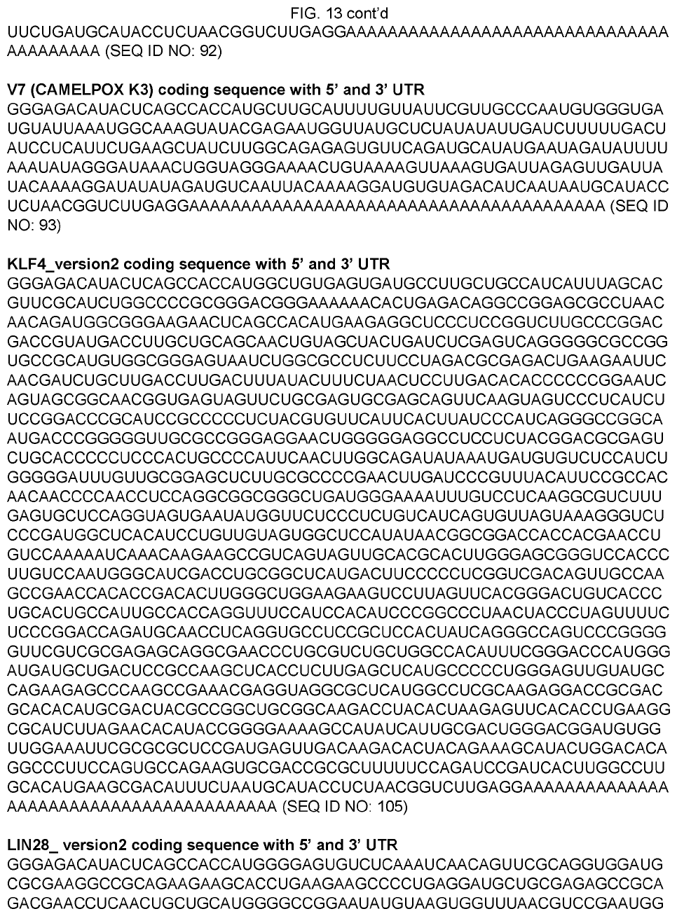

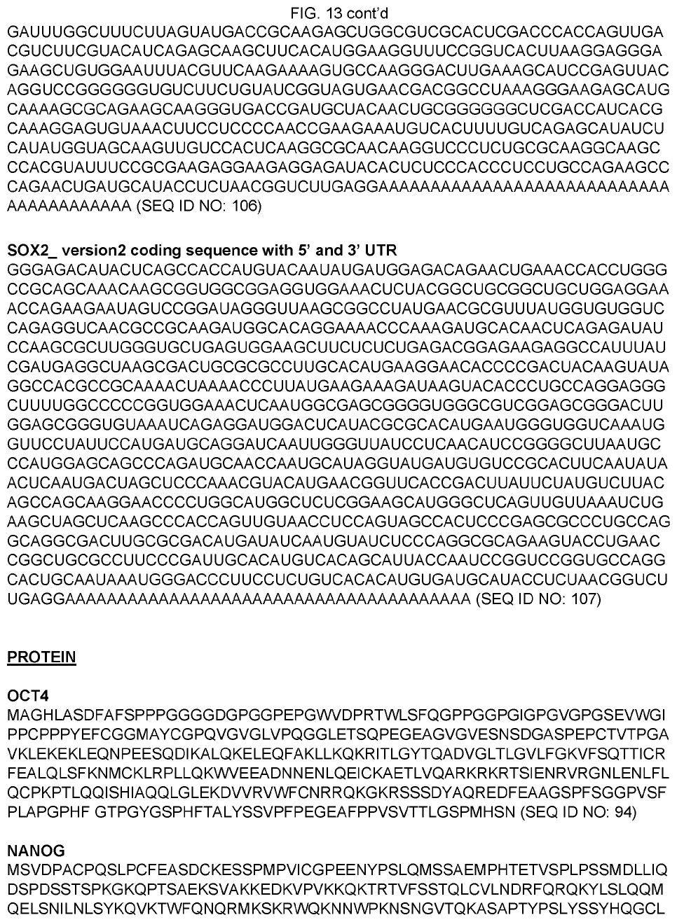

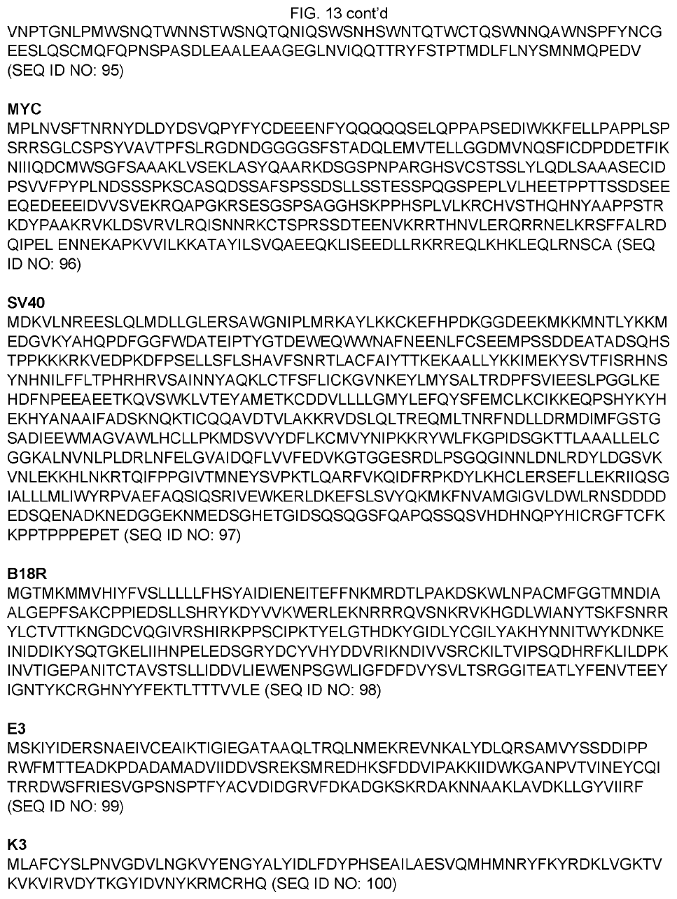

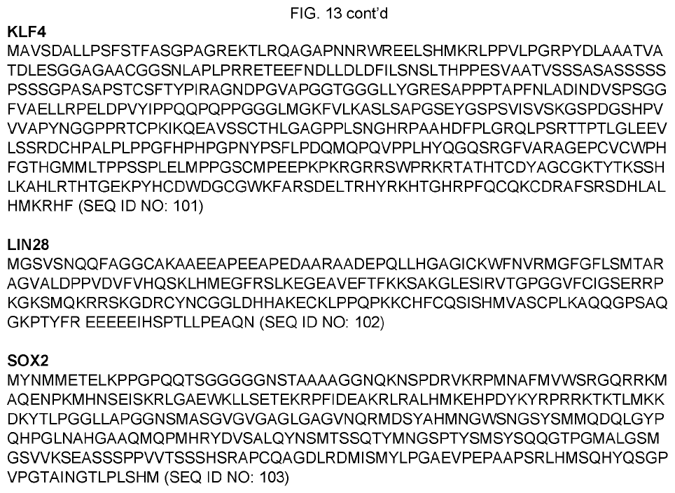

An “Oct polypeptide” refers to the Octamer family of transcription factors. Exemplary Oct polypeptides includes, Oct-1, Oct-2, Oct-3/4, Oct-6, Oct-7, Oct-8, Oct-9, and Oct-11. e.g. Oct3/4 (referred to herein as “Oct4”) contains the POU domain, a 150 amino acid sequence conserved among Pit-1, Oct-1, Oct-2, and uric-86. See, Ryan, A. K. & Rosenfeld, M. G. Genes Dev. 11, 1207-1225 (1997). In some embodiments, variants have at least 85%, 90%, or 95% amino acid sequence identity across their whole sequence compared to a naturally occurring Oct polypeptide family member such as to those listed above or such as listed in Genbank accession number NP002692.2 (human Oct4) or NP038661.1 (mouse Oct4). Oct polypeptides (e.g., Oct3/4) can be from human, mouse, rat, bovine, porcine, or other animals. Oct4 (Octamer-4) is a homeodomain transcription factor of the POU family and regulates the expression of numerous genes (see, e.g., J. Biol. Chem., Vol. 282, Issue 29, 21551-21560, Jul. 20, 2007). DNA and RNA sequences encoding an Oct4 protein are disclosed in FIG. 13. Homologs of human Oct4 are known as set forth in the following accession numbers NP_038661.1 and NM_013633.1 (Mus musculus), NP_001009178 and NM_001009178 (Rattus norvegicus), and NP_571187 and NM_131112 (Danio rerio).

SRY (sex determining region Y)-box 2, also known as SOX2, is a transcription factor that plays a role in self-renewal of undifferentiated embryonic stem cells and transactivation of Fgf4 as well as modulating DNA bending (see, e.g., Scaffidi et al. J. Biol. Chem., Vol. 276, Issue 50, 47296-47302, Dec. 14, 2001). A “Sox polypeptide” refers to members of the SRY-related HMG-box (Sox) transcription factors, characterized by the presence of the high-mobility group (HMG) domain. See, e.g., Dang, D. T., et al., Int. J. Biochem. Cell Biol. 32:1103-1121 (2000). Exemplary Sox polypeptides include, e.g., Sox1, Sox2, Sox3, Sox4, Sox5, Sox6, Sox7, Sox8, Sox9, Sox10, Sox11, Sox12, Sox13, Sox14, Sox15, Sox17, Sox18, Sox-21, and Sox30. Sox1 has been shown to yield iPSC with a similar efficiency as Sox2, and genes Sox3, Sox15, and Sox18 have also been shown to generate iPSC, although with somewhat less efficiency than Sox2. See, Nakagawa, et al., Nature Biotechnology 26:101-106 (2007). In some embodiments, a naturally occurring Sox polypeptide family member includes the sequence as listed in Genbank accession number CAA83435 (human Sox2). Sox polypeptides (e.g., Sox1, Sox2, Sox3, Sox15, or Sox18) can be from human, mouse, rat, bovine, porcine, or other animals. DNA and RNA sequences encoding a Sox2 protein are set forth in in FIG. 13. Homologs of human Sox2 are known.

Kruppel-like factor 4, also known as KLF4 plays a role in stem cell maintenance and growth. A “Klf polypeptide” refers to members of the family of Kruppel-like factors (Klfs), zinc-finger proteins that contain amino acid sequences similar to those of the Drosophila embryonic pattern regulator Kruppel. See, Dang, D. T., Pevsner, J. & Yang, V. W., Cell Biol. 32, 1103-1121 (2000). Exemplary Klf family members include, Klf1, Klf2, Klf3, Klf-4, Klf5, Klf6, Klf7, Klf8, Klf9, Klf10, Klf11, Klf12, Klf13, Klf14, Klf15, Klf16, and Klf17. Klf2 and Klf-4 were found to be factors capable of generating iPSC in mice, and related genes Klf1 and Klf5 did as well, although with reduced efficiency. See, Nakagawa, et al., Nature Biotechnology 26:101-106 (2007). In some embodiments, a naturally occurring Klf polypeptide family member includes the sequence as listed in Genbank accession number CAX16088 (mouse Klf4) or CAX14962 (human Klf4). Klf polypeptides (e.g., Klf1, Klf4, and Klf5) can be from human, mouse, rat, bovine, porcine, or other animals. To the extent a Klf polypeptide is described herein, it can be replaced with an estrogen-related receptor beta (Essrb) polypeptide. Thus, it is intended that for each Klf polypeptide embodiment described herein, a corresponding embodiment using Essrb in the place of a Klf4 polypeptide is equally described. DNA and RNA sequences encoding a KLF4 protein are disclosed in FIG. 13. Homologs of human KLF4 are known and include NP_034767, NM_010637 (Mus musculus).

Nanog is a gene expressed in embryonic stem cells (ESCs) and plays a role in maintaining pluripotency. Nanog is thought to function with Sox2. DNA and RNA sequences encoding a Nanog protein are set forth in FIG. 13. Human Nanog protein (see, e.g., Accession number NP_079141) is a 305 amino acid protein with a homeodomain motif that is localized to the nuclear component of cells. Similar to murine Nanog, N-terminal region of human Nanog is rich in Ser, Thr and Pro residues and the C-terminus includes Trp repeats. The homeodomain in human Nanog ranges from residue 95 to residue 155. Homologs of human Nanog are known.

The protein LIN28, also known as CSDD1 and ZCCHC1, encoded by the Lin28 gene, is a RNA-binding protein. The locus of the gene encoding LIN28 is found on Chromosome 1 p36.11. LIN28 promotes translation of certain mRNAs by binding to them and it is highly expressed in human embryonic stem cells. Lin28 can be used as one of the four factors used in reprogramming somatic cells to induced pluripotent stem cells. The three other transcription factors used are Oct3/4, Sox2 and Klf4. A human Lin28 (RNA: NM_024674; protein: NP_078950) is set forth in in FIG. 13.

The MYC family of cellular genes includes c-myc, N-myc, and L-myc, three genes that function in regulation of cellular proliferation, differentiation, and apoptosis (Henriksson and Luscher 1996; Facchini and Penn 1998). A “Myc polypeptide” refers to members of the Myc family (see, e.g., Adhikary, S. & Eilers, M. Nat. Rev. Mol. Cell Biol. 6:635-645 (2005)). Exemplary Myc polypeptides include, e.g., c-Myc, N-Myc and L-Myc. In some embodiments, a naturally occurring Myc polypeptide family member includes the sequence listed in Genbank accession number CAA25015 (human Myc). Myc polypeptides (e.g., c-Myc) can be from human, mouse, rat, bovine, porcine, or other animals. Although myc family genes have common structural and biological activity. N-Myc is a member of the MYC family and encodes a protein with a basic helix-loop-helix (bHLH) domain. The genomic structures of c-myc and N-myc are similarly organized and are included of three exons. Most of the first exon and the 3′ portion of the third exon contain untranslated regions that carry transcriptional or post-transcriptional regulatory sequences. N-myc protein is found in the nucleus and dimerizes with another bHLH protein in order to bind DNA. DNA and RNA sequences encoding a c-Myc protein are set forth in FIG. 13. Homologs and variants of the Myc family of proteins are known in the art.

The large T antigen from Simian Vacuolating Virus 40 (SV40Tag) is a hexamer protein that is a dominant-acting oncoprotein and capable of inducing malignant transformation of a variety of cell types. More specifically, the SV40 T-antigen binds and inactivates tumor suppressor proteins (p53, p105-Rb). This causes the cells to leave G1 phase and enter into S phase, which promotes DNA replication. Because the SV40 T-antigen function is similar to Myc, it can replace the function and role of Myc as one of the RF. The sequence of SV40 T-antigen is set forth in in FIG. 13.

Each of the RFs also include any of the naturally-occurring members of the family, or variants thereof that maintain RF activity, similar (within at least 50%, 80%, or 90% activity) compared to the closest related naturally occurring family member, or polypeptides including at least the DNA-binding domain of the naturally occurring family member, and can further include a transcriptional activation domain. Generally, the same species of protein will be used with the species of cells being manipulated. In some embodiments, variants have at least 85%, 90%, or 95% amino acid sequence identity across their whole sequence compared to a naturally occurring polypeptide family member.

The disclosure provides methods and compositions for generating iPSC from somatic cells (e.g., fibroblast cells, CD34+ cells, and mesenchymal stem cells). The compositions and methods include the use of EEC as disclosed herein. The EEC include an RNA sequence encoding at least one RF, which induce reprogramming of somatic cells to iPSC.

In any of the foregoing embodiments, the RF include variants and degenerate polynucleotide sequences. For example, a RF can include homologs and variants of an OCT-4 polypeptide, KLF polypeptide, SOX polypeptide, MYC polypeptide, Nanog polypeptide, SV40Tag polypeptide, or Lin28 polypeptide. For example, a RF coding sequence for Nanog useful in any of the EEC embodiments described herein can include (i) a polynucleotide encoding a polypeptide as disclosed in FIG. 13 (or a polynucleotide including at least 95% identity thereto and which encodes a polypeptide having Nanog activity; (ii) a polynucleotide having a sequence as disclosed in FIG. 13 or (iii) a polynucleotide encoding a polypeptide as disclosed in FIG. 13 containing 1 to 10 conservative amino acid substitutions and wherein the polypeptide has Nanog activity; and wherein any of the foregoing nucleic acid sequences can have “T” replaced with “U”. For example, a RF coding sequence for Oct4 useful in any of the EEC embodiments described herein can include (i) a polynucleotide encoding Oct4 as disclosed in FIG. 13 (or a polynucleotide including at least 95% identity thereto and which encodes a polypeptide having Oct4 activity; (ii) a polynucleotide having a sequence as disclosed in FIG. 13, or (iii) a polynucleotide encoding a polypeptide as disclosed in FIG. 13, containing 1 to 10 conservative amino acid substitutions and wherein the polypeptide has Oct4 activity; and wherein any of the foregoing nucleic acid sequences can have “T” replaced with “U”. For example, a RF coding sequence for SOX (e.g., Sox2) useful in any of the EEC embodiments described herein can include (i) a polynucleotide encoding a SOX (e.g., Sox2) polypeptide as disclosed in FIG. 13 (or a polynucleotide including at least 95% identity thereto and which encodes a polypeptide having SOX (e.g., Sox2) activity; (ii) a polynucleotide having a sequence as disclosed in FIG. 13 or (iii) a polynucleotide encoding a polypeptide as disclosed in FIG. 13 containing 1 to 10 conservative amino acid substitutions and wherein the polypeptide has SOX (e.g., Sox2) activity; and wherein any of the foregoing nucleic acid sequences can have “T” replaced with “U”. For example, a RF coding sequence for KLF (e.g., KLF4) useful in any of the EEC embodiments described herein can include (i) a polynucleotide encoding a KLF (e.g., KLF4) polypeptide as disclosed in FIG. 13 (or a polynucleotide including at least 95% identity thereto) and which encodes a polypeptide having KLF (e.g., KLF4) activity; (ii) a polynucleotide having a sequence as disclosed in FIG. 13 or (iii) a polynucleotide encoding a KLF (e.g., KLF4) polypeptide as disclosed in FIG. 13 containing 1 to 10 conservative amino acid substitutions and wherein the polypeptide has KLF (e.g., KLF4) activity; and wherein any of the foregoing nucleic acid sequences can have “T” replaced with “U”. For example, a RF coding sequence for Lin28 useful in any of the EEC embodiments described herein can include (i) a polynucleotide encoding Lin28 as disclosed in FIG. 13 (or a polynucleotide including at least 95% identity thereto and which encodes a polypeptide having Lin28 activity; (ii) a LIN28polynucleotide as disclosed in FIG. 13 or (iii) a polynucleotide encoding LIN28 as disclosed in FIG. 13 containing 1 to 10 conservative amino acid substitutions and wherein the polypeptide has LIN28 activity; and wherein any of the foregoing nucleic acid sequences can have “T” replaced with “U”. For example, a RF coding sequence for Myc (e.g., c-Myc) useful in any of the EEC embodiments described herein can include (i) a polynucleotide encoding a polypeptide as disclosed in FIG. 13 (or a polynucleotide including at least 95% identity thereto and which encodes a polypeptide having MYC activity); (ii) a polynucleotide as disclosed in FIG. 13 or (iii) a polynucleotide encoding a MYC (e.g., cMYC) polypeptide as disclosed in FIG. 13 containing 1 to 10 conservative amino acid substitutions and wherein the polypeptide has MYC activity; and wherein any of the foregoing nucleic acid sequences can have “T” replaced with “U”. For example, a RF coding sequence for SV40Tag useful in any of the EEC embodiments described herein can include (i) a polynucleotide encoding a polypeptide as disclosed in FIG. 13 (or a polynucleotide including at least 95% identity thereto and which encodes a polypeptide having SV40tag activity); (ii) a polynucleotide as disclosed in FIG. 13 or (iii) a polynucleotide encoding a SV40tag polypeptide as disclosed in FIG. 13 containing 1 to 10 conservative amino acid substitutions and wherein the polypeptide has SV40tag activity; and wherein any of the foregoing nucleic acid sequences can have “T” replaced with “U”.

cDNA coding for the human Oct4 (pour5f1), Sox, Klf, Myc (c-Myc, n-Myc, or L-Myc), SV40Tag, Lin28 and Nanog, variants and homologs thereof can be cloned and expressed using techniques known in the art. Using the sequences set forth herein polynucleotides encoding one or more de-differentiation factors can be cloned into a suitable vector for expression in a cell type of interest.

A RF “activity” (e.g., a RF variant activity) refers to the ability to de-differentiate a somatic cell when expressed in combination with other RF as known in the art. For example, an Oct4 variant can be measured for Oct4 activity by co-expressing the Oct4 variant in a somatic cell with klf, Sox and Lin28 and determining if a somatic cell de-differentiates. If the cell de-differentiates than the Oct4 variant can be said to have Oct4 activity.

In some embodiments, at least one of Oct4, Klf, Sox and Lin28 is used to reprogram somatic cells and produce iPSC. In other embodiments, at least Oct-4 and Sox are used to reprogram somatic cells and iPSC. In other embodiments, at least Klf is used in combination with Oct4 and Sox to reprogram somatic cells and produce iPSC. In other embodiments, at least Lin28 is used in combination with Oct4 and Sox to reprogram somatic cells and produce iPSC. In particular embodiments, any combination of RFs can be used to reprogram somatic cells and produce iPSC.

In one embodiment, one EEC is utilized to express one of the RF. In another embodiment, more than one EEC is used to express at least one RF on each EEC. In yet another embodiment, more than two EEC are used to express at least one RF on each EEC. In yet another embodiment, more than three EEC are used to express at least one RF on each EEC. In certain embodiments, each EEC includes one or more coding sequences for factors that induce a somatic cell to become an iPSC, wherein the combination of the more than one EEC includes all the coding sequences for all RF necessary for inducing de-differentiation into an iPSC.

In more specific embodiments, an EEC includes coding sequences for expression of Oct4, Sox, KLF, Myc, SV40Tag, LIN28 and/or Nanog. In a specific embodiment, one EEC includes a coding sequence for Oct4 and a second EEC includes a coding sequence for Sox.

In one embodiment, at least three EEC constructs are utilized: one EEC including a coding sequence for Oct4, a second EEC including a coding sequence for SOX, and a third EEC including a coding sequence for at least one of KLF, MYC, Lin28, SV40Tag and/or Nanog. In another embodiment, at least four EEC constructs are utilized: one EEC including a coding sequence for Oct4, a second EEC including a coding sequence for Sox, a third EEC including a coding sequence for Nanog, and a fourth EEC encoding at least one of KLF, MYC, SV40Tag, and/or LIN28. In still another embodiment, at least five EEC constructs are utilized: one EEC including a coding sequence for Oct4, a second EEC including a coding sequence for Sox, a third EEC including a coding sequence for Nanog, a fourth EEC including a coding sequence for KLF and a fifth EEC encoding at least one of MYC or SV40Tag and/or LIN28. In yet another embodiment, at least five EEC constructs are utilized: one EEC including a coding sequence for Oct4, a second EEC including a coding sequence for Sox, a third EEC including a coding sequence for Nanog, a fourth EEC including a coding sequence for KLF, and a fifth EEC encoding LIN28. In another embodiment, at least six EEC constructs are utilized: one EEC including a coding sequence for Oct4, a second EEC including a coding sequence for Sox, a third EEC including a coding sequence for Nanog, a fourth EEC including a coding sequence for KLF, a fifth EEC encoding LIN28, and a sixth encoding either MYC or the SV40Tag.

In particular embodiments, one EEC can include a coding sequence for more than one RF. For example, one EEC can include a coding sequence for Oct 4, Sox, KLF, Myc, SV40Tag, LIN28 and/or Nanog. In particular embodiments, a first EEC can include a coding sequence for Sox, KLF, and/or Myc; and a second EEC can include a coding sequence for SV40Tag, LIN28 and/or Nanog. Any combination of RF can be distributed among any number of EEC.

As described herein, EEC increase expression of one or more RF. This increase can be in relation to natural expression levels of one or more RF, when compared to coding sequences that do not include the mini-enhancer sequence in the 5′ UTR, when compared to coding sequences that do not include the stem loop structure in the 3′ UTR, when compared to coding sequences that do not include the mini-enhancer sequence in the 5′ UTR and the stem loop structure in the 3′ UTR, when compared to coding sequences that contain modified nucleotides but not the EEC disclosed herein, and/or in relation to how a protein has been historically or conventionally expressed. In certain examples, the increased RF expression includes at least 10% more RF expression, at least 20% more RF expression, at least 30% more RF expression, at least 40% more RF expression, at least 50% more RF expression, at least 60% more RF expression, at least 70% more RF expression, at least 80% more RF expression, at least 90% more RF expression, at least 100% more RF expression, at least 200% more RF expression, at least 300% more RF expression as compared to a relevant control system or condition. In particular embodiments, the relevant control system or condition includes the RF expression of cells that are not transfected with EEC.

Those of skill in the art will recognize that, due to the degenerate nature of the genetic code, a variety of codons differing in their nucleotide sequences can be used to encode a given amino acid. A particular polynucleotide or gene sequence encoding a polypeptide described herein are referenced merely to illustrate an embodiment of the disclosure, and the disclosure includes polynucleotides of any sequence that encode a polypeptide including the same amino acid sequence of the polypeptides and proteins of the enzymes utilized in the methods of the disclosure. In a similar fashion, a polypeptide can typically tolerate one or more amino acid substitutions, deletions, and insertions in its amino acid sequence without loss or significant loss of a desired activity. The disclosure includes such polypeptides with alternate amino acid sequences, and the amino acid sequences encoded by the RNA or DNA sequences shown herein merely illustrate embodiments of the disclosure.

RF variants can also be expressed. RF variants refer to RF which differ in their amino acid sequence from a native or reference RF sequence. The amino acid sequence variants may possess substitutions, deletions, and/or insertions at certain positions within the amino acid sequence, as compared to a native or reference sequence. Ordinarily, variants will possess at least 50% sequence identity to a native or reference sequence. In particular embodiments, variant include at least 80%, at least 85%, at least 90%, at least 95%, at least 99% sequence identity to a native or reference sequence.

Homologous protein sequences are those with a common evolutionary origin. There are two types of protein homologs, depending on how they originated: paralogs, derived from a gene duplication event, and orthologs, originated from a speciation event. A protein has “homology” or is “homologous” to a second protein if the nucleic acid sequence that encodes the protein has a similar sequence to the nucleic acid sequence that encodes the second protein. Alternatively, a protein has homology to a second protein if the two proteins have “similar” amino acid sequences. (Thus, the term “homologous proteins” is defined to mean that the two proteins have similar amino acid sequences).

As used herein, two proteins (or a region of the proteins) are substantially homologous when the amino acid sequences have at least 30%, 40%, 50% 60%, 65%, 70%, 75%, 80%, 85%, 90%, 91%, 92%, 93%, 94%, 95%, 96%, 97%, 98%, or 99% identity. To determine the percent identity of two amino acid sequences, or of two nucleic acid sequences, the sequences are aligned for optimal comparison purposes (e.g., gaps can be introduced in one or both of a first and a second amino acid or nucleic acid sequence for optimal alignment and non-homologous sequences can be disregarded for comparison purposes). In one embodiment, the length of a reference sequence aligned for comparison purposes is at least 30%, typically at least 40%, more typically at least 50%, even more typically at least 60%, and even more typically at least 70%, 80%, 90%, 100% of the length of the reference sequence. The amino acid residues or nucleotides at corresponding amino acid positions or nucleotide positions are then compared. When a position in the first sequence is occupied by the same amino acid residue or nucleotide as the corresponding position in the second sequence, then the molecules are identical at that position (as used herein amino acid or nucleic acid “identity” is equivalent to amino acid or nucleic acid “homology”). The percent identity between the two sequences is a function of the number of identical positions shared by the sequences, taking into account the number of gaps, and the length of each gap, which need to be introduced for optimal alignment of the two sequences.

When “homologous” is used in reference to proteins or peptides, it is recognized that residue positions that are not identical often differ by conservative amino acid substitutions. A “conservative amino acid substitution” is one in which an amino acid residue is substituted by another amino acid residue having a side chain (R group) with similar chemical properties (e.g., charge or hydrophobicity). In general, a conservative amino acid substitution will not substantially change the functional properties of a protein. In cases where two or more amino acid sequences differ from each other by conservative substitutions, the percent sequence identity or degree of homology may be adjusted upwards to correct for the conservative nature of the substitution. Means for making this adjustment are well known to those of skill in the art (see, e.g., Pearson et al., 1994).

A “conservative amino acid substitution” is one in which the amino acid residue is replaced with an amino acid residue having a similar side chain. Families of amino acid residues having similar side chains have been defined in the art. These families include amino acids with basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine), nonpolar side chains (e.g., alanine, valine, leucine, isoleucine, proline, phenylalanine, methionine, tryptophan), beta-branched side chains (e.g., threonine, valine, isoleucine) and aromatic side chains (e.g., tyrosine, phenylalanine, tryptophan, histidine). The following six groups each contain amino acids that are conservative substitutions for one another:

1) Serine(S), Threonine (T); 2) Aspartic Acid (D), Glutamic Acid (E); 3) Asparagine (N), Glutamine (Q); 4) Arginine (R), Lysine (K); 5) Isoleucine (I), Leucine (L), Methionine (M), Alanine (A), Valine (V), and 6) Phenylalanine (F), Tyrosine (Y), Tryptophan (W).

Sequence homology for polypeptides, which can also be referred to as percent sequence identity, is typically measured using sequence analysis software. See, e.g., the Sequence Analysis Software Package of the Genetics Computer Group (GCG), University of Wisconsin Biotechnology Center, 910 University Avenue, Madison, Wis. 53705. Protein analysis software matches similar sequences using measure of homology assigned to various substitutions, deletions and other modifications, including conservative amino acid substitutions. For instance, GCG contains programs such as “Gap” and “Bestfit” which can be used with default parameters to determine sequence homology or sequence identity between closely related polypeptides, such as homologous polypeptides from different species of organisms or between a wild type protein and a mutein thereof. See, e.g., GCG Version 6.1.

A typical algorithm used comparing a molecule sequence to a database containing a large number of sequences from different organisms is the computer program BLAST (Altschul, 1990; Gish, 1993; Madden, 1996; Altschul, 1997; Zhang, 1997), especially blastp or tblastn (Altschul, 1997). Typical parameters for BLASTp are: Expectation value: 10 (default); Filter: seg (default); Cost to open a gap: 11 (default); Cost to extend a gap: 1 (default); Max. alignments: 100 (default); Word size: 11 (default); No. of descriptions: 100 (default); Penalty Matrix: BLOWSUM62.

When searching a database containing sequences from a large number of different organisms, it is typical to compare amino acid sequences. Database searching using amino acid sequences can be measured by algorithms other than blastp known in the art. For instance, polypeptide sequences can be compared using FASTA, a program in GCG Version 6.1. FASTA provides alignments and percent sequence identity of the regions of the best overlap between the query and search sequences (Pearson, 1990). For example, percent sequence identity between amino acid sequences can be determined using FASTA with its default parameters (a word size of 2 and the PAM250 scoring matrix), as provided in GCG Version 6.1.

### (iii) STABILIZING EEC

It has been previously attempted to stabilize IVT-RNA by various modifications in order to achieve higher and prolonged expression of transferred IVT-RNA. However, despite the success of RNA transfection-based strategies to express peptides and proteins in cells, there remain issues related to RNA stability, sustained expression of the encoded peptide or protein and cytotoxicity of the RNA. For example, it is known that exogenous single-stranded RNA activates defense mechanisms in mammalian cells.

Several groups have suggested that due to the activated defense mechanisms, to achieve a high enough level of protein expression from IVT-RNA transfected into cells, the mRNA transcript must either contain modified nucleotides (e.g. U.S. Pat. No. 9,750,824) or additional reagents in the form of protein or IVT-RNA that include immune evading factors (IEF) (e.g. U.S. Pat. No. 10,207,009) An IEF or “immune evading factor” refers to a molecule (e.g., protein) that causes a cell to be recognized less by the immune system. In particular embodiments, IEF include viral genes encoding proteins that dampen the cellular immune response by, for example, preventing engagement of the IFN receptor by extracellular IFN (e.g., B18R from vaccinia virus), by inhibiting intracellular IFN signaling (e.g., E3 and K3 both from vaccinia virus) or by working in both capacities (e.g., NS1 from influenza) (Liu et al., Sci Rep 9:11972, 2019). In particular embodiments, IEF include B18R, E3, K3, NS1, or ORF8 (from SARS-COV2). In particular embodiments, B18R [Vaccinia virus] includes the sequence as set forth in GenBank: CAA01478.1, E3 [Vaccinia virus] includes the sequence as set forth in GenBank: UZL86786.1, and K3 [Vaccinia virus] includes the sequence as set forth in GenBank: UZL86760.1. In particular embodiments, B18R [Vaccinia virus] is encoded by the sequence as set forth in GenBank: A19579.1, E3 [Vaccinia virus] is encoded by the ORF2 sequence within the sequence as set forth in GenBank: M36339.1, and K3 [Vaccinia virus] is encoded by the ORF K3 sequence with the sequence as set forth in GenBank: D00382.1.

Certain aspects of the current disclosure were designed to overcome the activated defense mechanisms by introducing secondary and tertiary structures into the EEC, instead of using modified nucleotides, microRNAs, or IEF. According to further embodiments, particular embodiments do not use modified nucleotides to express RF or IEF and/or do not use microRNAs. Further embodiments do not use modified nucleotides or microRNAs to prolong the translation of from IVT-RNA transfected into cells or for any other purpose.

MicroRNAs (or miRNA) are 19-25 nucleotide long noncoding RNAs that bind to the 3′ UTR of nucleic acid molecules and down-regulate gene expression either by reducing nucleic acid molecule stability or by inhibiting translation. In particular embodiments, the EEC do not include any known microRNA target sequences, microRNA sequences, or microRNA seeds.

A microRNA sequence includes a “seed” region, i.e., a sequence in the region of positions 2-8 of the mature microRNA, which sequence has perfect Watson-Crick complementarity to the miRNA target sequence.

Modified NTPs are those that have additional chemical groups attached to them to modify their chemical structure. Examples of these modified NTPs include: pseudouridine, methylpseudouridine, methyluridine (m5U), N1-methyl-pseudouridine, 5-methoxyuridine (mo5U), and 2-thiouridine (s2U). 5′ caps are not modified NTPs.

In particular embodiments, EEC specifically exclude microRNA. In particular embodiments, EEC specifically exclude modified NTPs. In particular embodiments, EEC specifically exclude IEF coding sequences.

In particular embodiments, IEF-encoding EEC are added to cell media during reprogramming. In particular embodiments, microRNA is added to the cell media during reprogramming. In particular embodiments, IEF protein is added to cell media during reprogramming.

### (iv) IN VITRO SYNTHESIS OF EEC

The process of mRNA production may include in vitro transcription, cDNA template removal and RNA clean-up, and mRNA capping and/or tailing reactions.

During in vitro transcription, cDNA from a desired construct is produced according to techniques well known in the art. This given, cDNA may be transcribed using an in vitro transcription (IVT) system. This IVT may allow for in vitro synthesized mRNA of disclosed EEC. The system typically includes a transcription buffer, nucleotides (e.g., NTPs), a polymerase, and modifying enzymes. An RNase inhibitor and other components like pyrophosphatase may be added to the IVT reaction to help improve RNA yield. The NTPs may be manufactured in house, may be selected from a supplier, or may be synthesized as known in the art. The NTPs are selected from naturally occurring NTPs. The polymerase may be selected from T7 RNA polymerase, T3 RNA polymerase, SP6 RNA polymerase and mutant polymerases such as polymerases able to incorporate modified nucleic acids. Modifying enzymes can be used for 5′ capping and 3′ poly-A tailing.

### (v) CELL TYPES

As described herein, somatic cells are reprogrammed to form stem cells (e.g., induce the formation of stem cells) or iPSC.

Stem cells are cells capable of differentiation into other cell types, including those having a particular, specialized function (e.g., tissue specific cells, parenchymal cells and progenitors thereof). There are various classes of stem cells, which can be characterized in their ability to differentiate into a desired cell/tissue type. For example, “progenitor cells” can be either multipotent or pluripotent. Progenitor cells are cells that can give rise to different terminally differentiated cell types, and cells that are capable of giving rise to various progenitor cells. The term “pluripotent” or “pluripotency” refers to cells with the ability to give rise to progeny cells that can undergo differentiation, under the appropriate conditions, into cell types that collectively demonstrate characteristics associated with cell lineages from all of the three germinal layers (endoderm, mesoderm, and ectoderm). Pluripotent stem cells can contribute to all embryonic derived tissues of a prenatal, postnatal or adult animal. A standard art-accepted test, such as the ability to form a teratoma in 8-12 week old SCID mice, can be used to establish the pluripotency of a cell population; however identification of various pluripotent stem cell characteristics can also be used to detect pluripotent cells. “Pluripotent stem cell characteristics” refer to characteristics of a cell that distinguish pluripotent stem cells from other cells. The ability to give rise to progeny that can undergo differentiation, under the appropriate conditions, into cell types that collectively demonstrate characteristics associated with cell lineages from all of the three germinal layers (endoderm, mesoderm, and ectoderm) is a pluripotent stem cell characteristic. Expression or non-expression of certain combinations of molecular markers are also pluripotent stem cell characteristics. For example, human pluripotent stem cells express at least some, and in some embodiments, all of the markers from the following list: SSEA-3, SSEA-4, TRA-1-60, TRA-1-81, TRA-2-49/6E, ALP, Sox2, E-cadherin, UTF-1, Oct4, Rex1, and Nanog. Cell morphologies associated with pluripotent stem cells are also pluripotent stem cell characteristics. In particular embodiments, pluripotency can be verified by reviewing cell morphology, TRA1-60 live staining, performing flow cytometry for pluripotency markers, and/or alkaline phosphatase staining. In particular embodiments, reviewing the morphology of the cell includes looking for colonies with well-defined borders, looking for cells with an enlarged nucleus, and/or looking for cells with a high nucleus to cytosol ratio. In particular embodiments, performing flow cytometry for pluripotency markers includes performing flow cytometry for SSEA-4, Oct4, Nanog, and Sox2.

In comparison, a multipotent stem cell is capable of differentiating into a subset of cells compared to a pluripotent stem cell. For example, a multipotent stem cell may be able to undergo differentiation into one or two of the three germinal layers. As used herein, “non-pluripotent cells” refer to mammalian cells that are not pluripotent cells. Examples of such cells include differentiated cells as well as multipotent cells. Examples of differentiated cells include: cells from a tissue selected from bone marrow, skin, skeletal muscle, fat tissue and peripheral blood. Exemplary cell types include: fibroblasts, hepatocytes, myoblasts, neurons, osteoblasts, osteoclasts, and T-cells.

Another class of cells even more primitive (i.e., uncommitted to a particular differentiation fate) than pluripotent stem cells are the so-called “totipotent” stem cells (e.g., fertilized oocytes, cells of embryos at the two and four cell stages of development), which have the ability to differentiate into any type of cell of the particular species. For example, a single totipotent stem cell could give rise to a complete animal, as well as to any of the myriad of cell types found in the particular species (e.g., humans).

Pluripotent stem cells are a type of cell that undergoes self-renewal while maintaining an ability to give rise to all three germ layer-derived tissues and germ cell lineages. Although pluripotent human embryonic stem (hES) cells derived from human blastocysts are promising sources for cell-based therapies to treat diseases and disorders such as Parkinson's disease, cardiac infarction, spinal cord injury, and diabetes mellitus, their clinical potential has been hampered by their immunogenicity and ethical concerns.

In one embodiment, the disclosure provides cells that are de-differentiated (reprogrammed) to iPSC including characteristics including the ability of self-renewal and differentiation into mesoderm, endoderm and epidermis, wherein the de-differentiated cells can be produced by expression of one or more RF ectopic to the host cell genome using an EEC, as described herein.

The term “precursor cell,” “progenitor cell,” and “stem cell” are used interchangeably in the art refer either to a pluripotent, or lineage-uncommitted, progenitor cell, which is potentially capable of an unlimited number of mitotic divisions to either renew its line or to produce progeny cells which will differentiate into fibroblasts or a lineage-committed progenitor cell and its progeny, which is capable of self-renewal and is capable of differentiating into a parenchymal cell type. Unlike pluripotent stem cells, lineage-committed progenitor cells are generally considered to be incapable of giving rise to numerous cell types that phenotypically differ from each other. Instead, they give rise to one or possibly two lineage-committed cell types.

The type of cell to be reprogrammed, or somatic cell, can be stromal or adherent cells from tissue, blood, or bone marrow. In some embodiments, the somatic cell to be reprogrammed are fibroblasts. In more specific embodiments, the somatic cells to be reprogrammed are human foreskin fibroblasts. In alternative embodiments, the somatic cells to be reprogrammed are human adult fibroblasts.

In other embodiments, the somatic cells to be reprogrammed are derived from bone marrow. In other embodiments, the somatic cells to be reprogrammed are hematopoietic stem cells. Hematopoietic stem cells give rise to different types of blood cells, in lines called myeloid and lymphoid. Myeloid and lymphoid lineages both are involved in dendritic cell formation. Myeloid cells include monocytes, macrophages, neutrophils, basophils, eosinophils, erythrocytes, and megakaryocytes to platelets. Lymphoid cells include T cells, B cells, natural killer cells, and innate lymphoid cells. Hematopoietic stem cells (HSC) possess multipotentiality, enabling them to self-renew and also to produce mature blood cells, such as erythrocytes, leukocytes, platelets, and lymphocytes. CD34 is a marker of human HSC, and all colony-forming activity of human bone marrow (BM) cells is found in the CD34+ fraction. According to certain embodiments, hematopoietic stromal cells are more readily reprogrammed than fully differentiated somatic cells.

In more specific embodiments, the somatic cells to be reprogrammed are CD34+ cells, as determined using antibodies detecting CD34 using techniques described herein.

In other embodiments, the somatic cells to be reprogrammed are derived from the stroma. In other embodiments, the somatic cells to be reprogrammed are mesenchymal stem cells. Mesenchymal stem cells also known as mesenchymal stromal cells or medicinal signaling cells are multipotent stromal cells. Mesenchymal stem cells are more differentiated than pluripotent stem cells, but retain the ability to differentiate into a variety of cell types, including osteoblasts, chondrocytes, myocytes and adipocytes. According to certain embodiments, mesenchymal stromal cells are more readily reprogrammed than fully differentiated somatic cells. In particular embodiments, type of cell to be reprogrammed includes fibroblasts, hepatocytes, myoblasts, neurons, osteoblasts, osteoclasts, kidney cells, immune cells, or stem cells.

### (vi) SOMATIC CELL ISOLATION

The somatic cells to be reprogrammed can be isolated from a sample obtained from a mammalian subject. The subject can be any mammal (e.g., bovine, ovine, porcine, canine, feline, equine, primate), including a human. The sample of cells may be obtained from any of a number of different sources including, for example, bone marrow, skin, foreskin, fetal tissue (e.g., fetal liver tissue), peripheral blood, umbilical cord blood, pancreas and the like. More specifically, the sample of cells may be obtained from the sources above and then purified into one cell type, for example, hematopoietic stem cells, mesenchymal stem cells, or fibroblasts.

The term “isolated” or “purified” when referring to stem cells of the disclosure means cells that are substantially free of cells carrying markers associated with lineage dedication. In particular embodiments, the iPSC are at least 30%, 35%, 40%, 45%, 50%, 55%, 60%, 65%, 70%, 75%, 80%, 85%, 90%, 95% or 99% free of such contaminating cell types. In another embodiment, the isolated stem cells also are substantially free of soluble, naturally occurring molecules. As discussed more fully below, a substantially purified stem cell of the disclosure can be obtained, for example, by extraction (e.g., via density gradient centrifugation and/or flow cytometry) from a culture source. Purity can be measured by any appropriate method. A stem cell of the disclosure can be 99%-100% purified by, for example, flow cytometry (e.g., FACS analysis), as discussed herein. Such purified iPSC will lack any retroviral DNA or retroviral RNA.

Somatic cells (including, adult and neonatal fibroblasts, hematopoietic stem cells, and mesenchymal stem cells) may be readily isolated by disaggregating an appropriate organ or tissue which is to serve as the source of the somatic cells. This may be readily accomplished using techniques known to those skilled in the art. For example, the tissue or organ can be disaggregated mechanically and/or treated with digestive enzymes and/or chelating agents that weaken the connections between neighboring cells making it possible to disperse the tissue into a suspension of individual cells without appreciable cell breakage. Enzymatic dissociation can be accomplished by mincing the tissue and treating the minced tissue with any of a number of digestive enzymes either alone or in combination. These include: trypsin, chymotrypsin, collagenase, elastase, and/or hyaluronidase, DNase, pronase, dispase etc. Mechanical disruption can also be accomplished by a number of methods including the use of grinders, blenders, sieves, homogenizers, pressure cells, or insonators to name but a few. For a review of tissue disaggregation techniques, see Freshney, Culture of Animal Cells. A Manual of Basic Technique, 2d Ed., A.R. Liss, Inc., New York, 1987, Ch. 9, pp. 107-126.

Once the tissue has been reduced to a suspension of individual cells, the suspension can be fractionated into subpopulations from which fibroblasts, mesenchymal stem cells, hematopoietic stem cells and/or other stromal or adherent cells and/or elements can be obtained. This also may be accomplished using standard techniques for cell separation including: cloning and selection of specific cell types, selective destruction of unwanted cells (negative selection), separation based upon differential cell agglutinability in the mixed population, freeze-thaw procedures, differential adherence properties of the cells in the mixed population, filtration, conventional and zonal centrifugation, centrifugal elutriation (counterstreaming centrifugation), unit gravity separation, countercurrent distribution, electrophoresis and fluorescence-activated cell sorting. For a review of clonal selection and cell separation techniques, see Freshney, Culture of Animal Cells. A Manual of Basic Techniques, 2d Ed., A.R. Liss, Inc., New York, 1987, Ch. 11 and 12, pp. 137-168.

The isolation of fibroblasts (either adult or neonatal) may, for example, be carried out as follows: fresh tissue samples are thoroughly washed and minced in Hanks balanced salt solution (HBSS) in order to remove serum. The minced tissue is incubated from 1-12 hours in a freshly prepared solution of a dissociating enzyme such as trypsin. After such incubation, the dissociated cells are suspended, pelleted by centrifugation and plated onto culture dishes. All fibroblasts will attach before other cells, therefore, appropriate stromal cells can be selectively isolated and grown.

### (vii) REPROGRAMMING OF SOMATIC CELLS

The disclosure demonstrates that differentiated human cells (e.g., human dermal fibroblasts, CD34+ or mesenchymal stem cells) can be induced to de-differentiate using an ectopic mRNA expression system (e.g., an EEC system). The disclosure contemplates the use of a variety of de-differentiation coding sequences (referred to herein as RF) including, for example, a polynucleotide that encodes Klf, Oct4, Sox, Myc or SV40Tag, Lin28, Nanog or any combination thereof (e.g., Klf, Oct4, Sox, Myc or SV40Tag, Lin28, and Nanog). De-differentiation may be achieved by contacting a cell, in vivo or in vitro, with one or more EEC that remain ectopic to the host cell genome and encode at least one factor that induce de-differentiation. In various embodiments the ectopic EEC of the disclosure can be controlled by culturing a host cell transformed with the EEC transcription in the presence of B18R. Methods for promoting de-differentiation provide methods of promoting regeneration of mammalian cells and tissues damaged by injury or disease. The disclosure also provides methods for enriching for iPSC.

The generation of patient-specific iPSCs has the potential to dramatically speed the implementation of stem cells into clinical use to treat degenerative diseases. The disclosure provides methods to employ easily donated stromal cells, such as dermal fibroblasts, from a patient and generate iPSC by ectopic expression of a set of de-differentiation factors including RNA encoding (i) Klf, Oct4, Sox, MYC or SV40Tag, Nanog, Lin28 or any combination thereof; (ii) Klf, Oct4, Sox, and Lin28; and (iii) Klf, Oct4, Sox, and Nanog. The cell lines generated are physiologically and morphologically indistinguishable from Human Embryonic Stem Cells (HESC) generated from the inner cell mass of a human embryo. In particular embodiments, iPSC share a nearly identical gene expression profile with two established HESC lines. In particular embodiments, iPSC include human iPSC (hiPSC).

The terms “de-differentiation” and “reprogramming” are used interchangeably herein and are familiar to the person skilled in the relevant art. In general de-differentiation signifies the regression of lineage committed cell to the status of a pluripotent stem cell, for example, by “inducing” a de-differentiated phenotype. For example, as described further herein Klf, Oct4, Sox, Myc, SV40Tag, Lin28 and/or Nanog can induce de-differentiation and induction of mitosis in lineage committed mitotically inhibited cells.

In one embodiment, the disclosure provides a cell culture including human somatic cells that have been transfected with an EEC of the disclosure. In one embodiment the somatic cells are primary fibroblasts. In a further embodiment, the somatic cells are selected from human foreskin fibroblasts or adult fibroblasts. In another embodiment, the somatic cells are hematopoietic stem cells. In still another embodiment, the somatic cells are mesenchymal stem cells. In yet another embodiment, the cells are cultured in conditioned media including an immune evading factor (e.g., B18R) and/or are additionally transfected with a polynucleotide encoding an immune evading factor (e.g., B18R).

The disclosure also provide methods of making a stem cell (e.g., iPSC) from a somatic cell including transforming the somatic cell with an EEC transcript as described in the disclosure and culturing the somatic cell under conditions to promote expression of coding sequences in the EEC and culturing the cells for a sufficient period of time to de-differentiate the cells to stem cells (e.g., iPSC). In one embodiment, the cells are passaged at least 5, 10, 15, 20 or more times. In another embodiment, the cells are cultured for at least 10, 20, 30 or more days. In yet another embodiment, the cells are cultured in conditioned media including an immune evading factor (e.g., B18R) or are additionally transfected with a polynucleotide encoding immune evading factor (e.g., B18R).

The disclosure also provides iPSC cultures obtained by the methods described herein. In one embodiment, the iPSC do not contain any heterologous RF in the genomic DNA of the cell. In another embodiment, the iPSC do not contain any retroviral DNA or RNA (e.g., iPSC that are retroviral DNA- or RNA-free).

In particular embodiments, iPSC disclosed herein express various factors. Oligonucleotide probes and primers can be used to identify expression of various factors described herein as well as in cloning and amplification procedures. An oligonucleotide probe or a primer refers to a nucleic acid molecule of between 8 and 2000 nucleotides in length. More particularly, the length of these oligonucleotides can range from 8, 10, 15, 20, or 30 to 100 nucleotides, but will typically be 10 to 50 (e.g., 15 to 30 nucleotides). The appropriate length for oligonucleotides in assays of the disclosure under a particular set of conditions may be empirically determined by one of skill in the art.

Oligonucleotide primers and probes can be prepared by any suitable method, including, for example, cloning and restriction of appropriate sequences and direct chemical synthesis based upon the known Klf, Oct4, Sox, Myc, SV40Tag, Nanog, Lin28 or any combination thereof polynucleotide and polypeptide sequence. Various orthologs from other species are known in the art.

Oligonucleotide probes and primers can include nucleic acid analogs such as, for example, peptide nucleic acids, locked nucleic acid (LNA) analogs, and morpholino analogs. The 3′ end of the probe can be functionalized with a capture or detectable label to assist in detection of a Klf, Oct4, Sox, Myc, SV40Tag, Nanog, Lin28, or any combination thereof nucleic acid.

Any of the oligonucleotides or nucleic acid of the disclosure can be labeled by incorporating a detectable label measurable by spectroscopic, photochemical, biochemical, immunochemical, or chemical means. For example, such labels can include radioactive substances (32P, 35S, 3H, 1251), fluorescent dyes (5-bromodesoxyuridin, fluorescein, acetylaminofluorene, digoxigenin), biotin, nanoparticles, and the like. Such oligonucleotides are typically labeled at their 3′ and 5′ ends.

The oligonucleotide primers and probes can be immobilized on a solid support. Solid supports are known to those skilled in the art and include the walls of wells of a reaction tray, test tubes, polystyrene beads, magnetic beads, nitrocellulose strips, membranes, microparticles such as latex particles, glass and the like. The solid support is not critical and can be selected by one skilled in the art. Thus, latex particles, microparticles, magnetic or non-magnetic beads, membranes, plastic tubes, walls of microtiter wells, glass or silicon chips and the like are all suitable examples. Suitable methods for immobilizing oligonucleotides on a solid phase include ionic, hydrophobic, covalent interactions and the like. The solid support can be chosen for its intrinsic ability to attract and immobilize the capture reagent. The oligonucleotide probes or primers can be attached to or immobilized on a solid support individually or in groups of 2-10,000 distinct oligonucleotides of the disclosure to a single solid support. A substrate including a plurality of oligonucleotide primers or probes of the disclosure may be used either for detecting or amplifying Klf, Oct4, Sox, Myc, SV40Tag, Nanog, Lin28 or any combination thereof. For example, the oligonucleotide probes can be used in an oligonucleotide chip such as those marketed by Affymetrix and described in U.S. Pat. No. 5,143,854; PCT publications WO 90/15070 and WO 92/10092. These arrays can be produced using mechanical synthesis methods or light directed synthesis methods which incorporate a combination of photolithographic methods and solid phase oligonucleotide synthesis. The disclosure further contemplates antibodies capable of specifically binding to a Klf, Oct4, Sox, Myc, SV40Tag, Nanog, Lin28 polypeptide.

A reference or control population refers to a group of subjects or individuals who are predicted to be representative of the general population. A test sample is measured for the amount of Klf, Oct4, Sox, Myc, SV40Tag, Nanog, Lin28or any combination thereof in the sample, wherein the amount is compared to a control sample.

In another aspect, the disclosure provides methods of differentiating stem cells (e.g., iPSC) along a committed lineage including inhibiting the expression or activity of Klf, Oct4, Sox, Myc, SV40Tag, Nanog, Lin28 or any combination thereof. Differentiation agents useful in this regard include, for example, antibodies, antisense oligonucleotides, RNAi constructs, or ribozymes.

In particular embodiments, a method for reprogramming somatic cells into iPSC includes contacting isolated somatic cells with an EEC encoding a RF operably linked to a 5′ UTR or a 3′ UTR as described elsewhere herein. In particular embodiments, the method includes contacting the isolated somatic cells daily for 10 days or less, 9 days or less, 8 days or less, 7 days or less, 6 days or less, 5 days or less, or 4 days or less. In particular embodiments, the method includes contacting the isolated somatic cells daily for 4 days. In particular embodiments, the method does not include the use of feeder cells.

In particular embodiments, the method for reprogramming somatic cells into iPSC includes administering 100 ng-1000 ng of RF. In particular embodiments, one type of RF or multiple types of RF are administered. In particular embodiments, each RF is administered at a dose of 100 ng-200 ng. In particular embodiments, each RF is administered at a dose of 120 ng-150 ng. In particular embodiments, each RF is administered at a dose of 130 ng-140 ng. In particular embodiments, each RF is administered at a dose of 133.33 ng.

In particular embodiments, the method further includes administering IEFs. In particular embodiments, 150 ng-700 ng of IEFs are administered. In particular embodiments, one type or multiple types of IEFs are administered. In particular embodiments, each IEF is administered at a dose ranging from 150 ng-250 ng. In particular embodiments, each IEF is administered at a dose ranging from 180 ng-220 ng. In particular embodiments, each IEF is administered at a dose ranging from 190 ng-210 ng. In particular embodiments, each IEF is administered at a dose of 200 ng. In particular embodiments, the IEF include B18R, E3, and/or K3.

In particular embodiments, the method further includes administering microRNA. In particular embodiments, 50 ng-450 ng of microRNAs are administered. In particular embodiments, one type or multiple types of microRNAs are administered. In particular embodiments, each microRNA is administered at a dose of 50 ng-150 ng of microRNA. In particular embodiments, each microRNA is administered at a dose of 70 ng-90 ng of microRNA. In particular embodiments, each microRNA is administered at a dose of 80 ng of microRNA. In particular embodiments, each microRNA is administered at a dose of 0.1-1 μM of microRNA. In particular embodiments, each microRNA is administered at a dose of 0.4 UM of microRNA.

### (viii) IPSC ENRICHMENT

In one embodiment, the disclosure provides an enriched population of iPSC. An “enriched population of iPSC” is one wherein iPSC of the disclosure have been partially separated from other cell types, such that the resulting population of cells has a greater concentration of iPSC than the original population of cells. The enriched population of iPSC can have greater than a 10-fold, 100-fold, 500-fold, 1,000-fold, 2,000-fold, 3,000-fold, 4,000-fold, 5,000-fold, 6,000-fold, 7,000-fold, 8,000-fold, 9,000-fold, 10,000-fold or greater concentration of iPSC than the original population had prior to separation. iPSC of the disclosure can, for example, make up at least 5%, 10%, 15%, 20%, 35%, 30%, 35%, 40%, 45%, 50%, 55%, 60%, 65%, 70%, 75%, 80%, 85%, 90%, 95%, 99% or more of the enriched population of iPSC. The enriched population of iPSC may be obtained by, for example, selecting against cells displaying markers associated with differentiated cells, or other undesired cell types, and/or selecting for cells displaying markers (e.g., TRA-1-81 and/or TRA-1-60) associated with the iPSC of the disclosure, and/or by regenerating isolated iPSC in defined culture systems. Alternatively, or in addition to, the enrichment for the expression of a marker, the loss of expression of a marker may also be used for enrichment. Such enriched iPSC will lack any retroviral RNA or DNA typically used to transform cells with RF.

The iPSC of the disclosure express one or more markers associated with a pluripotent stem cell phenotype and/or lack one or more markers associated with a differentiated cell (e.g., a cell having a reduced capacity for self-renewal, regeneration, or differentiation) and/or a cell of neuronal origin. A molecule is a “marker” of a desired cell type if it is found on a sufficiently high percentage of cells of the desired cell type, and found on a sufficiently low percentage of cells of an undesired cell type. One can achieve a desired level of purification of the desired cell type from a population of cells including both desired and undesired cell types by selecting for cells in the population of cells that have the marker. A marker can be displayed on, for example, 30%, 35%, 40%, 45%, 50%, 55%, 60%, 65%, 70%, 75%, 80%, 85%, 90%, 95%, 99% or more of the desired cell type, and can be displayed on fewer than 50%, 45%, 40%, 35%, 30%, 25%, 20%, 15%, 10%, 5%, 1% or fewer of an undesired cell type.

As discussed above, the iPSC of the disclosure or iPSC that have been differentiated are characterized by the presence and/or the absence of certain markers that are specifically recognized by a molecule. Accordingly, in one aspect, the disclosure provides methods of labeling iPSC of the disclosure. In one embodiment, the iPSC are labeled with a molecule (e.g., an antibody) that specifically recognizes a marker that is associated with an iPSC of the disclosure. In another embodiment, a population of cells is contacted with a molecule that specifically binds to a marker (e.g., TRA-1-81) under conditions that allow the molecule to bind to the marker, wherein the population of cells includes at least one iPSC having said marker. In another embodiment, a population of cells is contacted with a molecule that specifically binds to a marker under conditions that allow the molecule to bind to the marker, wherein the population of cells includes iPSC that do not have the marker and non-stem cells that do have the marker. The molecule used can be, for example, an antibody, an antibody derivative, or a ligand. The molecule optionally can include an additional moiety, for example, one that is detectable (e.g., a fluorescent or colorimetric label) or one that aids in the isolation of the labeled cells (e.g., a moiety that is bound by another molecule or a magnetic particle).

In one embodiment, the population of transformed somatic cells undergoes live staining for a Tumor Rejection Antigen 1-61 and 1-81 (TRA-1-60, TRA-1-81). TRA-1-60 and TRA-1-81 may be obtained commercially, for example from Chemicon International, Inc (Temecula, Calif., USA). The immunological detection of these antigens using monoclonal antibodies has been used to characterize pluripotent stem cells in combination with other markers (Shamblott M. J. et al. (1998) PNAS 95:13726-13731; Schuldiner M. et al. (2000). PNAS 97:11307-11312; Thomson J. A. et al. (1998). Science 282:1145-1147; Reubinoff B. E. et al. (2000). Nature Biotechnology 18:399-404; Henderson J. K. et al. (2002). Stem Cells 20:329-337; Pera M. et al. (2000). J. Cell Science 113:5-10). In one embodiment, a population of somatic cells that have been reprogrammed with EEC including at least one of Klf, Oct4, Sox, or Lin28, and optionally Myc or SV40Tag are enriched for cells including TRA-1-81 or TRA-1-60 expression.

In another aspect, the disclosure provides methods of isolating iPSC of the disclosure. The iPSC of the disclosure can be isolated by, for example, utilizing molecules (e.g., antibodies, antibody derivatives, ligands or Fc-peptide fusion molecules) that bind to a marker (e.g., a TRA-1-81, a TRA-1-60 or a combination of markers) on the iPSC and thereby positively selecting cells that bind the molecule (i.e., a positive selection). Other examples of positive selection methods include methods of preferentially promoting the growth of a desired cell type in a mixed population of desired and undesired cell types. Alternatively, by using molecules that bind to markers that are not present on the desired cell type, but that are present on an undesired cell type, the undesired cells containing such markers can be removed from the desired cells (i.e., a negative selection). Other negative selection methods include preferentially killing or inhibiting the growth of an undesired cell type in a mixed population of desired and undesired cell types. Accordingly, by using negative selection, positive selection, or a combination thereof, an enriched population of iPSC can be made.

Procedures for separation may include magnetic separation, using antibody-coated magnetic beads, affinity chromatography, cytotoxic agents joined to a monoclonal antibody, or such agents used in conjunction with a monoclonal antibody, e.g., complement and cytotoxins, and “panning” with antibody attached to a solid matrix (e.g., plate), or other convenient technique. Techniques providing accurate separation include fluorescence activated cell sorters, which can have varying degrees of sophistication, e.g., a plurality of color channels, low angle and obtuse light scattering detecting channels, and impedance channels. Conveniently, antibodies may be conjugated with markers, such as magnetic beads, which allow for direct separation, biotin, which can be removed with avidin or streptavidin bound to a support, fluorochromes, which can be used with a fluorescence activated cell sorter, or the like, to allow for ease of separation of the particular cell type. Any technique may be employed which is not unduly detrimental to the viability of the iPSC. In one embodiment, the cells are incubated with an antibody against a marker (e.g., a TRA-1-81 antibody) and the cells that stain positive for the marker are manually selected and subcultured. In particular embodiments, iPSCs are isolated by bulk passaging cells three to five times. During passaging, cells are allowed to attach to the cell culture vessel. At each passage, cells that are not attached are removed; attached cells are then detached and passaged. Passaged cells are allowed to reattach to the cell culture vessel.

Combinations of enrichment methods may be used to improve the time or efficiency of purification or enrichment. For example, after an enrichment step to remove cells having markers that are not indicative of the cell type of interest the cells may be further separated or enriched by a fluorescence activated cell sorter (FACS) or other methodology having high specificity. Multi-color analyses may be employed with a FACS. The cells may be separated on the basis of the level of staining for a particular antigen or lack thereof. Fluorochromes may be used to label antibodies specific for a particular antigen. Such fluorochromes include phycobiliproteins, e.g., phycoerythrin and allophycocyanins, fluorescein, Texas red, and the like. In particular embodiments, iPSCs are enriched by FACS.

Any cell type-specific markers can be used to select for or against a particular cell type. iPSC markers useful for enrichment include expressed markers such as TRA-1-81 and loss of markers (e.g., GFP) associated with a retroviral vector or other exogenous vector.

### (ix) Culture and Storage of Cells

In another embodiment, the disclosure provides methods of establishing and/or maintaining populations of stem cells (e.g., iPSC), or the progeny thereof, as well as mixed populations including both stem cells (e.g., iPSC) and progeny cells, and the populations of cells so produced. As with the iPSC, once a culture of cells or a mixed culture of stem cells (e.g., iPSC) is established, the population of cells is mitotically expanded in vitro by passage to fresh medium as cell density dictates under conditions conducive to cell proliferation, with or without tissue formation. Such culturing methods can include, for example, passaging the cells in culture medium lacking particular growth factors that induce differentiation (e.g., IGF, EGF, FGF, VEGF, and/or other growth factor), in the presence of an agent that stimulates (e.g., an agonist) of Klf, Oct4, Sox, Myc, SV40Tag, Nanog, Lin28 or any combination thereof, in the presence of Klf, Oct4, Sox, Myc, SV40Tag, Nanog, Lin28 or any combination thereof, or any combination of the foregoing. Mixed cultures including the original somatic cells (e.g., fibroblast, hematopeitic stem cells, mesenchymal stem cells, or fibroblast-like cells) and reprogrammed stem cells (e.g., iPSC) can be transferred to fresh medium when sufficient cell density is reached. Some stem cell types do not demonstrate typical contact inhibition-apoptosis or they become quiescent when density is maximum. Accordingly, appropriate passaging techniques can be used to reduce contact inhibition and quiescence. Thus, in one embodiment, for example, transferring a portion of the cells to a new culture vessel with fresh medium. Such removal or transfer can be done in any culture vessel.

In particular embodiments, the disclosure provides cell lines of iPSC. As used herein a “cell line” means a culture of stem cells (e.g., iPSC) of the disclosure, or progeny cells thereof, that can be reproduced for an extended period of time, preferably indefinitely, and which term includes, for example, cells that are cultured, cryopreserved and re-cultured following cryopreservation. As used herein a “culture” means a population of iPSC grown in a medium and optionally passaged accordingly. A stem cell (e.g., iPSC) culture may be a primary culture (e.g., a culture that has not been passaged) or may be a secondary or subsequent culture (e.g., a population of cells which have been subcultured or passaged one or more times).

Once the iPSC have been established in culture, as described above, they may be maintained or stored in cell “banks” including either continuous in vitro cultures of cells requiring regular transfer or cells which have been cryopreserved. In some embodiments, the banked cells are used for autologous treatment of a subject.

Cryopreservation of stem cells (e.g., iPSC), or other cell of the disclosure, may be carried out according to known methods, such as those described in Doyle et al., (eds.), 1995, Cell & Tissue Culture: Laboratory Procedures, John Wiley & Sons, Chichester. For example, cells may be suspended in a “freeze medium” such as, for example, culture medium further including 15-20% fetal bovine serum (FBS) and 10% dimethylsulfoxide (DMSO), with or without 5-10% glycerol, at a density, for example, of 4-10×10⁶ cells/ml. The cells are dispensed into glass or plastic vials which are then sealed and transferred to a freezing chamber of a programmable or passive freezer. The optimal rate of freezing may be determined empirically. For example, a freezing program that gives a change in temperature of −1° C./min through the heat of fusion may be used. Once vials containing the cells have reached −80° C., they are transferred to a liquid nitrogen storage area. Cryopreserved cells can be stored for a period of years, though they should be checked at least every 5 years for maintenance of viability.

The cryopreserved cells of the disclosure constitute a bank of cells, portions of which can be withdrawn by thawing and then used to produce a stem cell (e.g., iPSC) culture including stem cells, as needed. Thawing should generally be carried out rapidly, for example, by transferring a vial from liquid nitrogen to a 37° C. water bath. The thawed contents of the vial should be immediately transferred under sterile conditions to a culture vessel containing an appropriate medium. It is advisable that the cells in the culture medium be adjusted to an initial density of 1-3×10⁵ cells/ml. Once in culture, the cells may be examined daily, for example, with an inverted microscope to detect cell proliferation, and subcultured as soon as they reach an appropriate density.

The iPSC of the disclosure may be withdrawn from a cell bank as needed, and used for the production of new stem cells, either in vitro, for example, as a three dimensional tissue culture, as described below, or in vivo, for example, by direct administration of cells to the site where new fibroblasts or tissue is needed. As described herein, the iPSC of the disclosure may be used to produce new tissue for use in a subject where the cells were originally isolated from that subject's own blood or other tissue (i.e., autologous cells). Alternatively, the cells of the disclosure may be used as ubiquitous donor cells to produce new tissue for use in any subject (i.e., heterologous cells).

Once established, a culture of stem cells (e.g., iPSC) may be used to produce progeny cells and/or fibroblasts capable of producing new tissue. Differentiation of stem cells (e.g., iPSC) to fibroblasts or other cell types, followed by the production of tissue therefrom, can be triggered by specific exogenous growth factors or by changing the culture conditions (e.g., the density) of a stem cell (e.g., iPSC) culture. Since the cells are pluripotent, they can be used to reconstitute an irradiated subject and/or a subject treated with chemotherapy; or as a source of cells for specific lineages, by providing for their maturation, proliferation and differentiation into one or more selected lineages. Examples of factors that can be used to induce differentiation include erythropoietin, colony stimulating factors, e.g., GM-CSF, G-CSF, or M-CSF, interleukins, e.g., IL-1, -2, -3, -4, -5, -6, -7, -8, and the like, Leukemia Inhibitory Factory (LIF), Steel Factor (Stl), or the like, coculture with tissue committed cells, or other lineage committed cells types to induce the stem cells (e.g., iPSC) into becoming committed to a particular lineage. Additional methods of differentiation are described in more detail elsewhere herein.

(x) Formulations and Three-Dimensional Constructs. In particular embodiments, iPSC can be harvested from a culture medium and washed and concentrated into a carrier in a therapeutically-effective amount. Exemplary carriers include saline, buffered saline, physiological saline, water, Hanks' solution, Ringer's solution, Normosol-R (Abbott Labs), PLASMA-LYTE A® (Baxter Laboratories, Inc., Morton Grove, IL), and combinations thereof.

In particular embodiments, carriers can be supplemented with human serum albumin (HSA) or other human serum components or fetal bovine serum. In particular embodiments, a carrier for infusion includes buffered saline with 5% HSA or dextrose. Additional isotonic agents include polyhydric sugar alcohols including trihydric or higher sugar alcohols, such as glycerin, erythritol, arabitol, xylitol, sorbitol, or mannitol.

Carriers can include buffering agents, such as citrate buffers, succinate buffers, tartrate buffers, fumarate buffers, gluconate buffers, oxalate buffers, lactate buffers, acetate buffers, phosphate buffers, histidine buffers, and/or trimethylamine salts.

Stabilizers refer to a broad category of excipients which can range in function from a bulking agent to an additive which helps to prevent cell adherence to container walls. Typical stabilizers can include polyhydric sugar alcohols; amino acids, such as arginine, lysine, glycine, glutamine, asparagine, histidine, alanine, ornithine, L-leucine, 2-phenylalanine, glutamic acid, and threonine; organic sugars or sugar alcohols, such as lactose, trehalose, stachyose, mannitol, sorbitol, xylitol, ribitol, myoinisitol, galactitol, glycerol, and cyclitols, such as inositol; PEG; amino acid polymers; sulfur-containing reducing agents, such as urea, glutathione, thioctic acid, sodium thioglycolate, thioglycerol, alpha-monothioglycerol, and sodium thiosulfate; low molecular weight polypeptides (i.e., <10 residues); proteins such as HSA, bovine serum albumin, gelatin or immunoglobulins; hydrophilic polymers such as polyvinylpyrrolidone; monosaccharides such as xylose, mannose, fructose and glucose; disaccharides such as lactose, maltose and sucrose; trisaccharides such as raffinose, and polysaccharides such as dextran.

Where necessary or beneficial, formulations can include a local anesthetic such as lidocaine to ease pain at a site of injection.

Exemplary preservatives include phenol, benzyl alcohol, meta-cresol, methyl paraben, propyl paraben, octadecyldimethylbenzyl ammonium chloride, benzalkonium halides, hexamethonium chloride, alkyl parabens such as methyl or propyl paraben, catechol, resorcinol, cyclohexanol, and 3-pentanol.

Therapeutically effective amounts of cells within formulations can be greater than 10² cells, greater than 10³ cells, greater than 10⁴ cells, greater than 10⁵ cells, greater than 10⁶ cells, greater than 10⁷ cells, greater than 10⁸ cells, greater than 10⁹ cells, greater than 10¹⁰ cells, or greater than 10¹¹.

In formulations disclosed herein, cells are generally in a volume of a liter or less, 500 ml or less, 250 ml or less or 100 ml or less. Hence the density of administered cells is typically greater than 10⁴ cells/ml, 10⁷ cells/ml or 10⁸ cells/ml.

The cell-based formulations disclosed herein can be prepared for administration by, e.g., injection, infusion, perfusion, or lavage. The formulations can further be formulated for bone marrow, intravenous, intradermal, intraarterial, intranodal, intralymphatic, intraperitoneal, intralesional, intraprostatic, intravaginal, intrarectal, intrathecal, intratumoral, intramuscular, intravesicular, and/or subcutaneous injection.

(xi) Differentiation of iPSC. In particular embodiments, iPSC are differentiated, for example, for a research or therapeutic purpose (e.g., before administration to a subject). Where differentiation of iPSC is desired, stem cells (e.g., iPSC) can be exposed to one or more activation factors (e.g., growth factors, differentiation factors, and/or survival factors) that promote differentiation into a more committed cell type.

Many activation factors and cell culture conditions that promote differentiation are known in the art (see, e.g., U.S. Pat. No. 7,399,633 at Section 5.2 and Section 5.5). For example, stem cell factor (SCF) can be used in combination with granulocyte-macrophage colony-stimulating factor (GM-CSF) or interleukin (IL)-7 to promote differentiation into myeloid stem/progenitor cells or lymphoid stem/progenitor cells, respectively. In particular embodiments, iPSC can be differentiated into a lymphoid stem/progenitor cell by exposing iPSC to 100 ng/ml of each of SCF and GM-CSF or IL-7. In particular embodiments, a retinoic acid receptor (RAR) agonist, or preferably all trans retinoic acid (ATRA) is used to promote the differentiation of iPSC. Differentiation into natural killer cells, e.g., can be achieved by exposing cultured iPSC to RPMI media supplemented with human serum, IL-2 at 50 U/mL and IL-15 at 500 ng/ml. In additional embodiments, RPMI media can also be supplemented L-glutamine.

Cardiomyocytes have been generated in vitro from a wide range of stem cells, including iPSC (see, e.g., Gai, et al., 2009, Cell. Biol. Int. 33:1184-93; Kuzmenkin, et al., 2009, FASEB J. 23:4168-80; Pfannkuchent al., 2009, Cell Physiol. Biochem. 24:73-86), ESCs (see, e.g., Beqqali, et al., 2009, Cell. Mol. Life Sci. 66:800-13; Steel, et al., 2009, Curr. Opin. Drug Discov. Dev 12:133-40), HSPC (see, e.g., Choi, et al., 2008, Biotechnol. Lett 30:835-43; Antonitsis, et al., 2008, Thorac. Cardiovasc. Surg 56:77-82; Ge, et al., 2009, Biochem. Biophys. Res. Commun. 381:317-21; Gwak, et al., 2009, Cell. Biochem. Funct. 27:148-54), and cardiomyocyte progenitor cells (see, e.g., Smits, et al., 2009, Nat. Protoc. 4:232-43). Mummery, et al., 2012 Jul. 20, Circ. Res. 111 (3): 344-358 provides a summary of methods to differentiate iPSC and hESCs into cardiomyocytes. Methods to differentiate stem cells (e.g., iPSC) into cardiac cells are also described in, e.g., U.S. Publication No. 2015/0017718.

In particular embodiments, cardiomyocyte progenitors can be generated from embryoid bodies (EBs) treated with Activin A, BMP4 or with 2+Wnt3 and bFGF. These progenitors express Nkx2.5, Tbx5/20, Gata-4, Mef2c and Hand1/2. Their further differentiation to functional cardiomyocytes can be promoted with VEGF and Dkk1 (Vidarsson, et al., 2010, Stem Cell Rev. 6:108-20).

A protocol for generating insulin producing beta-cells involves stepwise lineage restriction generating in sequence: definitive endodermal cells (Activin+Wnt3), primitive foregut endoderm (FGF10+KAAD-cyclopamine), posterior foregut endoderm (RA+FGF10+KAAD-cyclopamine), pancreatic endoderm and endocrine precursors (Extendin-4), and hormone producing cells (IGF1+HGF). Transcription factor profiles include: Sox17, CER, FoxA2, and the cytokine receptor CXCR4 (definitive endodermal cells), Hnf1B, Hnf4A (primitive foregut endoderm), Pdx1, Hnf6, H1xB9 (posterior foregut endoderm), and Nkx6.1, Nkx2.2, Ngn3, Pax4 (pancreatic endoderm and endocrine precursors). See, e.g., D'Amour, et al., 2006, Nat. Biotechnol. 24:1392-401; Kroon, et al., 2008, Nat. Biotechnol. 26:443-52). Another method to induce stem cells (e.g., iPSC) to commit to definitive endoderm, then to pancreatic endoderm, to pancreatic endocrine/exocrine cells and finally to more mature islet cells is described in Jiang, et al., 2007, Stem Cells 25 (8): p. 1940-53.

Various types of retinal cells can be generated from stem cells (e.g., iPSC) (see, e.g., Lamba, et al., 2006, Proc. Natl. Acad. Sci. USA 103:12769-74; Reh, et al., 2010, Methods Mol. Biol. 636:139-53). EBs can be produced and thereafter treated with IGF1, Noggin (BMP inhibitor) and Dkk1 (Wnt inhibitor). This treatment with IGF1, Noggin (BMP inhibitor), and Dkk1 (Wnt inhibitor) can direct stem cells (e.g., iPSC) to adopt a retinal progenitor phenotype, expressing Pax6 and Chx10. Exposing these progenitors to N—(N-(3,5-difluorophenacetyl)-1-alanyl)-S-phenylglycine t-butyl ester (DAPT), a blocker of Notch signaling, promotes neuronal differentiation (Lamba, et al., 2010, PLOS One 5: e8763). The decision to undergo photoreceptor differentiation is under the control of the transcription factor, Blimp1 (Brzezinski, et al., 2010, Development 137:619-29).

In particular embodiments, neuronal differentiation can be achieved by replacing a stem cell culture media with a media including basic fibroblast growth factor (bFGF) heparin, and an N2 supplement (e.g., transferrin, insulin, progesterone, putrescine, and selenite). Two days later, differentiating cells can be attached by plating them onto dishes coated with laminin or polyornithine. After an additional 10-11 days in culture, primitive neuroepithelial cells will have formed. The identity of the cells can be confirmed by staining for PAX6 (paired box protein 6, a transcription factor), SOX2 (sex-determining region Y-box 2, another transcription factor), and N-cadherin (a calcium-dependent cell adhesion molecule specific to neural tissue). Neuroepithelial cells can be further differentiated into, e.g., motor neurons (see, e.g., Li, et al. 2005, Nat. Biotechnol. 23, 215-221), dopaminergic neurons (see, e.g., Yan, et al. 2005, Stem Cells 23, 781-790), and oligodendrocytes (Nistor, et al. 2005, Glia 49, 385-396).

Additional information regarding differentiation to motor neurons includes treatment with RA (Pax6 expressing primitive neuroepithelial cells), RA+Shh (Pax6/Sox1 expressing neuroepithelial cells), which gradually start to express the motor neuron progenitor marker Olig2. Reducing RA+Shh concentration promotes the emergence of motor neurons expressing HB9 and Islet1. The addition of brain-derived neurotrophic factor (BDNF), glial-derived neurotrophic factor (GDNF), insulin-like growth factor-1 (IGF1), and cAMP promotes process outgrowth (see, e.g., Hu, et al., 2009, Nat. Protoc. 4:1614-22; Hu, et al., 2010, Proc. Natl. Acad. Sci. USA; 107:4335-40).

Additional information regarding differentiation to dopaminergic neurons includes overexpression of the transcription factor Nurr1 followed by exposure to Shh, FGF-8 and ascorbic acid (see, e.g., Lee, et al., 2000 June, Nat. Biotechnol. 18 (6): 675-9; Kriks and Studer, 2009, Adv. Exp. Med. Biol. 651:101-11; Lindvall and Kokaia, 2009 May, Trends Pharmacol. Sci. 30 (5): 260-7). The combination of stromal cell-derived factor 1 (SDF-1/CXCL12), pleiotrophin (PTN), insulin-like growth factor 2 (IGF2), and ephrin B1 (EFNB1) can induce stem cells (e.g., iPSC) to differentiate to TH-positive neurons in vitro, expressing midbrain specific markers, including Engrailed 1, Nurr1, Pitx3, and dopamine transporter (DAT). These neurons are capable of generating action potentials and forming functional synaptic connections (Vazin, et al., 2009, PLOS One 4: e6606).

A protocol to produce mature myelinating oligodendrocytes includes directing stem cells (e.g., iPSC) toward neuroectoderm differentiation in the absence of growth factors for 2 weeks. These cells express neuroectoderm transcription factors, including Pax6 and Sox1. Next stem cells (e.g., iPSC) are exposed to the caudalizing factor retinoic acid (RA) and the ventralizing morphogen Shh for 10 days to begin expression of Olig2. To prevent the differentiation to motor neurons and promote the generation of oligodendrocyte precursor cells (OPC) s, cells are cultured with FGF2 for 10 days. By day 35, the Olig2 progenitors co-express NkxX2.2 and no longer give rise to motor neurons. The co-expression of Olig2 and Nkx2.2 reflects a stage prior to human OPCs (pre-OPCs). These pre-OPCs are finally cultured in a glia medium including triiodothyronine (T3), neurotrophin 3 (NT3), PDGF, CAMP, IGF-1 and biotin, which individually or synergistically can promote the survival and proliferation of the OPCs, for another 8 weeks to generate OPCs. These OPCs are bipolar or multipolar, express Olig2, Nkx2.2, Sox10 and PDGFRα, become motile and are able to differentiate to competent oligodendrocytes. WO2007/066338 also describes differentiation protocols for the generation of oligodendrocyte-like cells.

A protocol to produce glutamatergic neurons includes use of stem cells (e.g., iPSC) to produce cell aggregates which are then treated for 8 days with RA. This results in Pax6 expressing radial glial cells, which after additional culturing in N2 followed by “complete” medium results in 95% glutamate neurons (Bibel, et al., 2007, Nat. Protoc. 2:1034-43).

A protocol to produce GABAergic neurons includes exposing EBs for 3 days to all-trans-RA. After subsequent culture in serum-free neuronal induction medium including Neurobasal medium supplemented with B27, bFGF and EGF, 95% GABA neurons develop (see, e.g., Chatzi, et al., 2009, Exp. Neurol. 217:407-16).

U.S. Publication No. 2013/0330306 describes compositions and methods to induce differentiation and proliferation of neural precursor cells or neural stem cells into neural cells using umbilical cord blood-derived mesenchymal stem cells; U.S. Publication No. 2007/0179092 describes use of pituitary adenylate cyclase activating polypeptide (PACAP) to enhance neural stem cell proliferation, differentiation and survival; U.S. Publication No. 2012/0329714 describes use of prolactin to increase neural stem cell numbers; while U.S. Publication No. 2012/0308530 describes a culture surface with amino groups that promotes neuronal differentiation into neurons, astrocytes and oligodendrocytes. U.S. Publication No. 2006/211109 describes improved methods for efficiently producing neuroprogenitor cells and differentiated neural cells such as dopaminergic neurons and serotonergic neurons from pluripotent stem cells, e.g., iPSCs.

Thus, the fate of neural stem cells can be controlled by a variety of extracellular factors. Commonly used factors include amphiregulin; BMP-2 (U.S. Pat. Nos. 5,948,428 and 6,001,654); brain derived growth factor (BDNF; Shetty and Turner, 1998, J. Neurobiol. 35:395-425); neurotrophins (e.g., Neurotrophin-3 (NT-3) and Neurotrophin-4 (NT-4); Caldwell, et al., 2001, Nat. Biotechnol. 1; 19:475-9); ciliary neurotrophic factor (CNTF); cyclic adenosine monophosphate; epidermal growth factor (EGF); dexamethasone (glucocorticoid hormone); fibroblast growth factor (bFGF; U.S. Pat. No. 5,766,948; FGF-1, FGF-2); forskolin; GDNF family receptor ligands; growth hormone; interleukins; insulin-like growth factors; isobutyl 3-methylxanthine; leukemia inhibitory growth factor (LIF; U.S. Pat. No. 6,103,530); Notch antagonists (U.S. Pat. No. 6,149,902); platelet derived growth factor (PDGF; U.S. Pat. No. 5,753,506); potassium; retinoic acid (U.S. Pat. No. 6,395,546); somatostatin; tetanus toxin; and transforming growth factor-α and TGF-β (U.S. Pat. Nos. 5,851,832 and 5,753,506).

In particular embodiments, preferred proliferation-inducing neural growth factors include BNDF, EGF and FGF-1 or FGF-2. Growth factors can be usually added to the culture medium at concentrations ranging between 1 fg/ml of a pharmaceutically acceptable composition (including, e.g., CNS compatible carriers, excipients and/or buffers) to 1 mg/ml.

Growth factor expanded stem cells (e.g., iPSC) can also differentiate into neurons and glia after mitogen withdrawal from a culture medium.

Additionally, WO 2004/046348 describes differentiation protocols for the generation of neural-like cells from bone marrow-derived stem cells. WO 2006/134602 describes differentiation protocols for the generation of neurotrophic factor secreting cells. Commercial kits are also available from Life Technologies and include PSC Neural Induction Medium, Geltrex™ LDEV-Free hESC-qualified Reduced Growth Factor Basement Membrane Matrix, and a Human Neural Stem Cell Immunocytochemistry kit. Stem cells (e.g., iPSC) differentiated into neural cells using the Life Technology kits can be further terminally differentiated into neurons, astrocytes and oligodendrocytes using Life Technologies' B-27® supplements, with N−2 supplement and NEUROBASAL® Medium.

Additional methods to assist with stem cell (e.g., iPSC) differentiation protocols include, e.g., culture vessels with a portion including an oxygen permeable substrate at least partially coated with a synthetic matrix having an average thickness of less than 100 nm. See, e.g., U.S. Publication No. 2014/0370598.

U.S. Publication No. 2013/0251690 describes methods to support stem cell (e.g., iPSC) differentiation in elderly populations.

A number of different differentiation methods have been described. Additional methods that can be used within the teaching of the current disclosure can be found in the art by those with ordinary skill. Furthermore, and as indicated, differentiation of stem cells (e.g., iPSC) can be confirmed by measuring cellular markers expressed by the desired differentiated cell.

The foregoing discussion describes in vitro or ex vivo differentiation methods. Modified stem cells (e.g., iPSC) disclosed herein can also differentiate in vivo following administration, as described elsewhere herein.

(xii) Methods of Use. Methods disclosed herein include treating subjects (humans, non-human primates, veterinary animals (dogs, cats, reptiles, birds, etc.) livestock (horses, cattle, goats, pigs, chickens, etc.) and research animals (monkeys, rats, mice, fish, etc.)) with formulations disclosed herein. Treating subjects includes delivering therapeutically effective amounts. Therapeutically effective amounts include those that provide effective amounts, prophylactic treatments and/or therapeutic treatments.

An “effective amount” is the amount of a formulation necessary to result in a desired physiological change in the subject. For example, an effective amount can provide an anti-cancer, anti-infection, anti-diabetic, or healing effect. Effective amounts are often administered for research purposes. Effective amounts disclosed herein can cause a statistically significant effect in an animal model or in vitro assay relevant to the assessment of a disease, disorder, or injury's development or progression.

A “prophylactic treatment” includes a treatment administered to a subject who does not display signs or symptoms of a disease, disorder, or injury or displays only early signs or symptoms of a disease, disorder, or injury such that treatment is administered for the purpose of diminishing or decreasing the risk of developing the disease, disorder, or injury further. Thus, a prophylactic treatment functions as a preventative treatment against a disease, disorder, or injury. In particular embodiments, prophylactic treatments reduce, delay, or prevent disease, disorder, or injury.

A “therapeutic treatment” includes a treatment administered to a subject who displays symptoms or signs of a disease, disorder, or injury and is administered to the subject for the purpose of diminishing or eliminating those signs or symptoms of the disease, disorder, or injury. The therapeutic treatment can reduce, control, or eliminate the presence or activity of the disease, disorder, or injury and/or reduce control or eliminate side effects of the disease, disorder, or injury.

Function as an effective amount, prophylactic treatment or therapeutic treatment are not mutually exclusive, and in particular embodiments, administered dosages may accomplish more than one treatment type.

Uses of the iPSC include transplanting the iPSC, stem cell populations, or progeny thereof into subjects to treat a variety of pathological states including diseases and disorders resulting from cancers, neoplasms, injury, viral infections, diabetes and the like. Stem cells or stem cell populations (including genetically altered stem cells) are introduced into a subject in need of such stem cells or progeny or in need of a molecule encoded or produced by the genetically altered cell.

The iPSC, their progeny, and tissue of the disclosure can be used in a variety of applications. These include: transplantation or implantation of the cells either in a differentiated form, an undifferentiated form, a de-differentiated form. Such cells and tissues serve to repair, replace or augment tissue that has been damaged due to disease or trauma, or that failed to develop normally.

In one embodiment, a formulation including the cells of the disclosure is prepared for injection directly to the site where the production of new tissue is desired. For example, the cells of the disclosure may be suspended in a hydrogel solution for injection. Alternatively, the hydrogel solution containing the cells may be allowed to harden, for instance in a mold to form a matrix having cells dispersed therein prior to implantation. Once the matrix has hardened, the cell formations may be cultured so that the cells are mitotically expanded prior to implantation. A hydrogel is an organic polymer (natural or synthetic) which is cross-linked via covalent, ionic, or hydrogen bonds to create a three-dimensional open-lattice structure, which entraps water molecules to form a gel. Examples of materials which can be used to form a hydrogel include polysaccharides such as alginate and salts thereof, polyphosphazines, and polyacrylates, which are cross-linked ionically, polyethylene oxide-polypropylene glycol block copolymers which are cross-linked by temperature or pH, respectively. Methods of synthesis of the hydrogel materials, as well as methods for preparing such hydrogels, are known in the art.

Such cell formulations may further include one or more other components, including selected extracellular matrix components, such as one or more types of collagen known in the art, and/or growth factors and drugs. Growth factors which may be usefully incorporated into the cell formulation include one or more tissue growth factors known in the art such as: any member of the transforming growth factor (TGF)-β family, insulin-like growth factor (IGF)-1 and -2, growth hormone, bone morphogenetic proteins (BMPs) such as BMP-13, and the like. Alternatively, the cells of the disclosure may be genetically engineered to express and produce growth factors such as BMP-13 or TGF-β. Other components may also be included in the formulation include, for example, buffers to provide appropriate pH and isotonicity, lubricants, viscous materials to retain the cells at or near the site of administration, (e.g., alginates, agars and plant gums) and other cell types that may produce a desired effect at the site of administration (e.g., enhancement or modification of the formation of tissue or its physicochemical characteristics, support for the viability of the cells, or inhibition of inflammation or rejection). The cells can be covered by an appropriate wound covering to prevent cells from leaving the site. Such wound coverings are known to those of skill in the art.

Alternatively, the iPSC of the disclosure may be seeded onto a three-dimensional framework or scaffold and cultured to allow the cells to differentiate, grow and fill the matrix or immediately implanted in vivo, where the seeded cells will proliferate on the surface of the framework and form a replacement tissue in vivo in cooperation with the cells of the subject. Such a framework can be implanted in combination with any one or more growth factors, drugs, additional cell types, or other components that stimulate formation or otherwise enhance or improve the practice of the disclosure.

The cells may be introduced directly into the peripheral blood or deposited within other locations throughout the body, e.g., a desired tissue, or on microcarrier beads in the peritoneum.

The cells of the disclosure may be used to treat subjects requiring the repair or replacement of tissue resulting from disease or trauma. Treatment may entail the use of the cells of the disclosure to produce new tissue, and the use of the tissue thus produced, according to any method presently known in the art or to be developed in the future. For example, the induced cells (e.g., cells including an ectopic expression vector expressing Klf, Oct4, Sox, Myc, SV40Tag, Nanog, Lin28 or any combination thereof) of the disclosure may be implanted, injected or otherwise administered directly to the site of tissue damage so that they will produce new tissue in vivo. In one embodiment, administration includes the administration of genetically modified stem cells (e.g., iPSC).

In yet another embodiment, the iPSC of the disclosure can be used in conjunction with a three-dimensional culture system in a “bioreactor” to produce tissue constructs which possess critical biochemical, physical and structural properties of native human tissue by culturing the cells and resulting tissue under environmental conditions which are typically experienced by native tissue. The bioreactor may include a number of designs. Typically, the culture conditions will include placing a physiological stress on the construct containing cells similar to what will be encountered in vivo.

For example, in one embodiment, the iPSC can be administered to cancer patients who have undergone chemotherapy that have killed, reduced, or damaged stem cells or other cells of a subject, wherein the iPSC replace the damaged or dead cells. In another embodiment, the iPSC can be transfected or transformed (in addition to the de-differentiation factors) with at least one additional therapeutic factor. For example, once iPSC of the disclosure are isolated or obtained by the methods of the disclosure, the iPSC may be transformed with a polynucleotide encoding a therapeutic polypeptide. Method and compositions can provide stem cell bioreactors for the production of a desired polypeptide or may be used for gene delivery or gene therapy. In this embodiment, the iPSC may be isolated, transformed with a polynucleotide encoding a therapeutic polypeptide and may then be implanted or administered to a subject, or may be differentiated to a desired cell type and implanted and delivered to the subject. Under such conditions the polynucleotide is expressed within the subject for delivery of the polypeptide product.

Stem cells (e.g., iPSC) which express a gene product of interest, or tissue produced in vitro therefrom, can be implanted into a subject who is otherwise deficient in that gene product. For example, genes that express products capable of preventing or ameliorating symptoms of various types of vascular diseases or disorders, or that prevent or promote inflammatory disorders are of particular interest. In one embodiment, the cells of the disclosure are genetically engineered to express an anti-inflammatory gene product that would serve to reduce the risk of failure of implantation or further degenerative change in tissue due to inflammatory reaction. For example, a iPSC of the disclosure can be genetically engineered to express one or more anti-inflammatory gene products including, for example, peptides or polypeptides corresponding to the idiotype of antibodies that neutralize granulocyte-macrophage colony stimulating factor (GM-CSF), tumor necrosis factor (TNF), IL-1, IL-2, or other inflammatory cytokines. IL-1 has been shown to decrease the synthesis of proteoglycans and collagens type II, IX, and XI (Tyler et al., 1985, Biochem. J. 227:69-878; Tyler et al., 1988, Coll. Relat. Res. 82:393-405; Goldring et al., 1988, J. Clin. Invest. 82:2026-2037; and Lefebvre et al., 1990, Biophys. Acta. 1052:366-72). TNF also inhibits synthesis of proteoglycans and type II collagen, although it is much less potent than IL-1 (Yaron, I., et al., 1989, Arthritis Rheum. 32:173-80; Ikebe, T., et al., 1988, J. Immunol. 140:827-31; and Saklatvala, J., 1986, Nature 322:547-49). Also, for example, the cells of the disclosure may be engineered to express the gene encoding the human complement regulatory protein that prevents rejection of a graft by the host. See, for example, McCurry et al., 1995, Nature Medicine 1:423-27. In another embodiment, the iPSC may be engineered to include a gene or polynucleotides sequence that expresses or causes to be expressed an angiogenic factor.

It has been previously demonstrated that transplantation of beta islet cells provides therapy for patients with diabetes (Shapiro et al., N. Engl. J. Med. 343:230-238, 2000). The iPSC provide an alternative source of islet cells to prevent or treat diabetes. For example, iPSC of the disclosure can be generated, isolated and differentiated to a pancreatic cell type and delivered to a subject. Alternatively, the iPSC can be delivered to the pancreas of the subject and differentiated to islet cells in vivo. Accordingly, the cells are useful for transplantation in order to prevent or treat the occurrence of diabetes.

In another embodiment, the iPSC are genetically engineered to express genes for specific types of growth factors for successful and/or improved differentiation to fibroblasts, other stromal cells, or parenchymal cells and/or turnover either pre- or post-implantation.

Differentiation of the iPSC or de-differentiation of lineage committed (mitotically inhibited) cells can be induced ex vivo, or alternatively may be induced by contact with tissue in vivo, (e.g., by contact with fibroblasts or cell matrix components). Optionally, a differentiating agent or de-differentiation agent (e.g., Klf, Oct4, Sox, Myc, SV40Tag, Nanog, Lin28, or any combination thereof or an agonist thereof) may be co-administered or subsequently administered to the subject.

The disclosure contemplates that the in vitro methods described herein can be used for autologous transplantation of de-differentiated or redifferentiated cells (e.g., the cells are harvested from and returned to the same individual). The disclosure further contemplates that the in vitro methods described herein can be used for non-autologous transplantations. In one embodiment, the transplantation occurs between a genetically related donor and recipient. In another embodiment, the transplantation occurs between a genetically un-related donor and recipient. In any of the foregoing embodiments, the disclosure contemplates that de-differentiated cells can be expanded in culture and stored for later retrieval and use. Similarly, the disclosure contemplates that redifferentiated cells can be expanded in culture and stored for later retrieval and use. In particular embodiments, where the de-differentiated cells are to be used for transplantation or implantation in vivo it is useful to obtain the somatic cells from the patient's own tissues.

The compositions and methods of the disclosure may be applied to a procedure wherein differentiated (lineage committed) cells are removed from a subject, de-differentiated in culture, and then either reintroduced into that individual or, while still in culture, manipulated to redifferentiate along specific differentiation pathways (e.g., pancreatic cells, neuronal cells, liver cells, skin cells, cardiovascular cells, gastrointestinal cells and the like). Such redifferentiated cells can then be introduced to the individual. For example, differentiated fibroblasts can be removed, de-differentiated (e.g., with ectopic expression of a EEC of the disclosure including Klf, Oct4, Sox, Myc, SV40Tag, Nanog, Lin28 or any combination thereof) and mitotically expanded and then re-differentiated (e.g., with a Klf, Oct4, Sox, Myc, SV40Tag, Nanog, Lin28, antagonists or any combination thereof) or factors (including physical stimuli) known to cause differentiation of hESCs down a lineage committed path. In one embodiment, the method includes removing differentiated cells from an injured or diseased subject. Cells de-differentiated from cells harvested from an injured subject can later be returned to the injured or diseased subject to treat an injury or degenerative disease. The de-differentiated cells can be reintroduced at the site or injury, or the cells can be reintroduced at a site distant from the injury. Similarly, cells can be harvested from an injured subject, de-differentiated in vitro, redifferentiated in vitro, and transplanted back to the subject to treat an injury or degenerative disease.

In particular embodiments, methods can include heterologous administration of iPSC disclosed herein with the iPSC differentiating following administration. In particular embodiments, components that support activation (e.g., expansion, differentiation and/or survival) of iPSC in vitro can be administered in combination with iPSC to direct differentiation and survival following administration in vivo. In general, activation factors include any proteins, peptides or other molecules having a growth, proliferative, differentiative, or trophic effect on iPSC and/or iPSC progeny. Activation factors which may be used for inducing proliferation include any trophic factor that allows stem cells (e.g., iPSC) and precursor cells to proliferate, including any molecule which binds to a receptor on the surface of the cell to exert a trophic, or growth-inducing effect on the cell.

To support differentiation and/or survival of iPSC in vivo, stem-cell activation factors may be delivered or formulated for timed-release. Several examples of time-release formulations that may be used are described in, e.g., WO 2002/45695; U.S. Pat. Nos. 4,601,894; 4,687,757; 4,680,323; 4,994,276; and 3,538,214.

For preparation of stem cell (e.g., iPSC) grafts including activation factors stem cells can be substantially evenly distributed throughout a transplantation matrix with these factors. Transplantation matrices suitable for use in the body include e.g., the tissue adhesive compositions described in Petersen, et al., 2004, Gastrointestinal Endoscopy 60 (3): 327-333. A mixture of fibrin and thrombin can be particularly well-suited for stem cell (e.g., iPSC) delivery. Such mixtures are commercially available as fibrin glue products; e.g., a 50:50 mixture product from Sigma Chemicals. Stem cells (e.g., iPSC) can be evenly suspended in the tissue adhesive with activation factors, in particular embodiments, just prior to implantation.

Stem-cell activation factors may be loaded into mesoporous particles, e.g., mesoporous silica materials. The mesoporous particles can be a solvent extracted and/or a calcined material (see, e.g., Atluri, et al., 2008, Chemistry of Materials 20 (12), 3857-3866). Materials may be mixed with the desired amount of stem-cell activation factors in a solvent that will dissolve or partially dissolve the aforementioned factors. The mixture may be stirred, centrifuged, spray dried, or filtered after periods between 0.5 hours and 2 days at temperatures between 0-80° C. If the sample is stirred, the recovered solid typically contains between 20-49 wt % of factors within the pores of the mesoporous silica particle. Higher amounts can be obtained if the loading process is repeated several times. For additional detail regarding these delivery particles and methods, see U.S. Publication No. 2013/0315962.

U.S. Publication No. 2014/0308256 describes co-administration of stem-cell activation factors with stem cells in neural applications. For example, this disclosure teaches that stem cell survival and axonal growth may be enhanced by supplying a neural stem cell graft with an activation factor source. The source may be provided by co-administration or separate delivery of an activation factor, such as NT-3, BDNF, CTNF, NGF, NT-4/5, FGF, EGF and GDNF (including GDNF family neurotrophins such as neurturin). Concentrations between 1 to 100 ng/ml are usually sufficient and may be conveniently added to the cell graft composition, co-administered into the graft and/or administered within diffusion distance of the graft. When the neural stem cells are implanted at a target lesion site, suspended evenly in a transplantation matrix in the presence of at least one activation factor, the grafted neural stem cells differentiate, undergo axonal myelination, and establish synaptic contacts with host circuitry. Reciprocally, host axons penetrate grafts in the lesion site and establish putative synaptic contacts.

Stem-cell activation factors may also be provided by expression from a co-administered recombinant expression vector or from donor cells. Coding polynucleotides, precursors and promoters for a number of activation factors are known. For example, GenBank M61176 sets forth the coding sequence (mRNA) for BDNF; BDNF precursor is set forth at BF439589; and a BDNF specific promoter is set forth at E05933. A similar range of coding sequences for other activation factors are also available through GenBank and other publicly accessible nucleotide sequence databases.

Preparation of stem-cell activation factor-expressing donor cells (e.g., fibroblasts) may be as described in U.S. Pat. No. 6,451,306. Such cells may be co-grafted with stem cells (e.g., iPSC) but need not be included within a stem cell/transplantation matrix composition.

An additional method to control stem cell (e.g., iPSC) differentiation after transplantation is by controlled expression of transcription factors in the transplanted cells using drug-inducible regulation systems as described, e.g., in WO 2008/002250. For example, using the tetracycline gene regulation system to induce expression of the key transcription factor Runx1 in Sox10 expressing neural crest stem cells, specific differentiation of nociceptor neurons was observed in vivo after transplantation. See, e.g., Aldskogius, et al., 2009, Stem Cells 27:1592-603.

Another method to promote stem cell (e.g., iPSC) differentiation and survival after administration is through use of osmotic minipumps that provide stem-cell activation factors for improved survival, differentiation and function of transplanted cells.

In particular embodiments, co-transplantation of neural crest stem cells with pancreatic islets creates beneficial effects for both islets and stem cells with improved insulin secretion, increased proliferation of beta-cells and advanced differentiation of neural crest stem cells in the vicinity of islets. Olerud, et al., 2010, Diabetologia 53:396.

If the cells are derived from a heterologous (non-autologous/allogenic) source compared to the recipient subject, concomitant immunosuppression therapy is typically administered, e.g., administration of the immunosuppressive agent cyclosporine or FK506. However, due to the immature state of the iPSC, immunosuppressive therapy may not be required. Accordingly, in one embodiment, the iPSC can be administered to a recipient in the absence of immunomodulatory (e.g., immunsuppressive) therapy. Alternatively, the cells can be encapsulated in a membrane, which permits exchange of fluids but prevents cell/cell contact. Transplantation of microencapsulated cells is known in the art, e.g., Balladur et al., 1995, Surgery 117:189-94, 1995; and Dixit et al., 1992, Cell Transplantation 1:275-79.

In addition, the cells or tissue of the disclosure can be used, for example, to screen in vitro for the efficacy and/or cytotoxicity of compounds, allergens, growth/regulatory factors, pharmaceutical compounds, and the like on iPSC, to elucidate the mechanism of certain diseases by determining changes in the biological activity of the iPSC (e.g., changes in Klf, Oct4, Sox, Myc, SV40Tag, Nanog, Lin28 or any combination thereof expression or activity, proliferative capacity, adhesion), to study the mechanism by which drugs and/or growth factors operate to modulate iPSC biological activity (e.g., c Klf, Oct4, Sox, Myc, SV40Tag, Nanog, Lin28 or any combination thereof expression or activity), to diagnose and monitor cancer in a patient, for gene therapy, gene delivery or protein delivery; and to produce biologically active products.

The iPSC also can be used in the isolation and evaluation of factors associated with the differentiation and maturation of stem cells. Thus, the iPSC may be used in assays to determine the activity of media, such as conditioned media, evaluate fluids for cell growth activity, involvement with dedication of particular lineages, or the like. Various systems are applicable and can be designed to induced differentiation of the iPSC based upon various physiological stresses. The iPSC, progeny thereof, and tissues derived therefrom of the disclosure may be used in vitro to screen a wide variety of agents for effectiveness and cytotoxicity of pharmaceutical agents, growth/regulatory factors, anti-inflammatory agents, and the like. To this end, the cells or tissue cultures of the disclosure can be maintained in vitro and exposed to the agent to be tested. The activity of a cytotoxic agent can be measured by its ability to damage or kill stem cells or their progeny in culture. This can be assessed readily by staining techniques. The effect of growth/regulatory factors can be assessed by analyzing the number of living cells in vitro, e.g., by total cell counts, and differential cell counts. This can be accomplished using standard cytological and/or histological techniques, including the use of immunocytochemical techniques employing antibodies that define type-specific cellular antigens. The effect of various drugs on the cells of the disclosure can be assessed either in a suspension culture or in a three-dimensional system. In one aspect, the effect of a test agent on the iPSC can be analyzed.

For administration, therapeutically effective amounts (also referred to herein as doses) can be initially estimated based on results from in vitro assays and/or animal model studies. Such information can be used to more accurately determine useful doses in subjects of interest. The actual dose amount administered to a particular subject can be determined by a physician, veterinarian or researcher taking into account parameters such as physical and physiological factors including target, body weight, severity of condition, type of disease or injury, previous or concurrent therapeutic interventions, idiopathy of the subject and route of administration.

Therapeutically effective amounts of cell-based formulations can include 10⁴ to 10⁹ cells/kg body weight, or 10³ to 10¹¹ cells/kg body weight. Therapeutically effective amounts to administer can include greater than 10² cells, greater than 10³ cells, greater than 10⁴ cells, greater than 10⁵ cells, greater than 10⁶ cells, greater than 10⁷ cells, greater than 10⁸ cells, greater than 10⁹ cells, greater than 10¹⁰ cells, or greater than 10¹¹.

Therapeutically effective amounts can be achieved by administering single or multiple doses during the course of a treatment regimen (e.g., daily, every other day, every 3 days, every 4 days, every 5 days, every 6 days, weekly, every 2 weeks, every 3 weeks, monthly, every 2 months, every 3 months, every 4 months, every 5 months, every 6 months, every 7 months, every 8 months, every 9 months, every 10 months, every 11 months or yearly). In particular embodiments, the treatment protocol may be dictated by a clinical trial protocol or an FDA-approved treatment protocol.

Therapeutically effective amounts can be administered by, e.g., injection, infusion, perfusion, or lavage. Routes of administration can include bolus intravenous, intradermal, intraarterial, intraparenteral, intranodal, intralymphatic, intraperitoneal, intralesional, intraprostatic, intravaginal, intrarectal, topical, intrathecal, intratumoral, intramuscular, intravesicular, and/or subcutaneous administration.

In certain embodiments, iPSC are administered to a patient in conjunction with (e.g., before, simultaneously or following) any number of relevant treatment modalities. In particular embodiments, cells may be used in combination with chemotherapy, radiation, immunosuppressive agents, such as cyclosporin, azathioprine, methotrexate, mycophenolate, and FK506, antibodies, or other immunoablative agents such as CAM PATH, anti-CD3 antibodies or other antibody therapies, cytoxin, fludaribine, cyclosporin, FK506, rapamycin, mycoplienolic acid, steroids, FR901228, cytokines, and irradiation.

The Exemplary Embodiments and Experimental Examples below are included to demonstrate particular embodiments of the disclosure. Those of ordinary skill in the art should recognize in light of the present disclosure that many changes can be made to the specific embodiments disclosed herein and still obtain a like or similar result without departing from the spirit and scope of the disclosure.

### (xiii) EXEMPLARY EMBODIMENTS

1. An engineered expression construct (EEC) having a reprogramming factor (RF) coding sequence operably linked to a 5′ untranslated region (UTR) including the sequence as set forth in SEQ ID NOs: 1, 2, 3, or 4 and a 3′ UTR including the sequence as set forth in SEQ ID NOs: 28-45.
2. An engineered expression construct (EEC) having a 5′ untranslated region (UTR) operably linked to a reprogramming factor (RF) coding sequence, wherein the 5′ UTR has the sequence as set forth in CAUACUCA in between a minimal promoter and a Kozak sequence.
3. The EEC of embodiment 2, wherein the minimal promoter is a T7 promoter.
4. The EEC of embodiment 3, wherein the T7 promoter has the sequence as set forth in GGGAGA.
5. The EEC of any of embodiments 2-4, wherein the Kozak sequence has the sequence as set forth in GCCRCC, wherein R is A or G.
6. The EEC of any of embodiments 2-5, wherein the 5′ UTR further includes a start codon.
7. The EEC of any of embodiments 2-6, wherein the 5′ UTR has
(i) the sequence as set forth in SEQ ID NO: 1 operably linked to a start codon or
(ii) the sequence as set forth in SEQ ID NO: 2 operably linked to a start codon.
   8. The EEC of embodiment 7, wherein the sequence as set forth in SEQ ID NO: 1 operably linked to a start codon has the sequence as set forth in SEQ ID NO: 3.
   9. The EEC of embodiment 7, wherein the sequence as set forth in SEQ ID NO: 2 operably linked to a start codon has the sequence as set forth in SEQ ID NO: 4.
   10. The EEC of any of embodiments 2-9, wherein the 5′ UTR is less than 30 nucleotides.
   11. The EEC of any of embodiments 2-10, further including a 3′ UTR.
   12. The EEC of embodiment 11, wherein the 3′ UTR includes a stop codon, spacer, and a stem loop structure.
   13. The EEC of embodiment 12, wherein the stop codon has the sequence UAA, UGA, or UAG.
   14. The EEC of embodiments 12 or 13, wherein the spacer has the sequence [N₁₋₃]AUA or [N₁₋₃]AAA.
   15. The EEC of any of embodiments 12-14, wherein the spacer has the sequence UGCAUA or UGCAAA.
   16. The EEC of any of embodiments 12-15, wherein the stem loop structure has hybridizing sequences as set forth in a) CCUC and GAGG or b) CUCC and GGAG.
   17. The EEC of any of embodiments 12-16, wherein the stem loop structure has a loop segment having at least 7 nucleotides.
   18. The EEC of any of embodiments 12-17, wherein the stem loop structure has a loop segment having 7-15 nucleotides.
   19. The EEC of any of embodiments 12-18, wherein the stem loop structure has a loop segment having the sequence as set forth in UAACGGUCUU (SEQ ID NO: 5).
   20. The EEC of any of embodiments 11-19, wherein the 3′ UTR is less than 30 nucleotides.
   21. The EEC of any of embodiments 11-20, wherein the 3′ UTR further includes a polyadenine (polyA) tail.
   22. The EEC of embodiment 21, wherein the polyA tail has 60 residues or less.
   23. The EEC of embodiments 21 or 22, wherein the polyA tail has 40 residues.
   24. The EEC of any of embodiments 11-24, wherein the 3′ UTR has the sequence as set forth in SEQ ID NO: 28, SEQ ID NO: 29, SEQ ID NO: 30, SEQ ID NO: 31, SEQ ID NO: 32, or SEQ ID NO: 33.
   25. The EEC of any of embodiments 11-24, wherein the 3′ UTR has the sequence as set forth in SEQ ID NO: 34, SEQ ID NO: 35, or SEQ ID NO: 36.
   26. The EEC of any of embodiments 11-24, wherein the 3′ UTR has the sequence as set forth in SEQ ID NO: 37, SEQ ID NO: 38, SEQ ID NO: 39, SEQ ID NO: 40, SEQ ID NO: 41, SEQ ID NO: 42, SEQ ID NO: 43, SEQ ID NO: 44, or SEQ ID NO: 45.
   27. The EEC of any of embodiments 2-26, wherein the EEC includes in vitro-synthesized messenger RNA (mRNA).
   28. The EEC of any of embodiments 2-27, wherein the EEC does not include modified nucleosides.
   29. The EEC of any of embodiments 2-28, wherein the EEC does not include microRNA binding sites.
   30. The EEC of any of embodiments 2-29, wherein the further includes a coding sequence encoding an immune evading factor.
   31. The EEC of embodiment 30, wherein the immune evading factor includes B18R, E3, K3, NS1, or ORF8.
   32. The EEC of any of embodiments 2-29, wherein the EEC does not encode immune evading factors.
   33. The EEC of any of embodiments 2-32, formulated for administration to a subject.
   34. The EEC of any of embodiments 2-33, wherein the reprograming factor includes Oct4, Sox (e.g., Sox1, Sox2, Sox3, Sox15, and/or Sox18), Klf (e.g., Klf4, Klf1, and/or Klf5), Nanog, Myc (e.g., cMyc, N-Myc, and/or L-Myc), SV40 large T antigen (SV40Tag), estrogen-related receptor beta (Essrb), and/or Lin28.
   35. An enhancer sequence including the sequence as set forth in CAUACUCA operably linked to a reprogramming factor coding sequence.
   36. An engineered expression construct (EEC) having 1, 2, 3, 4, or 5 copies of the sequence as set forth in CAUACUCA operably linked to a reprogramming factor coding sequence.
   37. The EEC of embodiment 36, wherein the enhancer sequence is operably linked to a promoter.
   38. The EEC of embodiment 37 wherein the promoter is a minimal promoter.
   39. The EEC of embodiment 38, wherein the minimal promoter is a T7 promoter.
   40. The EEC of embodiment 39, wherein the T7 promoter includes the sequence: GGAGA.
   41. The EEC of any of embodiments 36-40, wherein the reprograming factor includes Oct4, Sox (e.g., Sox1, Sox2, Sox3, Sox15, and/or Sox18), Klf (e.g., Klf4, Klf1, and/or Klf5), Nanog, Myc (e.g., cMyc, N-Myc, and/or L-Myc), SV40 large T antigen (SV40Tag), estrogen-related receptor beta (Essrb), and/or Lin28.
   42. An engineered expression construct (EEC) including an in vitro-synthesized RNA including a reprogramming factor (RF) coding sequence within an open reading frame that encodes one or more RF for translation in a mammalian cell, wherein said in vitro-synthesized RNA further includes one of a 5′ untranslated region including CAUACUCA and a 3′ untranslated region including one of SEQ ID NO: 28, SEQ ID NO: 29, SEQ ID NO: 30, SEQ ID NO: 31, SEQ ID NO: 32, or SEQ ID NO: 33.
   43. The EEC of embodiment 42, wherein the reprograming factor includes Oct4, Sox (e.g., Sox1, Sox2, Sox3, Sox15, and/or Sox18), Klf (e.g., Klf4, Klf1, and/or Klf5), Nanog, Myc (e.g., cMyc, N-Myc, and/or L-Myc), SV40 large T antigen (SV40Tag), estrogen-related receptor beta (Essrb), and/or Lin28.
   44. An engineered expression construct (EEC) including an in vitro-synthesized RNA including a reprogramming factor (RF) coding sequence within an open reading frame that encodes one or more RF for translation in a mammalian cell, wherein said in vitro-synthesized RNA further includes one of a 5′ untranslated region including SEQ ID NO: 1, or SEQ ID NO: 2 and a 3′ untranslated region including SEQ ID NO: 28, SEQ ID NO: 29, SEQ ID NO: 30, SEQ ID NO: 31, SEQ ID NO: 32, or SEQ ID NO: 33.
   45. The EEC of embodiment 44, wherein the reprograming factor includes Oct4, Sox (e.g., Sox1, Sox2, Sox3, Sox15, and/or Sox18), Klf (e.g., Klf4, Klf1, and/or Klf5), Nanog, Myc (e.g., cMyc, N-Myc, and/or L-Myc), SV40 large T antigen (SV40Tag), estrogen-related receptor beta (Essrb), and/or Lin28.
   46. An engineered expression construct (EEC) including an in vitro-synthesized RNA including an open reading frame that encodes one or more reprogramming factors (RF) for translation in a mammalian cell, wherein said in vitro-synthesized RNA further includes a 5′ untranslated region including a T7 promoter, the sequence as set forth in CAUACUCA, and a Kozak sequence.
   47. The EEC of embodiment 46, wherein the one or more RF include Oct4, Sox (e.g., Sox1, Sox2, Sox3, Sox15, and/or Sox18), Klf (e.g., Klf4, Klf1, and/or Klf5), Nanog, Myc (e.g., cMyc, N-Myc, and/or L-Myc), SV40 large T antigen (SV40Tag), estrogen-related receptor beta (Essrb), and/or Lin28.
   48. The EEC of embodiments 46 or 47, wherein the T7 promoter is selected from a T7 Class III promoter.
   49. The EEC of embodiment 48, wherein the T7 Class III promoter includes the sequence as set forth in GGGAGA.
   50. The EEC of any of embodiments 46-49, wherein the Kozak sequence includes the sequence as set forth in GCCRCC, wherein R is A or G.
   51. The EEC of any of embodiments 46-50, further including a start codon.
   52. An engineered expression construct (EEC) including an in vitro-synthesized RNA including an open reading frame that encodes one or more RF for translation in a mammalian cell, wherein said in vitro-synthesized RNA includes a 3′ untranslated region including SEQ ID NO: 28, SEQ ID NO: 29, SEQ ID NO: 30, SEQ ID NO: 31, SEQ ID NO: 32, or SEQ ID NO: 33 and a stop codon.
   53. The EEC of embodiment 52, wherein the one or more RF include Oct4, Sox (e.g., Sox1, Sox2, Sox3, Sox15, and/or Sox18), Klf (e.g., Klf4, Klf1, and/or Klf5), Nanog, Myc (e.g., cMyc, N-Myc, and/or L-Myc), SV40 large T antigen (SV40Tag), estrogen-related receptor beta (Essrb), and/or Lin28.
   54. The engineered expression construct of embodiments 52 or 53, wherein the stop codon is UAA, UAG, or UGA.
   55. An engineered expression construct (EEC) including an in vitro-synthesized RNA including an open reading frame that encodes one or more RFs for translation in a mammalian cell, wherein said in vitro-synthesized RNA includes a 3′ untranslated region including either a) CCUC and GAGG or b) CUCC and GGAG wherein either set of the 3′ untranslated region sequences is separated by no fewer than seven nucleotides.
   56. The EEC of embodiment 55, wherein the one or more RF include Oct4, Sox (e.g., Sox1, Sox2, Sox3, Sox15, and/or Sox18), Klf (e.g., Klf4, Klf1, and/or Klf5), Nanog, Myc (e.g., cMyc, N-Myc, and/or L-Myc), SV40 large T antigen (SV40Tag), estrogen-related receptor beta (Essrb), and/or Lin28.
   57. The EEC of embodiments 55 or 56, further including a spacer.
   58. The EEC of embodiment 57, wherein the spacer includes the sequence N₁₋₃]AUA or [N₁₋₃]AAA. 59. The EEC of embodiments 57 or 58, wherein the spacer includes the sequence UGCAUA or UGCAAA.
   60. An engineered expression construct (EEC) having an open reading frame (ORF) and a 5′ untranslated region (UTR) and/or a 3′UTR, wherein
the ORF encodes a reprogramming factor (RF) and/or an immune evading factor (IEF);
the 5′ UTR includes a minimal promoter, the sequence as set forth in CAUACUCA, and a Kozak sequence;
the 3′UTR includes a stop codon, spacer, and a stem loop structure.
   61. The EEC of embodiment 60, wherein the minimal promoter is a T7 promoter.
   62. The EEC of embodiment 61, wherein the T7 promoter has the sequence as set forth in GGGAGA.
   63. The EEC of any of embodiments 60-62, wherein the Kozak sequence has the sequence as set forth in GCCRCC, wherein R is A or G.
   64. The EEC of any of embodiments 60-63, wherein the 5′ UTR further includes a start codon.
   65. The EEC of any of embodiments 60-64, wherein the 5′ UTR has
(iii) the sequence as set forth in SEQ ID NO: 1 operably linked to a start codon or
(iv) the sequence as set forth in SEQ ID NO: 2 operably linked to a start codon.
   66. The EEC of embodiment 65, wherein the sequence as set forth in SEQ ID NO: 1 operably linked to a start codon has the sequence as set forth in SEQ ID NO: 3.
   67. The EEC of embodiment 65, wherein the sequence as set forth in SEQ ID NO: 2 operably linked to a start codon has the sequence as set forth in SEQ ID NO: 4.
   68. The EEC of any of embodiments 60-67, wherein the 3′ UTR further includes a stop codon.
   69. The EEC of embodiment 68, wherein the stop codon has the sequence UAA, UGA, or UAG.
   70. The EEC of any of embodiments 60-69, wherein the spacer has the sequence [N₁₋₃]AUA or [N₁₋₃]AAA.
   71. The EEC of any of embodiments 60-70, wherein the spacer has the sequence UGCAUA or UGCAAA.
   72. The EEC of embodiment 60-71, wherein the stem loop structure has hybridizing sequences as set forth in a) CCUC and GAGG or b) CUCC and GGAG.
   73. The EEC of any of embodiments 60-72, wherein the stem loop structure has a loop segment having at least 7 nucleotides.
   74. The EEC of any of embodiments 60-73, wherein the stem loop structure has a loop segment having 7-15 nucleotides.
   75. The EEC of any of embodiments 60-74, wherein the stem loop structure has a loop segment having the sequence as set forth in UAACGGUCUU (SEQ ID NO: 5).
   76. The EEC of any of embodiments 60-75, wherein the 3′ UTR further includes a polyadenine (polyA) tail.
   77. The EEC of any of embodiments 60-76, wherein the 3′ UTR has the sequence as set forth in SEQ ID NO: 28, SEQ ID NO: 29, SEQ ID NO: 30, SEQ ID NO: 31, SEQ ID NO: 32, or SEQ ID NO: 33.
   78. The EEC of any of embodiments 60-77, wherein the 3′ UTR has the sequence as set forth in SEQ ID NO: 34, SEQ ID NO: 35, or SEQ ID NO: 36.
   79. The EEC of any of embodiments 60-78, wherein the 3′ UTR has the sequence as set forth in SEQ ID NO: 37, SEQ ID NO: 38, SEQ ID NO: 39, SEQ ID NO: 40, SEQ ID NO: 41, SEQ ID NO: 42, SEQ ID NO: 43, SEQ ID NO: 44, or SEQ ID NO: 45.
   80. The EEC of any of embodiments 60-79, wherein the reprograming factor includes Oct4, Sox (e.g., Sox1, Sox2, Sox3, Sox15, and/or Sox18), Klf (e.g., Klf4, Klf1, and/or Klf5), Nanog, Myc (e.g., cMyc, N-Myc, and/or L-Myc), SV40 large T antigen (SV40Tag), estrogen-related receptor beta (Essrb), and/or Lin28.
   81. The EEC of embodiment 80, wherein the EEC includes the sequence as set forth in SEQ ID NO: 84, SEQ ID NO: 86, SEQ ID NO: 89, SEQ ID NO: 85, SEQ ID NO: 87, SEQ ID NO: 90, SEQ ID NO: 88, SEQ ID NO: 105, SEQ ID NO: 106, or SEQ ID NO: 107.
   82. The EEC of any of embodiments 60-81, wherein the IEF includes B18R, E3, and/or K3.
   83. The EEC of embodiment 82, wherein the EEC includes the sequence as set forth in SEQ ID NO: 91.
   84. The EEC of embodiments 82 or 83, wherein the EEC includes the sequence as set forth in SEQ ID NO: 92.
   85. The EEC of any of embodiment 82-84, wherein the EEC includes the sequence as set forth in SEQ ID NO: 93.
   86. A method for reprogramming somatic cells into induced pluripotent stem cells (iPSC) including: contacting somatic cells with the EEC of any of embodiments 1-84.
   87. A method for reprogramming somatic cells into induced pluripotent stem cells (iPSC) including: contacting somatic cells with an engineered expression construct (EEC) encoding a reprogramming factor (RF) operably linked to
i) a 5′ untranslated region (UTR) including a minimal promoter, a mini-enhancer, and a Kozak sequence; or
ii) a 3′ UTR including a spacer and a stem loop structure.
   88. The method of embodiment 87, wherein the RF includes Oct4, Sox (e.g., Sox1, Sox2, Sox3, Sox15, and/or Sox18), Klf (e.g., Klf4, Klf1, and/or Klf5), Nanog, Myc (e.g., cMyc, N-Myc, and/or L-Myc), SV40 large T antigen (SV40Tag), estrogen-related receptor beta (Essrb), and/or Lin28.
   89. The method of embodiments 87 or 88, wherein the minimal promoter includes a T7 promoter.
   90. The method of embodiment 89, wherein the T7 promoter includes the sequence as set forth in GGGAGA.
   91. The method of any of embodiments 87-90, wherein the mini-enhancer includes the sequence as set forth in CAUACUCA.
   92. The method of any of embodiments 87-91, wherein the Kozak sequence includes the sequence as set forth in GCCRCC wherein R is A or G.
   93. The method of embodiment 92, wherein the Kozak sequence includes the sequence as set forth in GCCACC or GCCGCC.
   94. The method of any of embodiments 87-93, wherein the 5′ UTR includes the sequence as set forth in SEQ ID NO: 1 or SEQ ID NO: 2.
   95. The method of any of embodiments 87-94, wherein the 5′ UTR further includes a start codon.
   96. The method of any of embodiments 87-95, wherein the 5′ UTR includes the sequence as set forth in SEQ ID NO: 3 or SEQ ID NO: 4.
   97. The method of any of embodiments 87-96, wherein the spacer includes a sequence as set forth in [N₁₋₃]AUA or [N₁₋₃]AAA.
   98. The method of any of embodiments 87-97, wherein the spacer includes a sequence as set forth in UGCAUA or UGCAAA.
   99. The method of any of embodiments 87-98, wherein the stem loop structure includes two hybridizing sequences and a loop segment.
   100. The method of embodiment 99, wherein the two hybridizing sequences include i) CCUC and GAGG, or ii) CUCC and GGAG.
   101. The method of embodiments 99 or 100, wherein the loop segment includes at least 7 nucleotides.
   102. The method of any of embodiments 99-101, wherein the loop segment includes 7-15 nucleotides.
   103. The method of any of embodiments 99-102, wherein the loop segment includes the sequence as set forth in SEQ ID NO: 5.
   104. The method of any of embodiments 87-103, wherein the stem loop structure includes the sequence as set forth in SEQ ID NO: 8, SEQ ID NO: 9, SEQ ID NO: 10, SEQ ID NO: 11, SEQ ID NO: 12, SEQ ID NO: 13, SEQ ID NO: 14, SEQ ID NO: 15, SEQ ID NO: 16, SEQ ID NO: 17, SEQ ID NO: 18, SEQ ID NO: 19, SEQ ID NO: 20, SEQ ID NO: 21, SEQ ID NO: 22, SEQ ID NO: 23, SEQ ID NO: 24, SEQ ID NO: 25, SEQ ID NO: 26, or SEQ ID NO: 27.
   105. The method of any of embodiments 87-104, wherein the 3′ UTR includes the sequence as set forth in SEQ ID NO: 28, SEQ ID NO: 29, SEQ ID NO: 30, SEQ ID NO: 31, SEQ ID NO: 32, SEQ ID NO: 33, SEQ ID NO: 34, SEQ ID NO: 35, or SEQ ID NO: 36.
   106. The method of any of embodiments 87-105, wherein the 3′ UTR further includes a stop codon.
   107. The method of embodiment 106, wherein the stop codon includes the sequence as set forth in UAA, UAG, or UGA.
   108. The method of any of embodiments 87-107, wherein the 3′ UTR includes the sequence as set forth in SEQ ID NO: 37, SEQ ID NO: 38, SEQ ID NO: 39, SEQ ID NO: 40, SEQ ID NO: 41, SEQ ID NO: 42, SEQ ID NO: 43, SEQ ID NO: 44, or SEQ ID NO: 45.
   109. The method of any of embodiments 87-108, wherein the somatic cells include mammalian cells.
   110. The method of any of embodiments 87-109, wherein the somatic cells include somatic cells.
   111. The method of any of embodiments 87-110, wherein the somatic cells include fibroblasts, hepatocytes, myoblasts, neurons, osteoblasts, osteoclasts, kidney cells, immune cells, or non-pluripotent stem cells.
   112. The method of embodiment 111, wherein the fibroblasts include adult dermal fibroblasts or human foreskin fibroblasts.
   113. The method of embodiment 111, wherein the kidney cells include HEK293 cells.
   114. The method of embodiment 111, wherein the immune cells include T cells.
   115. The method of embodiment 111, wherein the non-pluripotent stem cells include hematopoietic stem cells or mesenchymal stem cells.
   116. The method of embodiment 111, wherein the hematopoietic stem cells include CD34+ cells.
   117. The method of any of embodiments 87-116, further including electroporating the somatic cells with mRNA encoding an immune evasion factor (IEF).
   118. The method of embodiment 117, wherein the IEF includes B18R, E3, K3, NS1, or ORF8.
   119. The method of embodiments 117 or 118, wherein the mRNA encoding the IEF includes a 5′UTR including the sequence as set forth in SEQ ID NO: 1 and a 3′UTR including the sequence as set forth in SEQ ID NO: 34.
   120. The method of embodiment 119, wherein the mRNA encoding the IEF includes the sequence as set forth in SEQ ID NO: 91, SEQ ID NO: 92, or SEQ ID NO: 93.
   121. The method of any of embodiments 87-120, further including contacting the somatic cells with miRNA.
   122. The method of embodiment 121, wherein the miRNA include miR-302a, miR-302b, miR-302c, miR-302d, and miR-367.
   123. The method of embodiments 121 or 122, wherein the miR-302a includes the sequence as set forth in SEQ ID NO: 64.
   124. The method of any of embodiments 121-123, wherein the miR-302b includes the sequence as set forth in SEQ ID NO: 65.
   125. The method of any of embodiments 121-124, wherein the miR-302a includes the sequence as set forth in SEQ ID NO: 66.
   126. The method of any of embodiments 121-125, wherein the miR-302a includes the sequence as set forth in SEQ ID NO: 67.
   127. The method of any of embodiments 121-126, wherein the miR-367 includes the sequence as set forth in SEQ ID NO: 68.
   128. The method of any of embodiments 87-127, further including contacting the somatic cells with an IEF protein.
   129. The method of embodiment 128, wherein the IEF protein includes B18R, E3, K3, NS1, or ORF8.
   130. The method of any of embodiments 87-129, wherein the method includes contacting the isolated somatic cells with the EEC daily for 10 days or less, 9 days or less, 8 days or less, 7 days or less, 6 days or less, 5 days or less, 4 days or less.
   131. The method of any of embodiments 87-130, wherein the method includes contacting the isolated somatic cells with the EEC daily for 4 days.
   132. The method of any of embodiments 87-131, wherein the method does not use feeder cells.
   133. An induced pluripotent stem cell (iPSC) made according to the method of any of embodiments 86-132.
   134. An engineered expression construct (EEC) including the sequence as set forth in SEQ ID NO: 71, SEQ ID NO: 72, SEQ ID NO: 73, SEQ ID NO: 74, SEQ ID NO: 75, SEQ ID NO: 76, SEQ ID NO: 77, SEQ ID NO: 78, SEQ ID NO: 79, SEQ ID NO: 80, SEQ ID NO: 81, SEQ ID NO: 84, SEQ ID NO: 85, SEQ ID NO: 86, SEQ ID NO: 87, SEQ ID NO: 88, SEQ ID NO: 89, SEQ ID NO: 90, SEQ ID NO: 91, SEQ ID NO: 92, SEQ ID NO: 93, SEQ ID NO: 10⁵, SEQ ID NO: 10⁶, or SEQ ID NO: 107.
   135. A cDNA encoding the EEC of any of embodiments 1-85 or 134.

### (xiv) EXPERIMENTAL EXAMPLES

Example 1. Materials and Methods. Untranslated Region (UTR) Design and Structure Prediction. The minimal transcription and translation elements (for example, the unique 5′ UTR enhancer (e.g., CAUACUCA), the T7 hexamer, and the Kozak sequence, all described herein), which are four to ten nucleotides in length, are assembled to construct the UTRs of this example. Based on the stem loop features, synthetic 3′ sequences (e.g., SEQ ID NO: 34) were assembled for testing. The secondary structure prediction webservers (rna.urmc.rochester.edu/RNAstructureWeb/) were utilized with default parameters to examine the likelihood of stem loop secondary structure being formed from the various UTR sequences.

TABLE 4
List of PCR Primers

| Universal | | SEQ ID |
|---|---|---|
| PCR | Sequence | NO: |
| Forward | GACTCCTAATACGACTCACTATAGGGAGAATG | 48 |
| (No UTR) | | |
| Forward | GACTCCTAATACGACTCACTATAGGGAGAGCCACCATG | 49 |
| (Kozak) | | |
| Forward | GACTCCTAATACGACTCACTATAGGGAGACATACTCAGCCACCA | 50 |
| (5′ UTR) | TG | |
| Reverse | TTTTTTTTTTTTTTTTTTTTTTTTTTTTTTTTTTTTTTTTCCTCAAGAC | 51 |
| (3′ UTR-A) | CGTTAGAGGTATGCA | |
| Reverse | TTTTTTTTTTTTTTTTTTTTTTTTTTTTTTTTTTTTTTTTGGAGAAGA | 52 |
| (3′ UTR-B) | CCGTTACTCCTATGCA | |
| Reverse | TTTTTTTTTTTTTTTTTTTTTTTTTTTTTTTTTTTTTTTTGGAGAAGA | 53 |
| (3′ UTR-C) | CCGTTACTCCTTTGCA | |

mRNA Synthesis. gBlocks™ Gene fragments (Integrated DNA Technologies, Coralville, IA) were PCR-amplified using the Q5® Hot Start High-Fidelity DNA Polymerase (New England Biolabs, Ipswich, MA) to generate the cDNA template for the in vitro transcription (IVT). The T7 RNA polymerase promoter, 5′ and 3′ UTRs (SEQ ID NO: 1 and SEQ ID NO: 34, respectively) and PolyA tail sequences were introduced via custom forward and reverse primers. The cDNA template was transcribed using the T7 RNA Polymerase (HiScribe™ T7 High Yield RNA Synthesis Kit, New England Biolabs) followed by DNase I treatment (New England Biolabs). Resulting mRNA was capped using the Vaccinia Virus Capping Enzyme and 2′-O-methylation (New England Biolabs). Following DNase I treatment, the mRNAs were quantified and stored accordingly.

miRNA. To enhance reprogramming, 400 ng of a miRNA mixture composed of mature duplex miRNAs 302a-d and 367 [0.4 μM each] (Qiagen, Hilden, Germany or Genepharma, Suzhou, China) were added to the mRNA reprogramming cocktail. See Table 3 for miRNA sequences.

Cell Culture. Early passage fibroblasts, either Human Foreskin Fibroblasts Millipore Sigma, Burlington, MA) or adult Human Dermal Fibroblasts (BiolVT) were cultured in FibroGRO Xeno-Free medium (Merck KGAA, Darmstadt, Germany) at 37° C., 5% CO2. Induced pluripotent stem cells (iPSC) derived from fibroblasts (human foreskin and adult dermal) via reprogramming were cultured in TeSR E8 medium (StemCell Technologies, Vancouver, CA) or NutriStem™ hPSC XF Culture Medium (Biological Industries, Haemek, Israel) on iMatrix-511 substrate (Reprocell) at 37° C., 5% CO2. HEK293 (ATCC® CRL-1573™) cells were obtained from the American Type Culture Collection (ATCC). All cells were maintained at 37° C. with 5% CO2. HEK293 media includes Eagle's Minimum Essential Medium (EMEM) (ATCC® 30-2003™) with 10% fetal bovine serum (FBS).

Early passage human CD34+ cells (StemCell Technologies) were cultured in StemSpan SFEM medium (StemCell Technologies) supplemented with cytokines: 100 ng/ml SCF, 100 ng/ml FLT3, 50 ng/ml TPO, 20 ng/ml IL6, 10 ng/ml IL3 (Peprotech).

Early passage Human Mesenchymal Stem/Stromal Cells (hMSC, RoosterBio) were cultured in RoosterNourish-MSC culture medium (RoosterBio) following the manufacturer's instructions.

Reprogramming. Human Foreskin Fibroblasts (HFF), human Adult Dermal Fibroblasts, human Mesenchymal Stromal Cells and CD34+ cells were reprogrammed using the Reprogramming Factors (RF) and maintained in a 5% CO₂, 5% O₂ environment until colony isolation stage.

Early passage fibroblasts (human foreskin and adult dermal) and Mesencymal Stromal Cells were plated onto iMatrix-511 substrate in NutriStem™ hPSC XF Culture Medium (Biological Industries) at optimized seeding density and transfected daily over 4 consecutive days with Lipofectamine™ RNAiMAX (ThermoFisher Scientific, Waltham, MA) and the Reprogramming Cocktail containing: 800 ng mRNA of RF Oct4, Sox2, Klf4, cMyc, Nanog, Lin28 (control and proprietary synthesized), 600 ng mRNA of Immune Evasion Factors (IEF) B18R, E3, K3 (control and synthesized) and 400 ng microRNAs (302a-d, 367). In some embodiments, prior to plating the early passage fibroblasts were electroporated with the IEF, 600 ng mRNA E3, K3, and B18R. In other embodiments, 6 hours prior to transfecting the cells with the Reprogramming Cocktail, the cells were transfected with the IEF, mRNA E3, K3, and B18R, using lipofection. In other embodiments the reprogramming medium was supplemented with 200 ng/ml purified B18R protein (ThermoFisher Scientific, Waltham, MA) for the duration of the transfection regiment, in place of the transfection of the IEF mRNA. Emergent iPSC colonies were manually picked or bulked-passaged and expanded in NutriStem™ hPSC XF Culture Medium (Biological Industries, Haemek, Israel) on iMatrix-511 substrate, and characterized for pluripotency and differentiation markers by flow cytometry.

For reprogramming of CD34+ cells, cells are collected by centrifugation, transfected with the Reprogramming Cocktail by repeated electroporation using the Neon Electroporation system (ThermoFisher Scientific) and cultured in SFEM medium supplemented with cytokines. After the last electroporation, the cells are plated on iMatrix-511, and cultured in NutriStem™ hPSC XF Culture Medium once emergent iPSC colonies are apparent.

Transfection and Electroporation. Transfection of cells was performed with either jetMessenger® (PolyPlus Transfection, Illkirch Graffenstaden, France), Lipofectamine™ RNAiMAX or Lipofectamine™ Messenger Max (ThermoFisher Scientific) using optimized ratios of delivery reagent to mRNA (100 ng-1000 ng). Electroporation of cells was performed with the Neon Electroporation system (ThermoFisher Scientific) using settings optimized for each individual cell line according to the manufacturer's recommendations, to minimize cell death and ensure optimal levels of protein expression.

Flow Cytometry Analysis of Protein Expression. Flow cytometry analysis of pluripotency and differentiation markers (OCT4, SSEA4, SSEA1) was performed using the Human and Mouse Pluripotent Stem Cell Analysis Kit (BD Biosciences, Franklin Lakes, NJ) following the manufacturer's instructions. For flow cytometry analysis of RF protein expression, cell samples were fixed for 20 minutes with 4% paraformaldehide buffer (ThermoFisher Scientific), permeabilized on ice with BD Phosflow Perm Buffer III (BD Biosciences), followed by incubation for 30 minutes at room temperature with fluorophore-conjugated primary antibodies and corresponding isotype controls to verify for non-specific antibody binding (Oct4, Sox2 and Nanog antibodies from Cell Signaling Technology, Lin28 antibody from BD Pharmingen (San Diego, CA)). Sample acquisition was performed using a CytoFlex flow cytometer (Beckman Coulter, Brea, CA) and data analysis was performed with the FlowJo software (BD Biosciences).

Microscopy. Brightfield and phase contrast images of cells were acquired using a Keyence BZ-X800 Microscope, using either a 4× or 10× objective. For measurement of TRA-1-60 protein expression (stem cell marker), live cells were stained using a TRA-1-60 Alexa Fluor™ 488 antibody (ThermoFisher Scientific) following the manufacturer's instructions and immediately imaged using the Keyence BZ-X800 Microscope (4× objective) using a FITC filter. For measurement of Alkaline Phosphatase activity by iPSC, cells were fixed and stained using Alkaline Phosphatase Staining Kit (Abcam) per the manufacturer's instructions.

Example 2. The engineered mRNA containing the unique 5′ UTR (SEQ ID NO: 1) sequences resulted in increased RF expression when compared to mRNA using modified nucleotides when transfected into fibroblasts.

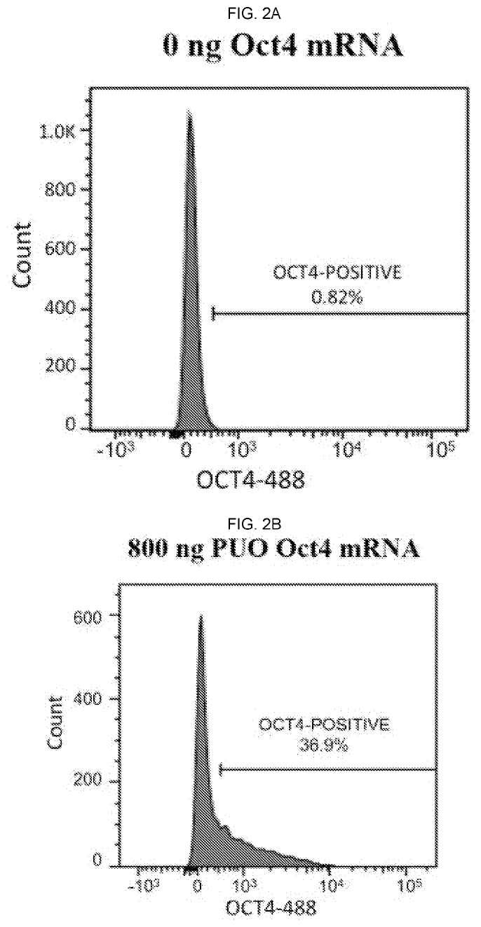

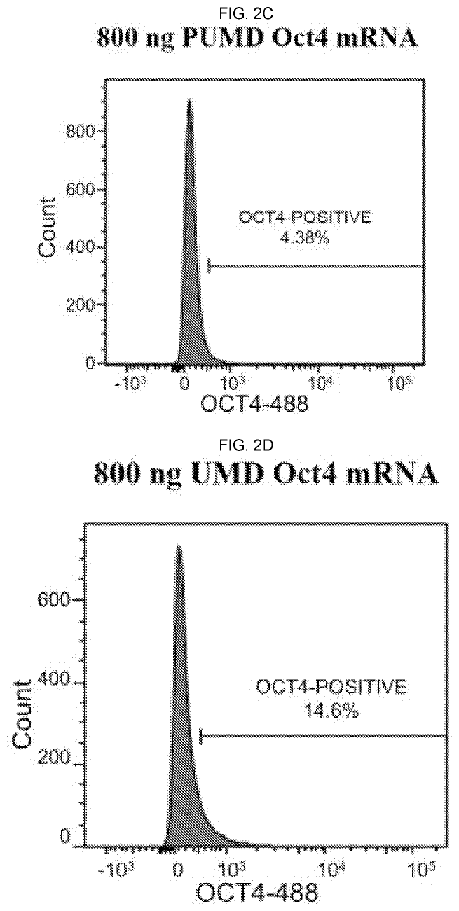

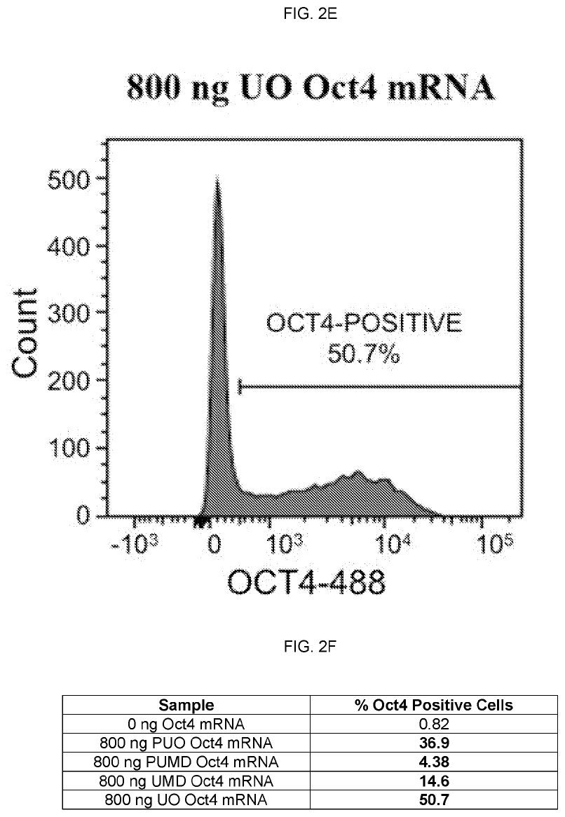

To compare the level of RF produced from the engineered mRNA with that of mRNA using modified nucleotides (N1-methyl-pseudouridine), several Oct4 expressing mRNA constructs were transfected into human foreskin fibroblasts, including mRNA Oct4 using unmodified/regular nucleotides (UO), mRNA MyoD-Oct4 using unmodified/regular nucleotides (UMD), modified mRNA Oct4 using N1-methyl-pseudouridine (PUO), and modified mRNA MyoD-Oct4 using N1-methyl-pseudouridine (PUMD). MyOD is the N-terminal MyoD transactivation domain, which when fused to OCT4 gene can enhance reprogramming. As shown in FIGS. 2A-2F, when using 800 ng of mRNA per transfection, Oct4 expression was the highest using the mRNA from unmodified nucleotides (UO). The unmodified, engineered transcripts using 800 ng of mRNA resulted in the highest percentage of Oct4-positive cells at 50.7%. Further, as shown in FIGS. 2A-2F, the percentage of Oct4-positive cells was significantly lower using mRNAs having the modified nucleoside N1-methyl-pseudouridine (PUO and PUMD) than the engineered mRNAs (36.9% compared to 50.7%).

This result shows that the engineered 5′ and 3′ UTRs result in higher levels of RF expression than transcripts made with modified nucleotides, like N1-methyl-pseudouridine.

Example 3. Transfection of synthetic mRNA results in increased RF expression in human foreskin fibroblasts and HEK293.

To test the ability of synthetic mRNA to increase RF expression, human foreskin fibroblasts were transfected with increasing quantities of mRNA for Oct4, Sox2, Lin28 and Nanog (200 ng-800 ng) using the jetMessenger transfection reagent (PolyPlus) or HEK293 cells were transfected with increasing quantity (0-4.8 pmoles) of hOct4 mRNA with the engineered 5′ and 3′ UTRs (SEQ ID NO: 1 and SEQ ID NO: 34, respectively) (referred to as RF5′3′ herein) using EXPIfectamine transfection reagent. Twenty-four hours post-transfection cells were fixed, permeabilized and stained with conjugated antibodies to detect Oct4, Sox2, Lin28 or Nanog protein expression via flow cytometry, as shown.

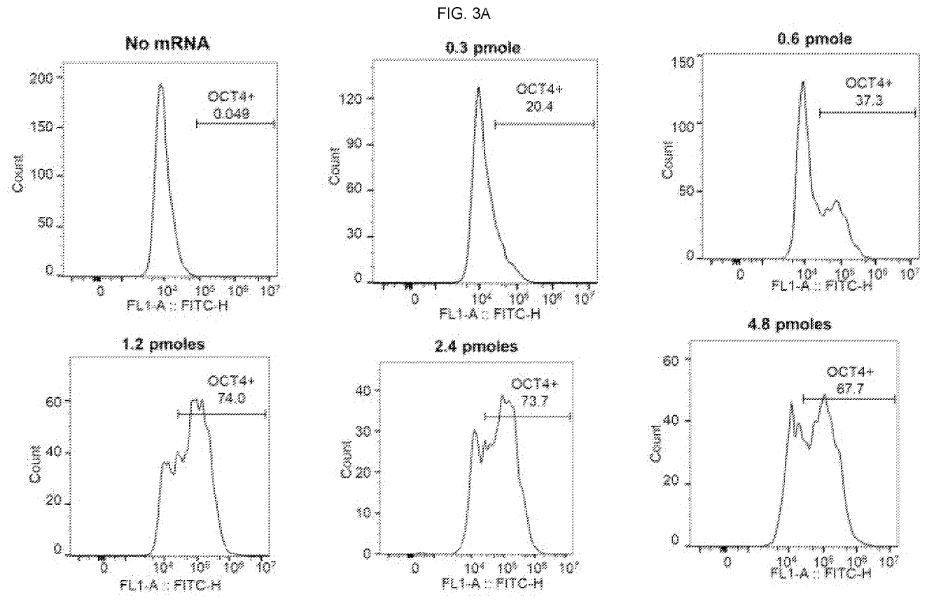

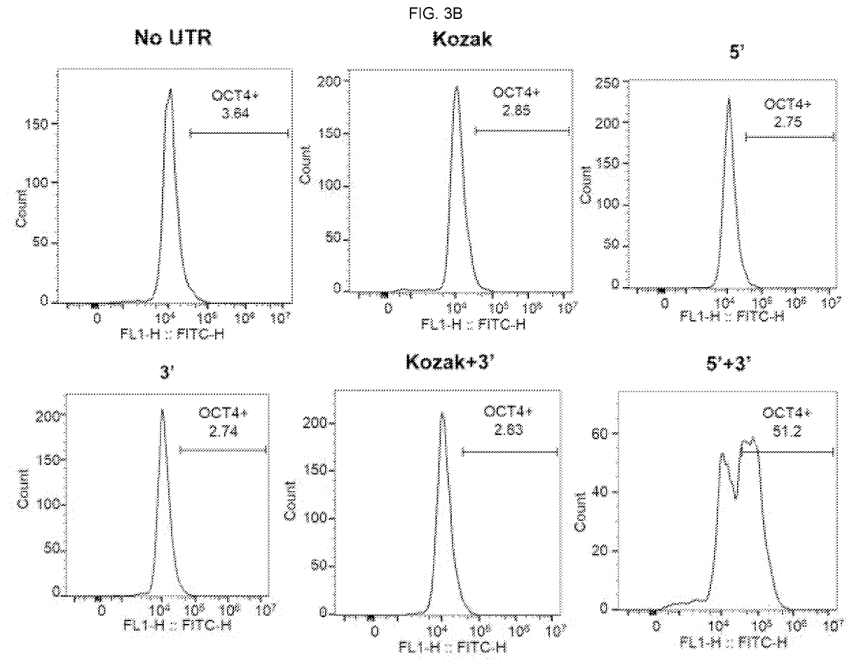

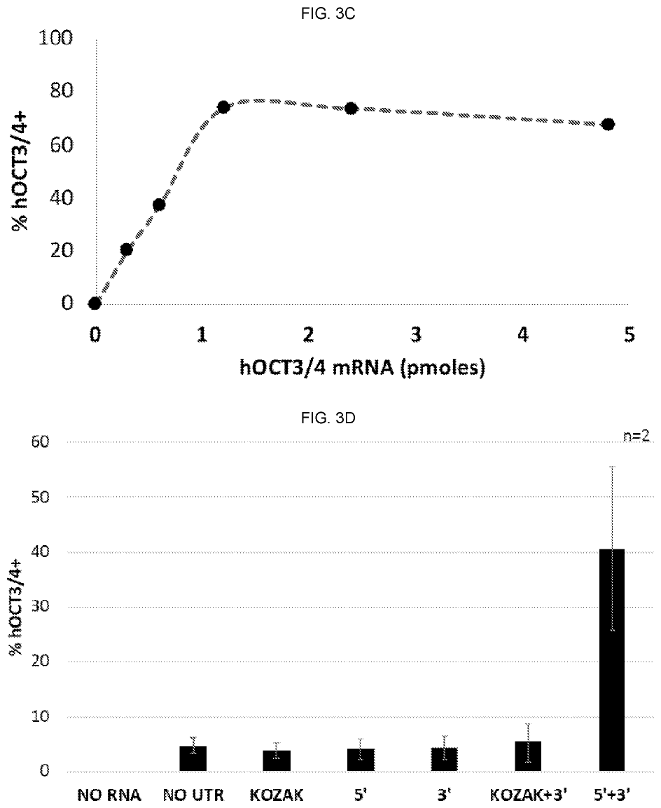

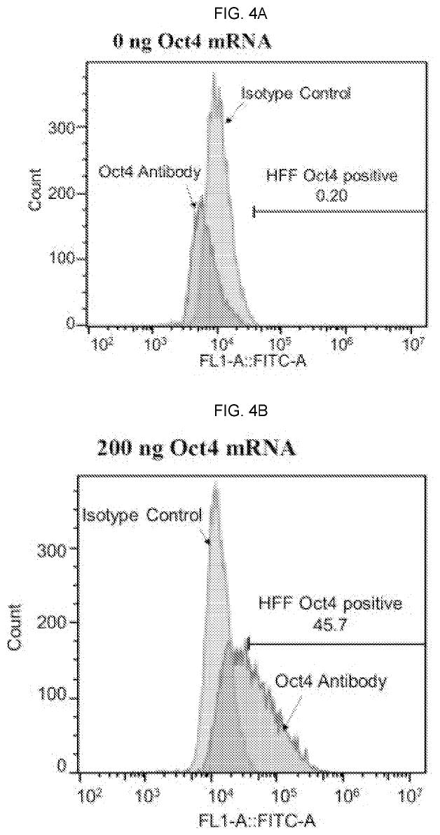

As shown in FIGS. 3A and 3C, the hOct4 mRNA with the RF5′3′ resulted in elevated levels of its protein within HEK293 cells after 24 hours (FIGS. 3A and 3C). The percentage of hOct4+ cells reached the maximum (75%) at 1.2 pmoles of mRNA per 2.0×10⁵ cells. By transfecting HEK293 cells with equimolar hOct4 transcripts with no UTR, Kozak only, 5′UTR only, 3′UTR only, Kozak and 3′ UTR, or the RF5′3′ (SEQ ID NO: 84), only those with RF5′3′ displayed significant percentage of hOct4+ cells (40%) (FIGS. 3B and 3D). Further, the median {number of experiments=2} hOct4 intensity was 4-fold higher in those cells treated with hOct4 mRNA with RF5′3′ (FIG. 3D).

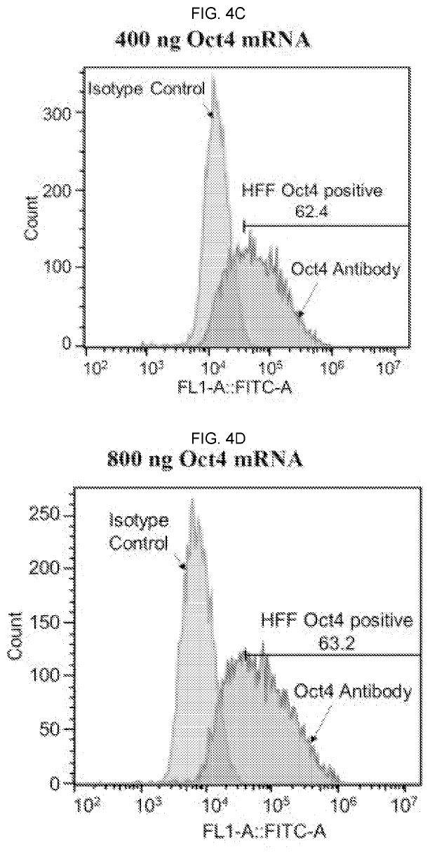

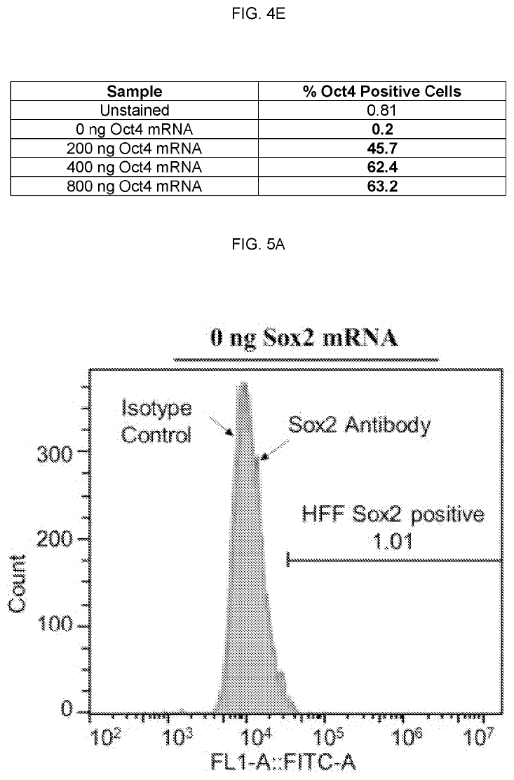

Further, as shown in FIGS. 4A-4E, transfection of fibroblasts with low amounts of Oct4 mRNA with the RF5′3′ (200 ng) increased Oct4 protein expression above baseline Oct4 expression in 45.7% of fibroblasts. Further increasing the amount of Oct4 mRNA with the RF5′3′ delivered to the fibroblasts to 400 ng and 800 ng resulted in higher percentage of cells expressing the RF (62.4% and 63.2% respectively) (FIG. 4E).

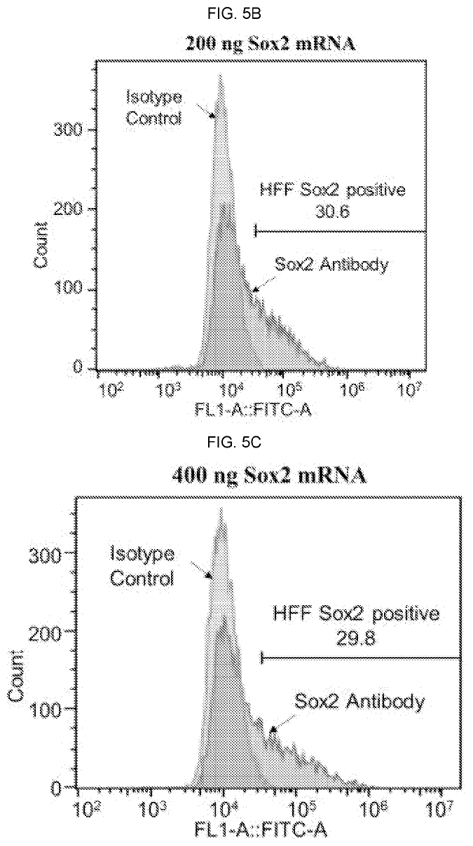

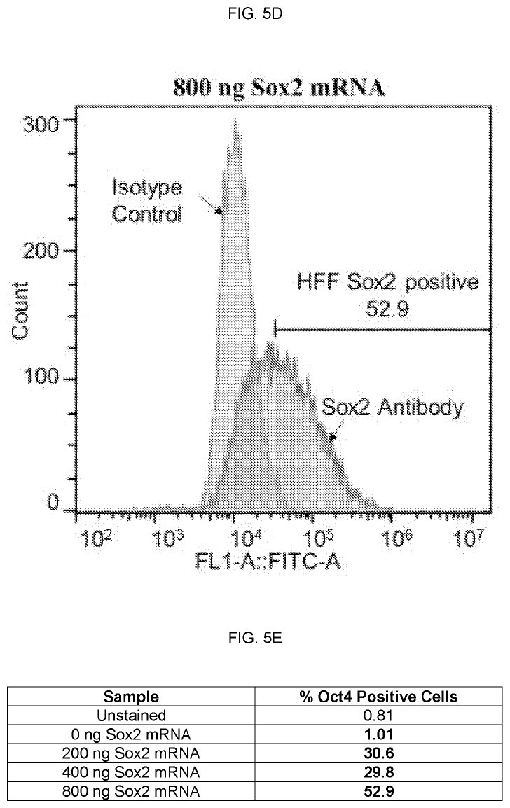

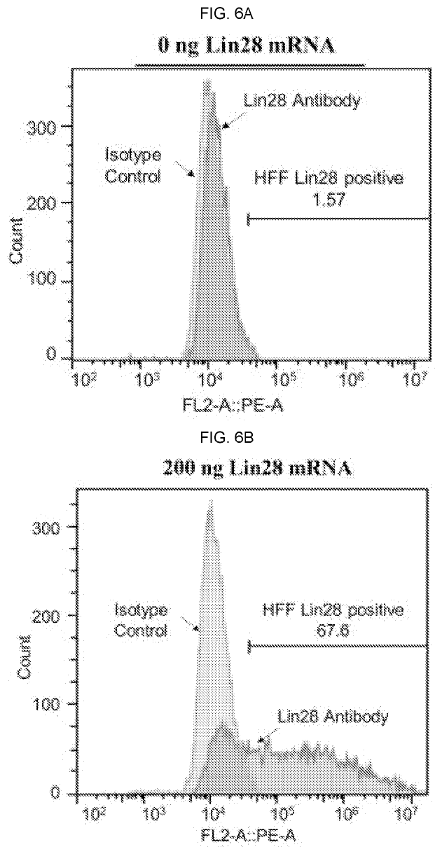

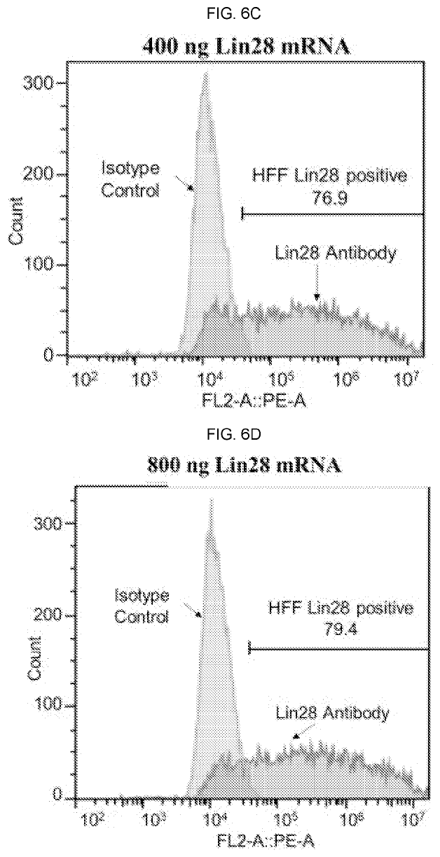

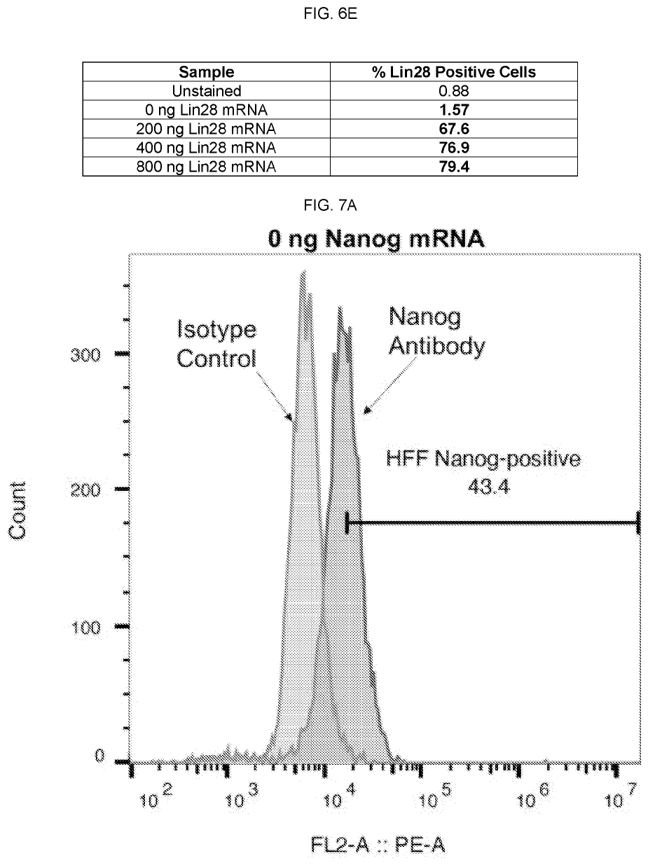

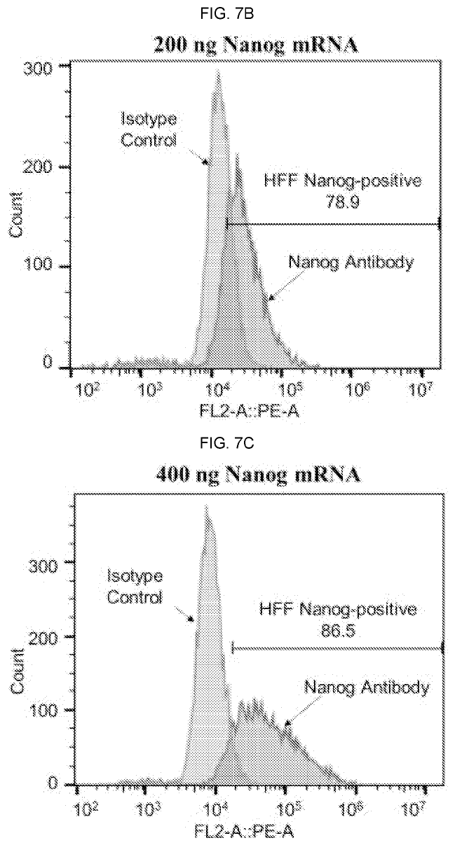

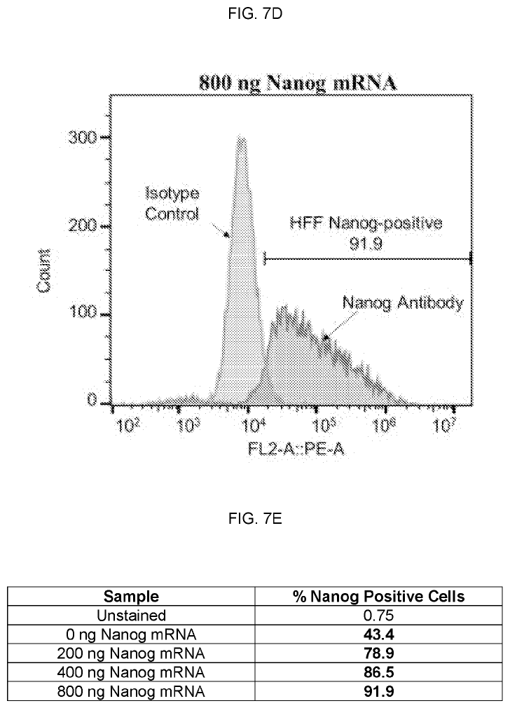

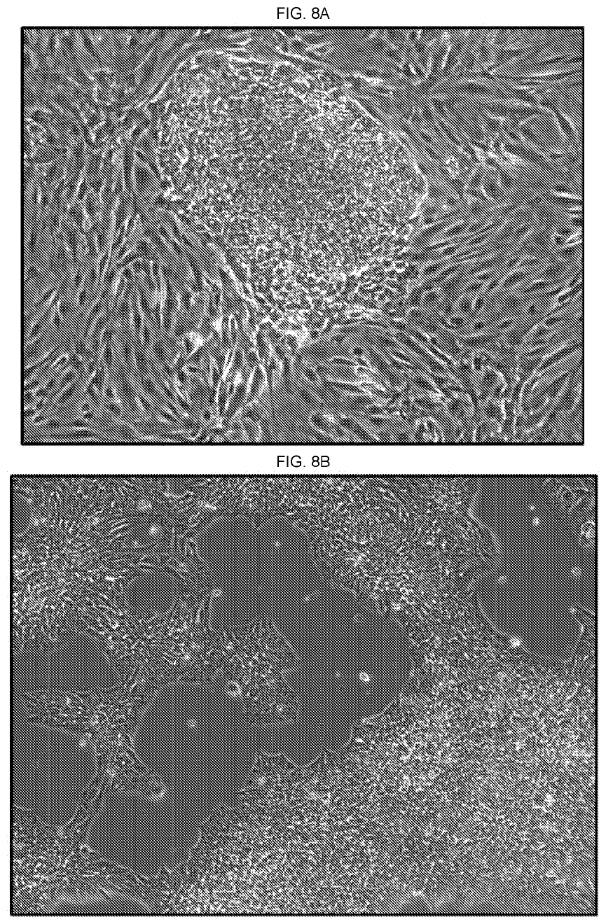

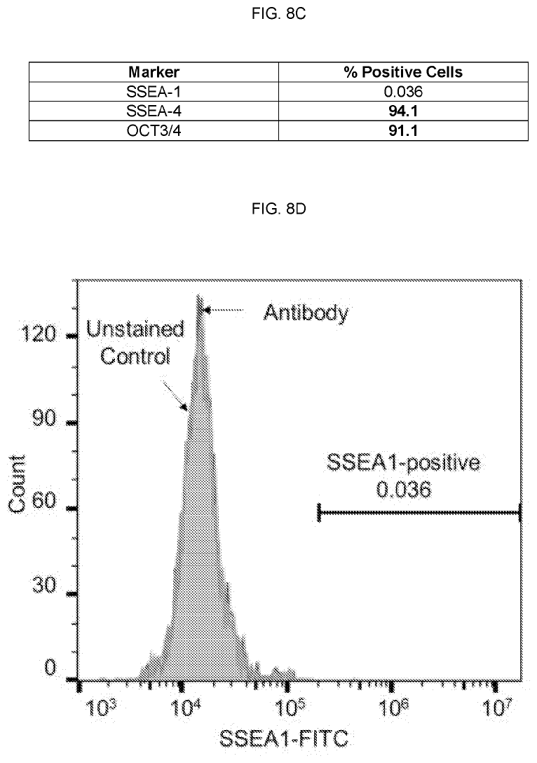

Similarly, as shown in FIGS. 5A-5E, transfection of human foreskin fibroblasts with synthetic Sox2 mRNA containing the RF5′3′ increased Sox2 protein expression in a dose dependent manner (30.6% positive cells for 200 ng mRNA; 52.9% positive cells for 800 ng) (FIG. 5E). Transfection with both Lin28 and Nanog synthetic mRNA containing the RF5′3′ resulted in higher percentage of cells expressing the Lin28 and Nanog proteins even at low quantities of mRNA delivered to fibroblasts (67.6% Lin28-positive cells and 78.9% Nanog-positive cells) (FIGS. 6A-6E and FIGS. 7A-7E, respectively).

These experiments showed that transfecting human foreskin fibroblasts and HEK293 cells with synthetic mRNA with the RF5′3′ prompted significant expression of RF essential for reprogramming of somatic cells to induced pluripotent stem cells.

Example 4. Reprogramming of human foreskin fibroblasts to induced pluripotent stem cells (iPSC) using synthetic mRNA RF containing the engineered 5′ and 3′ UTRs (SEQ ID NO: 1 and SEQ ID NO: 34, respectively) (referred to as RF5′3′ herein).

To test the ability of synthetic mRNA to reprogram somatic cells to iPSC early passage human foreskin fibroblasts were transfected with RF Oct4, Sox2, Klf4, cMyc, Nanog, Lin28 mRNAs, each containing the RF5′3′. To accomplish this, human foreskin fibroblasts were plated into 6-well plates, 20,000 cells/well, and were transfected every 24 hours over 4 consecutive days with Lipofectamine™ RNAiMAX (ThermoFisher Scientific) and 800 ng Reprogramming Cocktail containing 800 ng equimolar quantities of RF Oct4, Sox2, Klf4, cMyc, Nanog, Lin28 synthetic mRNA containing the RF5′3′, 600 ng IEF B18R, E3, K3, non-engineered synthetic mRNA, and 400 ng miRNAs.

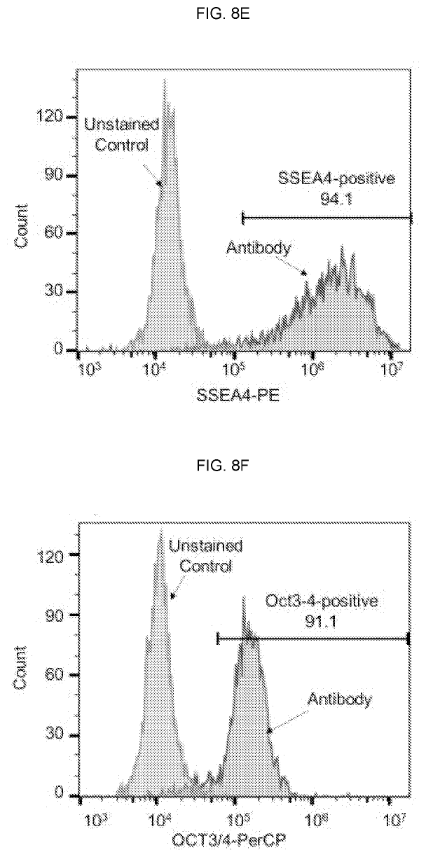

Colonies of reprogrammed fibroblasts were clearly visible 14 days after completion of the transfection cycle (FIG. 8A). Multiple colonies were isolated and expanded by passaging in NutriStem™ hPSC XF Culture Medium on iMatrix-511 substrate. Transfected cells display the typical morphology of pluripotent stem cells, small-sized, with enlarged nucleus and prominent nucleoli, and formed colonies with clearly defined borders (FIG. 8B). Flow cytometry analysis of pluripotent markers expression showed high levels of expression for OCT4 and SSEA-4 (91.1% OCT4-positive cells and 94.1% SSEA-4-positive cells), and lack of expression for the SSEA-1 differentiation marker.

Together, these data support the ability of synthetic mRNA with the RF5′3′ to reprogram somatic cells to iPSC.

Example 5. Reprogramming of human adult dermal fibroblasts to induced pluripotent stem cells (iPSC) using synthetic mRNA Reprogramming Factors containing the engineered 5′ and 3′ UTRs (SEQ ID NO: 1 and SEQ ID NO: 34, respectively) (referred to as RF5′3′ herein).

To further test the ability of synthetic mRNA to reprogram somatic cells to iPSC early passage adult dermal fibroblasts were transfected with RF Oct4, Sox2, Klf4, cMyc, Nanog, Lin28 mRNAs, each containing the (RF5′3′. To accomplish this, adult dermal fibroblasts were plated into 6-well plates, 30,000 cells/well, and were transfected every 24 hours over 4 consecutive days with Lipofectamine™ RNAiMAX (ThermoFisher Scientific) and 800 ng Reprogramming Cocktail containing equimolar quantities of RF Oct4, Sox2, Klf4, cMyc, Nanog, Lin28 synthetic mRNA containing the RF5′3′, 600 ng IEF B18R, E3, K3 non-engineered synthetic mRNA and 400 ng miRNAs.

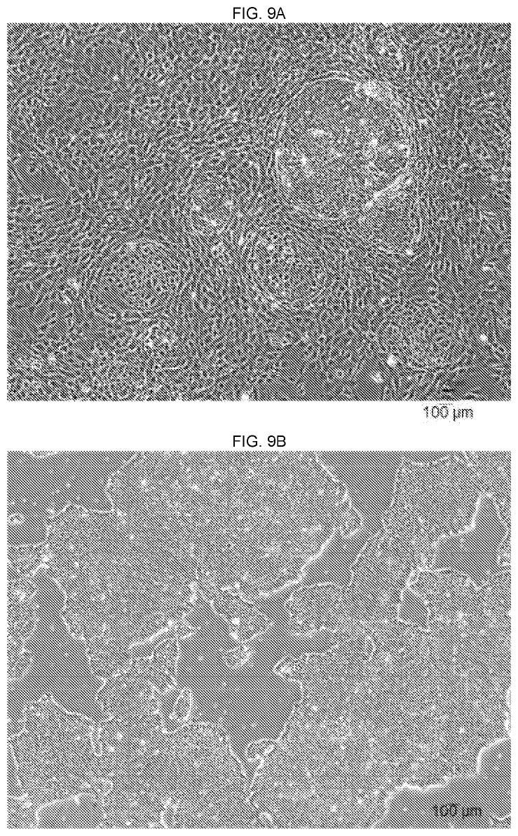

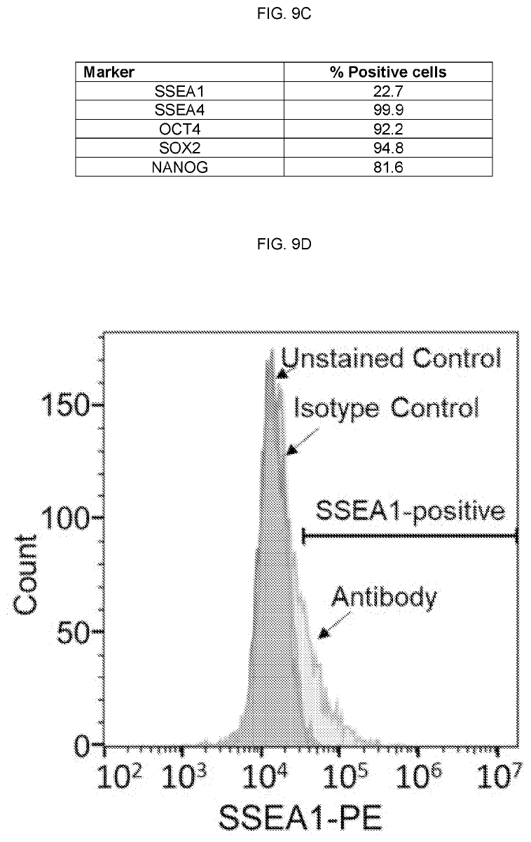

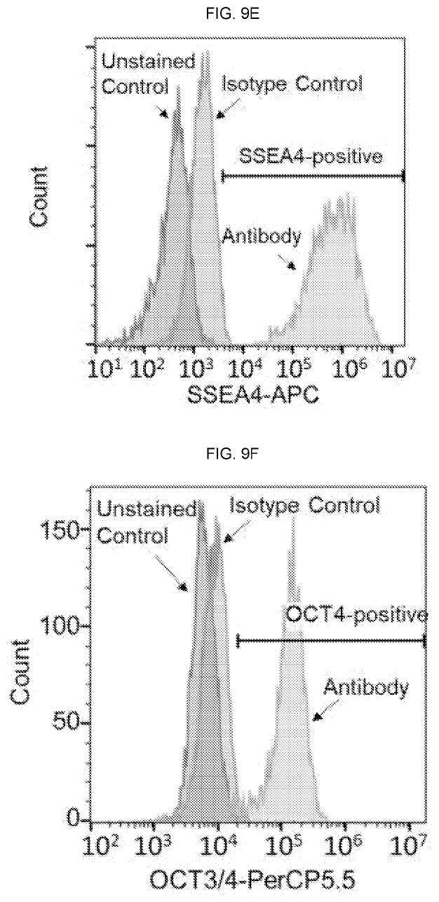

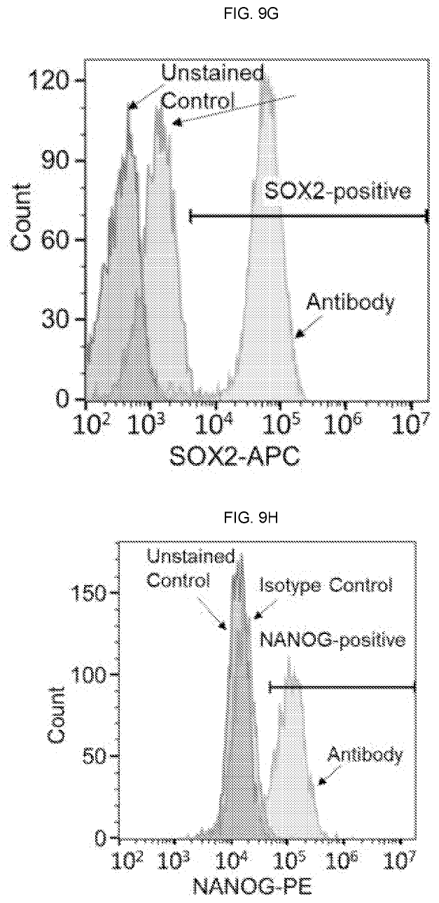

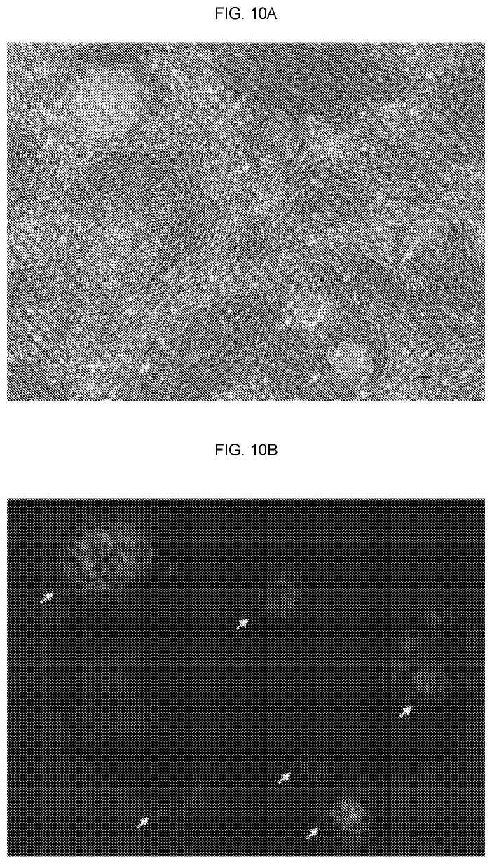

Colonies of reprogrammed fibroblasts were clearly visible 7-14 days after completion of the transfection cycle (FIG. 9A). Multiple colonies were isolated by bulk-passaging and expanded in NutriStem™ hPSC XF Culture Medium on iMatrix-511 substrate for five passages. Transfected cells display the typical morphology of pluripotent stem cells, small-sized, with enlarged nucleus and prominent nucleoli, and formed colonies with clearly defined borders (FIG. 9B). Flow cytometry analysis of pluripotent markers expression by adult dermal fibroblast-derived iPSC showed high levels of expression for OCT4, SOX2, NANOG and SSEA-4 (92.2%, OCT4-positive cells, 94.8% SOX2-positive cells, 81.6% NANOG-positive cells and 99.9% SSEA-4-positive cells, FIGS. 9C and 9E-9H) and lack of expression for the SSEA-1 differentiation marker (FIGS. 9C, 9D).

Together, these data support the ability of synthetic mRNA with the RF5′3′ to reprogram somatic cells to iPSC.

Example 6. Reprogramming of human adult dermal fibroblasts to induced pluripotent stem cells (iPSC) using synthetic mRNA Reprogramming Factors and IEF containing the engineered 5′ and 3′ UTRs.

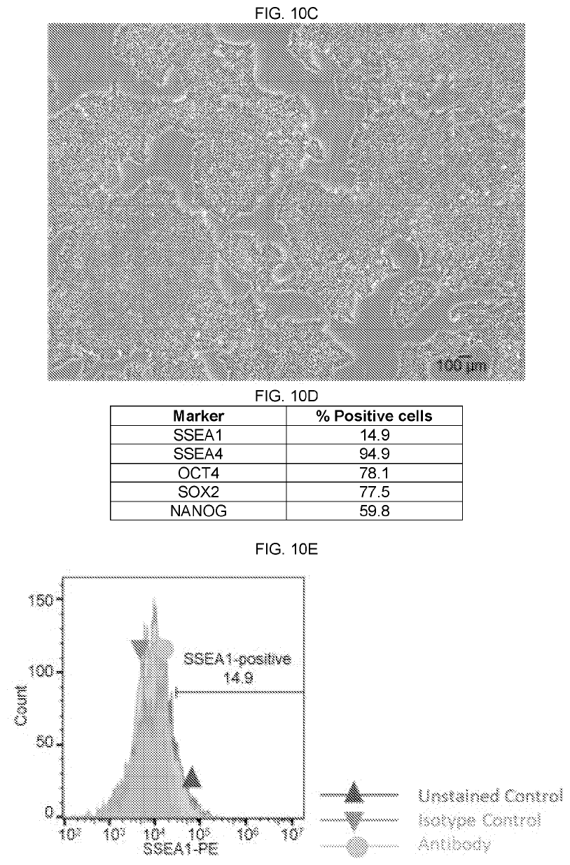

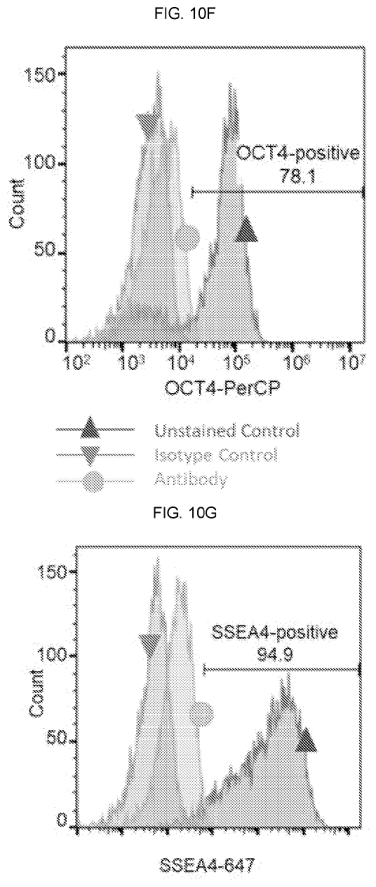

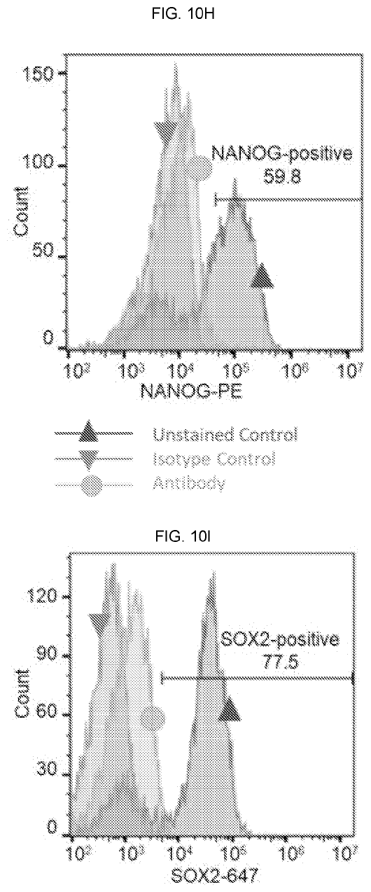

To further demonstrate the ability of the engineered synthetic mRNA to successfully reprogram somatic cells to iPSC, the reprogramming experiment was performed using engineered mRNA for both RF and IEF. To this purpose, adult dermal fibroblast cells were transfected with 800 ng Reprogramming Cocktail containing engineered synthetic RF mRNA (Oct4, Sox2, Klf4, cMyc, Nanog, Lin28), 600 ng engineered IEF mRNA (B18R, E3, K3), and 400 ng miRNAs. Multiple colonies of reprogrammed fibroblasts were clearly visible by day 10 (FIG. 10A, arrows) and all colonies were positive for the TRA-1-60 stem cell surface marker (FIG. 10B). Bulk-passaging and expansion of colonies for three passages generated iPSC with typical morphology of pluripotent stem cells (FIG. 10C), which showed high levels of expression for pluripotency markers (78.1%, OCT4-positive cells, 77.5% SOX2-positive cells, 59.8% NANOG-positive cells and 94.9% SSEA-4-positive cells, FIGS. 10D-101)) and low levels of expression for differentiation markers (FIGS. 10D, 10E).

Together, these data support the ability of synthetic mRNA with the RF5′3′ to reprogram somatic cells to iPSC.

Example 7. Reprogramming of human foreskin fibroblasts and adult dermal fibroblasts to induced pluripotent stem cells (iPSC) using synthetic mRNA Reprogramming Factors containing the engineered 5′ and 3′ UTRs and SV40 large T antigen (SV40Tag).

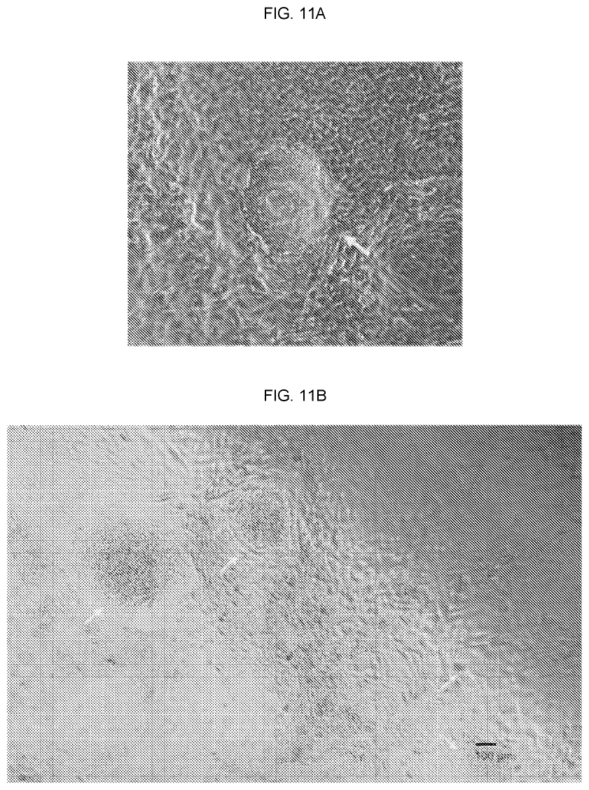

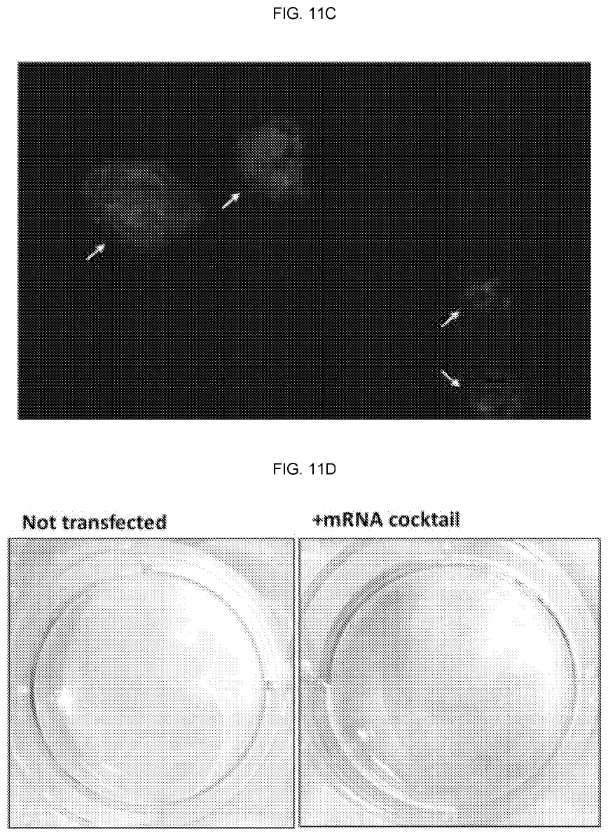

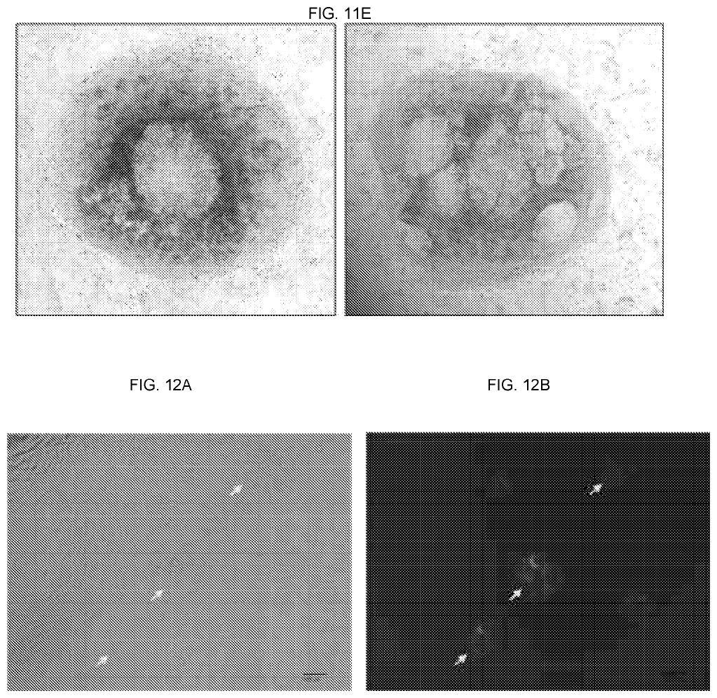

To further demonstrate the ability of the engineered mRNA to successfully reprogram somatic cells to iPSC, the reprogramming experiment was performed using engineered mRNA RF including Oct4, Sox2, Klf4, cMyc, Nanog, Lin28 and SV40Tag. To this purpose, adult dermal fibroblast and human foreskin fibroblast cells were transfected with 1000 ng Reprogramming Cocktail containing engineered RF mRNA (Oct4, Sox2, Klf4, cMyc, Nanog, Lin28, SV40Tag), 600 ng non-engineered IEF mRNA (B18R, E3, K3) and 400 ng miRNAs. Transfection of adult dermal fibroblasts with the SV40Tag-containing Reprogramming Cocktail resulted in multiple colonies of reprogrammed fibroblasts (FIGS. 11A-11B) which were positive for the TRA-1-60 stem cell surface marker (FIG. 11C). Transfection of human foreskin fibroblasts with the SV40Tag-containing Reprogramming Cocktail resulted in several iPSC colonies as demonstrated by the positive staining for Alkaline Phosphatase activity, a biomarker of stem cells (FIGS. 11D-11E).

Together, these data support the ability of synthetic mRNA with the RF5′3′ to reprogram somatic cells to iPSC.

Example 8. Reprogramming of human adult dermal fibroblasts to induced pluripotent stem cells (iPSC) using synthetic mRNA containing the engineered 5′ and 3′ UTRs and B18R purified protein.

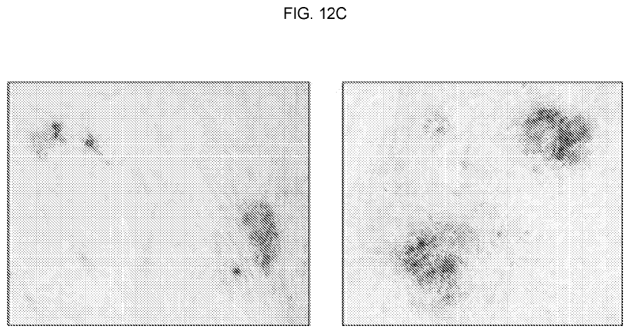

To further demonstrate the ability of the engineered mRNA to successfully reprogram somatic cells to iPSC, the reprogramming experiment was performed by transfecting engineered mRNA RF in the presence of purified B18R protein in place of transfection with engineered or non-engineered IEF mRNA. To this purpose, adult dermal fibroblasts were transfected with Reprogramming Cocktail containing 800 ng engineered RF mRNA (Oct4, Sox2, Klf4, cMyc, Nanog, Lin28) and 400 ng miRNAs, delivered to cells via lipofection in medium supplemented with 200 ng/mL B18R protein. Transfection of adult dermal fibroblasts with the Reprogramming Cocktail generated multiple colonies by Day 14 (FIG. 12A) and the colonies were positive for the TRA-1-60 stem cell marker (FIG. 12B) and stem cell-specific Alkaline Phosphatase activity (FIG. 12C).

Together, these data support the ability of synthetic mRNA with the RF5′3′ to reprogram somatic cells to iPSC.

Example 9. Reprogramming of human foreskin and/or adult fibroblasts to induced pluripotent stem cells (iPSC) using synthetic mRNA containing the engineered 5′ and 3′ UTRs.

According to certain embodiments, to test the ability of synthetic mRNA to reprogram somatic cells to iPSC early passage human foreskin fibroblasts and/or human adult fibroblasts are transfected with RF Oct4, Sox2, Klf4, cMyc, Nanog, Lin28 mRNAs, each containing the engineered 5′ and 3′ UTRs (“RF5′3′”). To accomplish this, prior to plating the fibroblasts, the cells are electroporated (using Neon Electroporation system (ThermoFisher Scientific)) using mRNA of IEF (E3, K3, and B18R) containing the engineered 5′ and 3′ UTRs and then are plated into 6-well plates, 50,000 cells/well. After six hours, allowing the cells to attach to the substrate, the resulting cells are transfected every 24 hours over 4 consecutive days with Lipofectamine™ RNAiMAX (ThermoFisher Scientific) and 800 ng Reprogramming cocktail containing equimolar quantities of Oct4, Sox2, Klf4, cMyc, Nanog, Lin28 synthetic mRNA containing the engineered 5′ and 3′ UTRs.

According to certain embodiments, to test the ability of synthetic mRNA to reprogram somatic cells to iPSC early passage human foreskin fibroblasts (“HFF”) and/or adult dermal fibroblasts are transfected with RF Oct4, Sox2, Klf4, cMyc, Nanog, Lin28 mRNAs, each containing the engineered 5′ and 3′ UTRs (“RF5′3′”). To accomplish this, cells are plated into 6-well plates, 20,000 cells/well, and are transfected using with mRNA of IEF (E3, K3, and B18R) containing the engineered 5′ and 3′ UTRs with Lipofectamine™ RNAiMAX (ThermoFisher Scientific). Six hours later, and after the cells have recovered and are expressing the IEF, the cells are transfected every 24 hours over 4 consecutive days with Lipofectamine™ RNAiMAX (ThermoFisher Scientific) and 800 ng Reprogramming cocktail containing equimolar quantities of Oct4, Sox2, Klf4, cMyc, Nanog, Lin28 synthetic mRNA containing the engineered 5′ and 3′ UTRs.

Colonies of reprogrammed fibroblasts are clearly visible 14 days after completion of the transfection cycle and were positive for the TRA-1-60 stem cell marker.

Together, these data support the ability of synthetic mRNA to reprogram somatic cells to iPSC using the RF5′3′.

Example 10. Reprogramming of Human Mesenchymal Stromal Cells (“hMSC”) to induced pluripotent stem cells (iPSC) using synthetic mRNA containing the engineered 5′ and 3′ UTRs.

To test the ability of synthetic mRNA to reprogram a variety of somatic cells to iPSC, hMSCs will be transfected with RF Oct4, Sox2, Klf4, cMyc, Nanog, and Lin28 mRNAs, each containing the engineered 5′ and 3′ UTRs (RF5′3′). To accomplish this, hMSC cells will be plated into 6-well plates, and will be transfected every 24 hours over 4 consecutive days with Lipofectamine™ RNAiMAX (ThermoFisher Scientific) and 800 ng Reprogramming cocktail containing equimolar quantities of Oct4, Sox2, Klf4, cMyc, Nanog, and Lin28 synthetic mRNA containing the engineered 5′ and 3′ UTRs.

According to certain embodiments, the IEF will also be utilized to increase production of iPSC. To accomplish this, prior to plating the hMSCs, the cells will be electroporated (using Neon Electroporation system (ThermoFisher Scientific)) using mRNA of IEF (E3, K3, and B18R) containing the engineered 5′ and 3′ UTRs and then will be plated into 6-well plates, 20,000 cells/well. After six hours, allowing the cells to attach to the substrate, the resulting cells will be transfected every 24 hours over 4 consecutive days with Lipofectamine™ RNAiMAX (ThermoFisher Scientific) and 800 ng Reprogramming cocktail containing equimolar quantities of Oct4, Sox2, Klf4, cMyc, Nanog, and Lin28 synthetic mRNA containing the engineered 5′ and 3′ UTRs.

According to alternative embodiments, cells will be plated into 6-well plates, 20,000 cells/well, and will be transfected using with mRNA of IEF (E3, K3, and B18R) containing the engineered 5′ and 3′ UTRs with Lipofectamine™ RNAiMAX (ThermoFisher Scientific). Six hours later, and after the cells have recovered and are expressing the IEF, the cells will be transfected every 24 hours over 4 consecutive days with Lipofectamine™ RNAiMAX (ThermoFisher Scientific) and 800 ng Reprogramming cocktail containing equimolar quantities of Oct4, Sox2, Klf4, cMyc, Nanog, and Lin28 synthetic mRNA containing the engineered 5′ and 3′ UTRs.

According to alternative embodiments, cells will be plated into 6-well plates, 20,000 cells/well, in media supplemented with 200 ng/ml B18R protein. Then the cells will be transfected every 24 hours over 4 consecutive days with Lipofectamine™ RNAiMAX (ThermoFisher Scientific) and 800 ng Reprogramming cocktail containing equimolar quantities of Oct4, Sox2, Klf4, cMyc, Nanog, and Lin28 synthetic mRNA containing the engineered 5′ and 3′ UTRs.

Colonies of reprogrammed cells will be clearly visible 14 days after completion of the transfection cycle.

Together, these data support the ability of synthetic mRNA to reprogram a wide variety of somatic cells to iPSC using the RF5′3′.

Example 11. Reprogramming of human CD34+ cells to induced pluripotent stem cells (iPSC) using synthetic mRNA containing the engineered 5′ and 3′ UTRs.

According to certain embodiments, to test the ability of synthetic mRNA to reprogram somatic cells to iPSC human umbilical cord blood, CD34+ hematopoietic progenitor cells (HPC) (obtained from Bloodworks Northwest, Seattle, WA) and/or human mobilized peripheral blood CD34+ HPCs (obtained from Fred Hutch CCEH Core) will be transfected with RF Oct4, Sox2, Klf4, cMyc, Nanog, Lin28 mRNAs, each containing the engineered 5′ and 3′ UTRs (RF5′3′). To accomplish this, >100,000 CD34+ cells will undergo daily electroporations every 24 hours with 200-1000 ng of reprogramming cocktail containing equimolar quantities of Oct4, Sox2, Klf4, cMyc, Nanog, Lin28 and/or SV40 large T antigen (SV40Tag) synthetic mRNA containing the engineered 5′ and 3′ UTRs for consecutive days, using optimized parameters on the Neon Electroporation system (ThermoFisher Scientific, Waltham, MA).

According to certain embodiments, the IEF will also be utilized to increase production of iPSC. To accomplish this, the mRNA of IEF (E3, K3, and B18R) containing the engineered 5′ and 3′ UTRs will be added to the reprogramming cocktail mRNA and electroporated, or B18R protein will be supplemented to the CD34+ cell media at 200 ng/ml.

Colonies of reprogrammed cells will be clearly visible 14-21 days after completion of the electroporation cycle. Multiple colonies will be isolated and expanded by passaging in NutriStem™ hPSC XF Culture Medium on iMatrix-511 substrate. Transfected cells will display the typical morphology of pluripotent stem cells, small-sized, with enlarged nucleus and prominent nucleoli, and forming colonies with clearly defined borders. Flow cytometry analysis of pluripotent markers expression will show high levels of expression for OCT4 and SSEA-4 (91.1% OCT4-positive cells and 94.1% SSEA-4-positive cells), and will show a lack of expression for the SSEA-1 differentiation marker.

Example 12. Reprogramming of human adult dermal fibroblast to induced pluripotent stem cells (iPSC) using synthetic mRNA containing the engineered 5′ and 3′ UTRs without cMyc.

To test the ability of synthetic mRNA to reprogram somatic cells to iPSC without cMyc, human adult dermal fibroblasts (hAFs) are transfected with RF Oct4, Sox2, Klf4, Nanog, Lin28, and SV40 large T antigen (SV40Tag) mRNAs, each containing the engineered 5′ and 3′ UTRs (RF5′3′). To accomplish this, hAFs are plated into 6-well plates, 30,000 cells/well, and are transfected every 24 hours over 4 consecutive days with Lipofectamine™ RNAiMAX (ThermoFisher Scientific) and 800 ng reprogramming cocktail containing equimolar quantities of Oct4, Sox2, Klf4, Nanog, Lin28 and/or SV40Tag synthetic mRNA containing the engineered 5′ and 3′ UTRs.

According to certain embodiments, the IEF were also utilized to increase production of iPSC. To accomplish this, prior to plating the hAFs, the cells are electroporated (using Neon Electroporation system (ThermoFisher Scientific)) using mRNA of IEF (E3, K3, and B18R) containing the engineered 5′ and 3′ UTRs and then are plated into 6-well plates, 30,000 cells/well. After six hours, allowing the cells to attach to the substrate, the resulting cells are transfected every 24 hours over 4 consecutive days with Lipofectamine™ RNAiMAX (ThermoFisher Scientific) and 800 ng Reprogramming cocktail containing equimolar quantities of Oct4, Sox2, Klf4, Nanog, Lin28 and/or SV40Tag synthetic mRNA containing the engineered 5′ and 3′ UTRs.

According to alternative embodiments, cells are plated into 6-well plates, 30,000 cells/well, and are transfected using with mRNA of IEF (E3, K3, and B18R) containing the engineered 5′ and 3′ UTRs with Lipofectamine™ RNAiMAX (ThermoFisher Scientific). Six hours later, and after the cells have recovered and are expressing the IEF, the cells are transfected every 24 hours over 4 consecutive days with Lipofectamine™ RNAiMAX (ThermoFisher Scientific) and 800 ng Reprogramming cocktail containing equimolar quantities of Oct4, Sox2, Klf4, Nanog, Lin28 and SV40Tag synthetic mRNA containing the engineered 5′ and 3′ UTRs.

Colonies of reprogrammed cells are clearly visible 14 days after completion of the transfection cycle.

Together, these data support the ability of synthetic mRNA to reprogram somatic cells to iPSC using the RF5′3′.

Example 13. Reprogramming of human adult dermal fibroblast to induced pluripotent stem cells (iPSC) using synthetic mRNA containing the engineered 5′ and 3′ UTRs was not dependent on one specific transfection schedule.

To test the ability of synthetic mRNA to reprogram somatic cells to iPSC using a variety of transfection schedules, human adult dermal fibroblasts (hAFs) are transfected with RF Oct4, Sox2, Klf4, Nanog, Lin28, and c-Myc mRNAs, each containing the engineered 5′ and 3′ UTRs (RF5′3′). To accomplish this, hAFs are plated into 6-well plates, 20,000 cells/well, and are transfected 1) every 24 hours over 3 consecutive days, 2) every 30 hours over 4 consecutive days, 3) every 24 hours over 5 days, 4) every 30 hours over 5 days, or 5) every 48 over 6 days with Lipofectamine™ RNAiMAX (ThermoFisher Scientific) and 800 ng reprogramming cocktail containing equimolar quantities of Oct4, Sox2, Klf4, Nanog, Lin28 and c-Myc synthetic mRNA containing the engineered 5′ and 3′ UTRs.

According to certain embodiments, the IEF are also utilized to increase production of iPSC. To accomplish this, prior to plating the adult dermal fibroblasts, the cells are electroporated (using Neon Electroporation system (ThermoFisher Scientific)) using mRNA of IEF (E3, K3, and B18R) containing the engineered 5′ and 3′ UTRs and then are plated into 6-well plates, 20,000 cells/well. After six hours, allowing the cells to attach to the substrate, the resulting cells are transfected according to the schedule described above with 800 ng reprogramming cocktail containing equimolar quantities of Oct4, Sox2, Klf4, Nanog, Lin28 and c-Myc synthetic mRNA containing the engineered 5′ and 3′ UTRs.

According to certain embodiments, B18R protein is also utilized to increase production of iPSC. To accomplish this, after plating the hAFs into 6-well plates, 20,000 cells/well, the cells are incubated with the B18R protein. After six hours, allowing the cells to attach to the substrate, the resulting cells are transfected according to the schedule described above with 800 ng reprogramming cocktail containing equimolar quantities of Oct4, Sox2, Klf4, Nanog, Lin28 and c-Myc synthetic mRNA containing the engineered 5′ and 3′ UTRs.

According to alternative embodiments, cells are plated into 6-well plates, 20,000 cells/well, and are transfected using mRNA of IEF (E3, K3, and B18R) containing the engineered 5′ and 3′ UTRs with Lipofectamine™ RNAiMAX (ThermoFisher Scientific). Six hours later, and after the cells have recovered and are expressing the IEF, the cells are transfected according to the transfection schedule described above 800 ng reprogramming cocktail containing equimolar quantities of Oct4, Sox2, Klf4, Nanog, Lin28 and c-Myc synthetic mRNA containing the engineered 5′ and 3′ UTRs.

Colonies of reprogrammed cells are clearly visible 14 days after completion of the transfection cycle.

Together, these data support the ability of synthetic mRNA to reprogram somatic cells to iPSC using the RF5′3′ and using a variety of transfection schedules.

### (xv) CLOSING PARAGRAPHS

The nucleic acid and amino acid sequences provided herein are shown using letter abbreviations for nucleotide bases and amino acid residues, as defined in 37 C.F.R. § 1.831-1.835 and set forth in WIPO Standard ST.26 (implemented on Jul. 1, 2022). Only one strand of each nucleic acid sequence is shown, but the complementary strand is understood as included in embodiments where it would be appropriate.

Variants of the sequences disclosed and referenced herein are also included. Guidance in determining which amino acid residues can be substituted, inserted, or deleted without abolishing biological activity can be found using computer programs well known in the art, such as DNASTAR™ (Madison, Wisconsin) software. Preferably, amino acid changes in the protein variants disclosed herein are conservative amino acid changes, i.e., substitutions of similarly charged or uncharged amino acids. A conservative amino acid change involves substitution of one of a family of amino acids which are related in their side chains.

In a peptide or protein, suitable conservative substitutions of amino acids are known to those of skill in this art and generally can be made without altering a biological activity of a resulting molecule. Those of skill in this art recognize that, in general, single amino acid substitutions in non-essential regions of a polypeptide do not substantially alter biological activity (see, e.g., Watson et al. Molecular Biology of the Gene, 4th Edition, 1987, The Benjamin/Cummings Pub. Co., p. 224). Naturally occurring amino acids are generally divided into conservative substitution families as follows: Group 1: Alanine (Ala), Glycine (Gly), Serine (Ser), and Threonine (Thr); Group 2: (acidic): Aspartic acid (Asp), and Glutamic acid (Glu); Group 3: (acidic; also classified as polar, negatively charged residues and their amides): Asparagine (Asn), Glutamine (Gln), Asp, and Glu; Group 4: Gln and Asn; Group 5: (basic; also classified as polar, positively charged residues): Arginine (Arg), Lysine (Lys), and Histidine (His); Group 6 (large aliphatic, nonpolar residues): Isoleucine (Ile), Leucine (Leu), Methionine (Met), Valine (Val) and Cysteine (Cys); Group 7 (uncharged polar): Tyrosine (Tyr), Gly, Asn, Gln, Cys, Ser, and Thr; Group 8 (large aromatic residues): Phenylalanine (Phe), Tryptophan (Trp), and Tyr; Group 9 (non-polar): Proline (Pro), Ala, Val, Leu, Ile, Phe, Met, and Trp; Group 11 (aliphatic): Gly, Ala, Val, Leu, and Ile; Group 10 (small aliphatic, nonpolar or slightly polar residues): Ala, Ser, Thr, Pro, and Gly; and Group 12 (sulfur-containing): Met and Cys. Additional information can be found in Creighton (1984) Proteins, W.H. Freeman and Company.

In making such changes, the hydropathic index of amino acids may be considered. The importance of the hydropathic amino acid index in conferring interactive biologic function on a protein is generally understood in the art (Kyte and Doolittle, 1982, J. Mol. Biol. 157 (1), 105-32). Each amino acid has been assigned a hydropathic index on the basis of its hydrophobicity and charge characteristics (Kyte and Doolittle, 1982). These values are: Ile (+4.5); Val (+4.2); Leu (+3.8); Phe (+2.8); Cys (+2.5); Met (+1.9); Ala (+1.8); Gly (−0.4); Thr (−0.7); Ser (−0.8); Trp (−0.9); Tyr (−1.3); Pro (−1.6); His (−3.2); Glutamate (−3.5); Gln (−3.5); aspartate (−3.5); Asn (−3.5); Lys (−3.9); and Arg (−4.5).

It is known in the art that certain amino acids may be substituted by other amino acids having a similar hydropathic index or score and still result in a protein with similar biological activity, i.e., still obtain a biological functionally equivalent protein. In making such changes, the substitution of amino acids whose hydropathic indices are within +2 is preferred, those within +1 are particularly preferred, and those within +0.5 are even more particularly preferred. It is also understood in the art that the substitution of like amino acids can be made effectively on the basis of hydrophilicity.

As detailed in U.S. Pat. No. 4,554,101, the following hydrophilicity values have been assigned to amino acid residues: Arg (+3.0); Lys (+3.0); aspartate (+3.0±1); glutamate (+3.0±1); Ser (+0.3); Asn (+0.2); Gln (+0.2); Gly (0); Thr (−0.4); Pro (−0.5±1); Ala (−0.5); His (−0.5); Cys (−1.0); Met (−1.3); Val (−1.5); Leu (−1.8); Ile (−1.8); Tyr (−2.3); Phe (−2.5); Trp (−3.4). It is understood that an amino acid can be substituted for another having a similar hydrophilicity value and still obtain a biologically equivalent, and in particular, an immunologically equivalent protein. In such changes, the substitution of amino acids whose hydrophilicity values are within ±2 is preferred, those within ±1 are particularly preferred, and those within ±0.5 are even more particularly preferred.

As outlined above, amino acid substitutions may be based on the relative similarity of the amino acid side-chain substituents, for example, their hydrophobicity, hydrophilicity, charge, size, and the like. As indicated elsewhere, variants of gene sequences can include codon optimized variants, sequence polymorphisms, splice variants, and/or mutations that do not affect the function of an encoded product to a statistically-significant degree.

Variants of the protein, nucleic acid, and gene sequences disclosed herein also include sequences with at least 70% sequence identity, 80% sequence identity, 85% sequence, 90% sequence identity, 95% sequence identity, 96% sequence identity, 97% sequence identity, 98% sequence identity, or 99% sequence identity to the protein, nucleic acid, or gene sequences disclosed herein.

“% sequence identity” refers to a relationship between two or more sequences, as determined by comparing the sequences. In the art, “identity” also means the degree of sequence relatedness between protein, nucleic acid, or gene sequences as determined by the match between strings of such sequences. “Identity” (often referred to as “similarity”) can be readily calculated by known methods, including those described in: Computational Molecular Biology (Lesk, A. M., ed.) Oxford University Press, NY (1988); Biocomputing: Informatics and Genome Projects (Smith, D. W., ed.) Academic Press, NY (1994); Computer Analysis of Sequence Data, Part I (Griffin, A. M., and Griffin, H. G., eds.) Humana Press, NJ (1994); Sequence Analysis in Molecular Biology (Von Heijne, G., ed.) Academic Press (1987); and Sequence Analysis Primer (Gribskov, M. and Devereux, J., eds.) Oxford University Press, NY (1992). Methods to determine identity are designed to give the best match between the sequences tested. Methods to determine identity and similarity are codified in publicly available computer programs. Sequence alignments and percent identity calculations may be performed using the Megalign program of the LASERGENE bioinformatics computing suite (DNASTAR, Inc., Madison, Wisconsin). Multiple alignment of the sequences can also be performed using the Clustal method of alignment (Higgins and Sharp CABIOS, 5, 151-153 (1989) with default parameters (GAP PENALTY=10, GAP LENGTH PENALTY=10). Relevant programs also include the GCG suite of programs (Wisconsin Package Version 9.0, Genetics Computer Group (GCG), Madison, Wisconsin); BLASTP, BLASTN, BLASTX (Altschul, et al., J. Mol. Biol. 215:403-410 (1990); DNASTAR (DNASTAR, Inc., Madison, Wisconsin); and the FASTA program incorporating the Smith-Waterman algorithm (Pearson, Comput. Methods Genome Res., [Proc. Int. Symp.] (1994), Meeting Date 1992, 111-20. Editor(s): Suhai, Sandor. Publisher: Plenum, New York, N.Y., Within the context of this disclosure it will be understood that where sequence analysis software is used for analysis, the results of the analysis are based on the “default values” of the program referenced. As used herein “default values” will mean any set of values or parameters, which originally load with the software when first initialized.

Variants also include nucleic acid molecules that hybridize under stringent hybridization conditions to a sequence disclosed herein and provide the same function as the reference sequence. Exemplary stringent hybridization conditions include an overnight incubation at 42° C. in a solution including 50% formamide, 5×SSC (750 mM NaCl, 75 mM trisodium citrate), 50 mM sodium phosphate (pH 7.6), 5×Denhardt's solution, 10% dextran sulfate, and 20 μg/ml denatured, sheared salmon sperm DNA, followed by washing the filters in 0.1×SSC at 50° C. Changes in the stringency of hybridization and signal detection are primarily accomplished through the manipulation of formamide concentration (lower percentages of formamide result in lowered stringency); salt conditions, or temperature. For example, moderately high stringency conditions include an overnight incubation at 37° C. in a solution including 6×SSPE (20×SSPE=3M NaCl; 0.2M NaH2PO4; 0.02M EDTA, pH 7.4), 0.5% SDS, 30% formamide, 100 μg/ml salmon sperm blocking DNA; followed by washes at 50° C. with 1×SSPE, 0.1% SDS. In addition, to achieve even lower stringency, washes performed following stringent hybridization can be done at higher salt concentrations (e.g. 5×SSC). Variations in the above conditions may be accomplished through the inclusion and/or substitution of alternate blocking reagents used to suppress background in hybridization experiments. Typical blocking reagents include Denhardt's reagent, BLOTTO, heparin, denatured salmon sperm DNA, and commercially available proprietary formulations. The inclusion of specific blocking reagents may require modification of the hybridization conditions described above, due to problems with compatibility.

“Specifically binds” refers to an association of a binding molecule to its cognate binding molecule with an affinity or Kₐ (i.e., an equilibrium association constant of a particular binding interaction with units of 1/M) equal to or greater than 10⁵ M⁻¹, while not significantly associating with any other molecules or components in a relevant environment sample. Binding molecules may be classified as “high affinity” or “low affinity”. In particular embodiments, “high affinity” binding molecules refer to those binding molecules with a Kₐ of at least 10⁷ M⁻¹, at least 10⁸ M⁻¹, at least 10⁹ M⁻¹, at least 10¹⁰ M⁻¹, at least 10¹¹ M⁻¹, at least 10¹² M⁻¹, or at least 10¹³ M⁻¹. In particular embodiments, “low affinity” binding molecules refer to those binding molecules with a Kₐ of up to 10⁷ M⁻¹, up to 10⁶ M⁻¹, up to 10⁵ M⁻¹. Alternatively, affinity may be defined as an equilibrium dissociation constant (Kₐ) of a particular binding interaction with units of M (e.g., 10⁻⁵ M to 10⁻¹³ M). In certain embodiments, a binding molecule may have “enhanced affinity,” which refers to a selected or engineered binding molecules with stronger binding to a cognate binding molecule than a wild type (or parent) binding molecule. For example, enhanced affinity may be due to a Kₐ (equilibrium association constant) for the cognate binding molecule that is higher than the reference binding molecule or due to a K_{d} (dissociation constant) for the cognate binding molecule that is less than that of the reference binding molecule, or due to an off-rate (K_{off}) for the cognate binding molecule that is less than that of the reference binding molecule. A variety of assays are known for detecting binding molecules that specifically bind a particular cognate binding molecule as well as determining binding affinities, such as Western blot, ELISA, and BIACORE® analysis (see also, e.g., Scatchard, et al., 1949, Ann. N.Y. Acad. Sci. 51:660; and U.S. Pat. Nos. 5,283,173, 5,468,614, or the equivalent).

Unless otherwise indicated, the practice of the present disclosure can employ conventional techniques of immunology, molecular biology, microbiology, cell biology and recombinant DNA. These methods are described in the following publications. See, e.g., Sambrook, et al. Molecular Cloning: A Laboratory Manual, 2nd Edition (1989); F. M. Ausubel, et al. eds., Current Protocols in Molecular Biology, (1987); the series Methods IN Enzymology (Academic Press, Inc.); M. MacPherson, et al., PCR: A Practical Approach, IRL Press at Oxford University Press (1991); MacPherson et al., eds. PCR 2: Practical Approach, (1995); Harlow and Lane, eds. Antibodies, A Laboratory Manual, (1988); and R. I. Freshney, ed. Animal Cell Culture (1987).

As will be understood by one of ordinary skill in the art, each embodiment disclosed herein can comprise, consist essentially of or consist of its particular stated element, step, ingredient or component. Thus, the terms “include” or “including” should be interpreted to recite: “comprise, consist of, or consist essentially of.” As used herein, the transition term “comprise” or “comprises” means has, but is not limited to, and allows for the inclusion of unspecified elements, steps, ingredients, or components, even in major amounts. The transitional phrase “consisting of” excludes any element, step, ingredient or component not specified. The transitional phrase “consisting essentially of” limits the scope of the embodiment to the specified elements, steps, ingredients or components and to those that do not materially affect the embodiment. As used herein, a material effect would cause a statistically significant reduction in RF expression observed with EEC described herein.

Unless otherwise indicated, all numbers expressing quantities of ingredients, properties such as molecular weight, reaction conditions, and so forth used in the specification and claims are to be understood as being modified in all instances by the term “about.” Accordingly, unless indicated to the contrary, the numerical parameters set forth in the specification and attached claims are approximations that may vary depending upon the desired properties sought to be obtained by the present invention. At the very least, and not as an attempt to limit the application of the doctrine of equivalents to the scope of the claims, each numerical parameter should at least be construed in light of the number of reported significant digits and by applying ordinary rounding techniques. When further clarity is required, the term “about” has the meaning reasonably ascribed to it by a person skilled in the art when used in conjunction with a stated numerical value or range, i.e. denoting somewhat more or somewhat less than the stated value or range, to within a range of ±20% of the stated value; ±19% of the stated value; ±18% of the stated value; ±17% of the stated value; ±16% of the stated value; ±15% of the stated value; ±14% of the stated value; ±13% of the stated value; ±12% of the stated value; ±11% of the stated value; ±10% of the stated value; ±9% of the stated value; ±8% of the stated value; ±7% of the stated value; ±6% of the stated value; ±5% of the stated value; ±4% of the stated value; ±3% of the stated value; ±2% of the stated value; or ±1% of the stated value.

Notwithstanding that the numerical ranges and parameters setting forth the broad scope of the invention are approximations, the numerical values set forth in the specific examples are reported as precisely as possible. Any numerical value, however, inherently contains certain errors necessarily resulting from the standard deviation found in their respective testing measurements.

The terms “a,” “an,” “the” and similar referents used in the context of describing the invention (especially in the context of the following claims) are to be construed to cover both the singular and the plural, unless otherwise indicated herein or clearly contradicted by context. Recitation of ranges of values herein is merely intended to serve as a shorthand method of referring individually to each separate value falling within the range. Unless otherwise indicated herein, each individual value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein is intended merely to better illuminate the invention and does not pose a limitation on the scope of the invention otherwise claimed. No language in the specification should be construed as indicating any non-claimed element essential to the practice of the invention.

Groupings of alternative elements or embodiments of the invention disclosed herein are not to be construed as limitations. Each group member may be referred to and claimed individually or in any combination with other members of the group or other elements found herein. It is anticipated that one or more members of a group may be included in, or deleted from, a group for reasons of convenience and/or patentability. When any such inclusion or deletion occurs, the specification is deemed to contain the group as modified thus fulfilling the written description of all Markush groups used in the appended claims.

Certain embodiments of this invention are described herein, including the best mode known to the inventors for carrying out the invention. Of course, variations on these described embodiments will become apparent to those of ordinary skill in the art upon reading the foregoing description. The inventor expects skilled artisans to employ such variations as appropriate, and the inventors intend for the invention to be practiced otherwise than specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

Furthermore, numerous references have been made to patents, printed publications, journal articles and other written text throughout this specification (referenced materials herein). Each of the referenced materials are individually incorporated herein by reference in their entirety for their referenced teaching.

In closing, it is to be understood that the embodiments of the invention disclosed herein are illustrative of the principles of the present invention. Other modifications that may be employed are within the scope of the invention. Thus, by way of example, but not of limitation, alternative configurations of the present invention may be utilized in accordance with the teachings herein. Accordingly, the present invention is not limited to that precisely as shown and described.

The particulars shown herein are by way of example and for purposes of illustrative discussion of the preferred embodiments of the present invention only and are presented in the cause of providing what is believed to be the most useful and readily understood description of the principles and conceptual aspects of various embodiments of the invention. In this regard, no attempt is made to show structural details of the invention in more detail than is necessary for the fundamental understanding of the invention, the description taken with the drawings and/or examples making apparent to those skilled in the art how the several forms of the invention may be embodied in practice.

Definitions and explanations used in the present disclosure are meant and intended to be controlling in any future construction unless clearly and unambiguously modified in the examples or when application of the meaning renders any construction meaningless or essentially meaningless. In cases where the construction of the term would render it meaningless or essentially meaningless, the definition should be taken from Webster's Dictionary, 3rd Edition or a dictionary known to those of ordinary skill in the art, such as the Oxford Dictionary of Biochemistry and Molecular Biology (Eds. Attwood T et al., Oxford University Press, Oxford, 2006).

What is claimed is:

1. An engineered expression construct (EEC) having a reprogramming factor (RF) coding sequence operably linked to a 5′ untranslated region (UTR) consisting of the sequence as set forth in SEQ ID NO: 3 and a 3′ UTR consisting of the sequence as set forth in SEQ ID NOs: 34, 37, 38, or 39.

2. An engineered expression construct (EEC) having a 5′ untranslated region (UTR) operably linked to a reprogramming factor (RF) coding sequence, wherein the 5′ UTR has the sequence as set forth in CAUACUCA in between a minimal promoter and a Kozak sequence.

3. The EEC of claim 2, wherein the minimal promoter is a T7 promoter.

4. The EEC of claim 3, wherein the T7 promoter has the sequence as set forth in GGGAGA.

5. The EEC of claim 2, wherein the Kozak sequence has the sequence as set forth in GCCRCC, wherein R is A or G.

6. The EEC of claim 2, wherein the 5′ UTR further comprises a start codon.

7. The EEC of claim 2, wherein the 5′ UTR has
(i) the sequence as set forth in SEQ ID NO: 1 operably linked to a start codon or
(ii) the sequence as set forth in SEQ ID NO: 2 operably linked to a start codon.

8. The EEC of claim 7, wherein the sequence as set forth in SEQ ID NO: 1 operably linked to a start codon has the sequence as set forth in SEQ ID NO: 3.

9. The EEC of claim 7, wherein the sequence as set forth in SEQ ID NO: 2 operably linked to a start codon has the sequence as set forth in SEQ ID NO: 4.

10. The EEC of claim 2, wherein the 5′ UTR is less than 30 nucleotides.

11. The EEC of claim 2, further comprising a 3′ UTR.

12. The EEC of claim 11, wherein the 3′ UTR comprises a stop codon, spacer, and a stem loop structure.

13. The EEC of claim 12, wherein the stop codon has the sequence UAA, UGA, or UAG.

14. The EEC of claim 12, wherein the spacer has the sequence [N₁₋₃]AUA or [N₁₋₃]AAA.

15. The EEC of claim 12, wherein the spacer has the sequence UGCAUA or UGCAAA.

16. The EEC of claim 12, wherein the stem loop structure has hybridizing sequences as set forth in a) CCUC and GAGG or b) CUCC and GGAG.

17. The EEC of claim 12, wherein the stem loop structure has a loop segment having at least 7 nucleotides.

18. The EEC of claim 12, wherein the stem loop structure has a loop segment having 7-15 nucleotides.

19. The EEC of claim 12, wherein the stem loop structure has a loop segment having the sequence as set forth in UAACGGUCUU (SEQ ID NO: 5).

20. The EEC of claim 11, wherein the 3′ UTR is less than 30 nucleotides.

21. The EEC of claim 11, wherein the 3′ UTR further comprises a polyadenine (polyA) tail.

22. The EEC of claim 21, wherein the polyA tail has 60 residues or less.

23. The EEC of claim 21, wherein the polyA tail has 40 residues.

24. The EEC of claim 11, wherein the 3′ UTR has the sequence as set forth in SEQ ID NO: 28, SEQ ID NO: 29, SEQ ID NO: 30, SEQ ID NO: 31, SEQ ID NO: 32, or SEQ ID NO: 33.

25. The EEC of claim 11, wherein the 3′ UTR has the sequence as set forth in SEQ ID NO: 34, SEQ ID NO: 35, or SEQ ID NO: 36.

26. The EEC of claim 11, wherein the 3′ UTR has the sequence as set forth in SEQ ID NO: 37, SEQ ID NO: 38, SEQ ID NO: 39, SEQ ID NO: 40, SEQ ID NO: 41, SEQ ID NO: 42, SEQ ID NO: 43, SEQ ID NO: 44, or SEQ ID NO: 45.

27. The EEC of claim 2, wherein the EEC comprises in vitro-synthesized messenger RNA (mRNA).

28. The EEC of claim 2, wherein the EEC does not comprise modified nucleosides.

29. The EEC of claim 2, wherein the EEC does not comprise microRNA binding sites.

30. The EEC of claim 2, wherein the EEC further comprises a coding sequence encoding an immune evading factor.

31. The EEC of claim 30, wherein the immune evading factor comprises B18R, E3, K3, NS1, or ORF8.

32. The EEC of claim 2, wherein the EEC does not encode immune evading factors.

33. The EEC of claim 2, formulated for administration to a subject.

34. The EEC of claim 2, wherein the reprograming factor comprises Oct4, Sox2, Klf4, Nanog, Myc, SV40 large T antigen (SV40Tag), or Lin28.

35. An enhancer sequence consisting of the sequence as set forth in CAUACUCA operably linked to a reprogramming factor coding sequence.

36. An engineered expression construct (EEC) having 1, 2, 3, 4, or 5 copies of the sequence as set forth in CAUACUCA operably linked to a reprogramming factor coding sequence.

37. The EEC of claim 36, wherein the enhancer sequence is operably linked to a promoter.

38. The EEC of claim 37 wherein the promoter is a minimal promoter.

39. The EEC of claim 38, wherein the minimal promoter is a T7 promoter.

40. The EEC of claim 39, wherein the T7 promoter comprises the sequence: GGAGA.

41. The EEC of claim 36, wherein the reprograming factor comprises Oct4, Sox2, Klf4, Nanog, Myc, SV40Tag, or Lin28.

42. An engineered expression construct (EEC) comprising an in vitro-synthesized RNA comprising a reprogramming factor (RF) coding sequence within an open reading frame that encodes one or more RF for translation in a mammalian cell, wherein said in vitro-synthesized RNA further comprises one of a 5′ untranslated region comprising CAUACUCA and a 3′ untranslated region comprising one of SEQ ID NO: 28, SEQ ID NO: 29, SEQ ID NO: 30, SEQ ID NO: 31, SEQ ID NO: 32, or SEQ ID NO: 33.

43. The EEC of claim 42, wherein the reprograming factor comprises Oct4, Sox2, Klf4, Nanog, Myc, SV40Tag, or Lin28.

44. An engineered expression construct (EEC) comprising an in vitro-synthesized RNA comprising a reprogramming factor (RF) coding sequence within an open reading frame that encodes one or more RF for translation in a mammalian cell, wherein said in vitro-synthesized RNA further comprises one of a 5′ untranslated region comprising SEQ ID NO: 1, or SEQ ID NO:
2 and a 3′ untranslated region comprising SEQ ID NO: 28, SEQ ID NO: 29, SEQ ID NO: 30, SEQ ID NO: 31, SEQ ID NO: 32, or SEQ ID NO: 33.

45. The EEC of claim 44, wherein the reprograming factor comprises Oct4, Sox2, Klf4, Nanog, Myc, SV40Tag, or Lin28.

46. An engineered expression construct (EEC) comprising an in vitro-synthesized RNA comprising an open reading frame that encodes one or more reprogramming factors (RF) for translation in a mammalian cell, wherein said in vitro-synthesized RNA further comprises a 5′ untranslated region comprising a T7 promoter, the sequence as set forth in CAUACUCA, and a Kozak sequence.

47. The EEC of claim 46, wherein the one or more RF comprise Oct4, Sox2, Klf4, Nanog, Myc, SV40Tag, or Lin28.

48. The EEC of claim 46, wherein the T7 promoter is selected from a T7 Class Ill promoter.

49. The EEC of claim 48, wherein the T7 Class III promoter comprises the sequence as set forth in GGGAGA.

50. The EEC of claim 46, wherein the Kozak sequence comprises the sequence as set forth in GCCRCC, wherein R is A or G.

51. The EEC of claim 46, further comprising a start codon.

52. An engineered expression construct (EEC) comprising an in vitro-synthesized RNA comprising an open reading frame that encodes one or more RF for translation in a mammalian cell, wherein said in vitro-synthesized RNA comprises a 3′ untranslated region comprising SEQ ID NO: 28, SEQ ID NO: 29, SEQ ID NO: 30, SEQ ID NO: 31, SEQ ID NO: 32, or SEQ ID NO: 33 and a stop codon.

53. The EEC of claim 52, wherein the one or more RF comprise Oct4, Sox2, Klf4, Nanog, Myc, SV40Tag, or Lin28.

54. The engineered expression construct of claim 52, wherein the stop codon is UAA, UAG, or UGA.

55. An engineered expression construct (EEC) comprising an in vitro-synthesized RNA comprising an open reading frame that encodes one or more RFs for translation in a mammalian cell, wherein said in vitro-synthesized RNA comprises a 3′ untranslated region comprising either a) CCUC and GAGG or b) CUCC and GGAG wherein either set of the 3′ untranslated region sequences is separated by no fewer than seven nucleotides.

56. The EEC of claim 55, wherein the one or more RF comprise Oct4, Sox2, Klf4, Nanog, Myc, SV40Tag, or Lin28.

57. The EEC of claim 55, further comprising a spacer.

58. The EEC of claim 57, wherein the spacer comprises the sequence N₁₋₃]AUA or [N₁₋₃]AAA.

59. The EEC of claim 57, wherein the spacer comprises the sequence UGCAUA or UGCAAA.

60. An engineered expression construct (EEC) having an open reading frame (ORF) and a 5′ untranslated region (UTR) and/or a 3′UTR, wherein
the ORF encodes a reprogramming factor (RF) and/or an immune evading factor (IEF);
the 5′ UTR comprises a minimal promoter, the sequence as set forth in CAUACUCA, and a Kozak sequence;
the 3′UTR comprises a stop codon, spacer, and a stem loop structure.

61. The EEC of claim 60, wherein the minimal promoter is a T7 promoter.

62. The EEC of claim 61, wherein the T7 promoter has the sequence as set forth in GGGAGA.

63. The EEC of claim 60, wherein the Kozak sequence has the sequence as set forth in GCCRCC, wherein R is A or G.

64. The EEC of claim 60, wherein the 5′ UTR further comprises a start codon.

65. The EEC of claim 60, wherein the 5′ UTR has
(iii) the sequence as set forth in SEQ ID NO: 1 operably linked to a start codon or
(iv) the sequence as set forth in SEQ ID NO: 2 operably linked to a start codon.

66. The EEC of claim 65, wherein the sequence as set forth in SEQ ID NO: 1 operably linked to a start codon has the sequence as set forth in SEQ ID NO: 3.

67. The EEC of claim 65, wherein the sequence as set forth in SEQ ID NO: 2 operably linked to a start codon has the sequence as set forth in SEQ ID NO: 4.

68. The EEC of claim 60, wherein the 3′ UTR further comprises a stop codon.

69. The EEC of claim 68, wherein the stop codon has the sequence UAA, UGA, or UAG.

70. The EEC of claim 60, wherein the spacer has the sequence [N1.3] AUA or [N1-3] AAA.

71. The EEC of claim 60, wherein the spacer has the sequence UGCAUA or UGCAAA.

72. The EEC of claim 60, wherein the stem loop structure has hybridizing sequences as set forth in a) CCUC and GAGG or b) CUCC and GGAG.

73. The EEC of claim 60, wherein the stem loop structure has a loop segment having at least 7 nucleotides.

74. The EEC of claim 60, wherein the stem loop structure has a loop segment having 7-15 nucleotides.

75. The EEC of claim 60, wherein the stem loop structure has a loop segment having the sequence as set forth in UAACGGUCUU (SEQ ID NO: 5).

76. The EEC of claim 60, wherein the 3′ UTR further comprises a polyadenine (polyA) tail.

77. The EEC of claim 60, wherein the 3′ UTR has the sequence as set forth in SEQ ID NO: 28, SEQ ID NO: 29, SEQ ID NO: 30, SEQ ID NO: 31, SEQ ID NO: 32, or SEQ ID NO: 33.

78. The EEC of claim 60, wherein the 3′ UTR has the sequence as set forth in SEQ ID NO: 34, SEQ ID NO: 35, or SEQ ID NO: 36.

79. The EEC of claim 60, wherein the 3′ UTR has the sequence as set forth in SEQ ID NO: 37, SEQ ID NO: 38, SEQ ID NO: 39, SEQ ID NO: 40, SEQ ID NO: 41, SEQ ID NO: 42, SEQ ID NO: 43, SEQ ID NO: 44, or SEQ ID NO: 45.

80. The EEC of claim 60, wherein the reprograming factor comprises Oct4, Sox2, Klf4, Nanog, cMyc, SV40 large T antigen (SV40Tag), and/or Lin28.

81. The EEC of claim 80, wherein the EEC comprises the sequence as set forth in SEQ ID NO: 84, SEQ ID NO: 86, SEQ ID NO: 89, SEQ ID NO: 85, SEQ ID NO: 87, SEQ ID NO: 90, SEQ ID NO: 88, SEQ ID NO: 105, SEQ ID NO: 106, or SEQ ID NO: 107.

82. The EEC of claim 60, wherein the IEF comprises B18R, E3, and/or K3.

83. The EEC of claim 82, wherein the EEC comprises the sequence as set forth in SEQ ID NO: 91.

84. The EEC of claim 82, wherein the EEC comprises the sequence as set forth in SEQ ID NO: 92.

85. The EEC of claim 82, wherein the EEC comprises the sequence as set forth in SEQ ID NO: 93.

86. A method for reprogramming somatic cells into induced pluripotent stem cells (iPSC) comprising: contacting somatic cells with the EEC of any of claims 1, 2, 36, 42, 44, 46, 52, or 55.

87. A method for reprogramming somatic cells into induced pluripotent stem cells (iPSC) comprising: contacting somatic cells with an engineered expression construct (EEC) encoding a reprogramming factor (RF) operably linked to
i) a 5′ untranslated region (UTR) comprising a minimal promoter, a mini-enhancer, and a Kozak sequence; or
ii) a 3′ UTR comprising a spacer and a stem loop structure.

88. The method of claim 87, wherein the RF comprises Oct4, Sox2, Klf4, Nanog, Myc, SV40Tag, or Lin28.

89. The method of claim 87, wherein the minimal promoter comprises a T7 promoter.

90. The method of claim 89, wherein the T7 promoter comprises the sequence as set forth in GGGAGA.

91. The method of claim 87, wherein the mini-enhancer comprises the sequence as set forth in CAUACUCA.

92. The method of claim 87, wherein the Kozak sequence comprises the sequence as set forth in GCCRCC wherein R is A or G.

93. The method of claim 92, wherein the Kozak sequence comprises the sequence as set forth in GCCACC or GCCGCC.

94. The method of claim 87, wherein the 5′ UTR comprises the sequence as set forth in SEQ ID NO: 1 or SEQ ID NO: 2.

95. The method of claim 87, wherein the 5′ UTR further comprises a start codon.

96. The method of claim 87, wherein the 5′ UTR comprises the sequence as set forth in SEQ ID NO: 3 or SEQ ID NO: 4.

97. The method of claim 87, wherein the spacer comprises a sequence as set forth in [N₁₋₃]AUA or [N₁₋₃]AAA.

98. The method of claim 87, wherein the spacer comprises a sequence as set forth in UGCAUA or UGCAAA.

99. The method of claim 87, wherein the stem loop structure comprises two hybridizing sequences and a loop segment.

100. The method of claim 99, wherein the two hybridizing sequences comprise i) CCUC and GAGG, or ii) CUCC and GGAG.

101. The method of claim 99, wherein the loop segment comprises at least 7 nucleotides.

102. The method of claim 99, wherein the loop segment comprises 7-15 nucleotides.

103. The method of claim 99, wherein the loop segment comprises the sequence as set forth in SEQ ID NO: 5.

104. The method of claim 87, wherein the stem loop structure comprises the sequence as set forth in SEQ ID NO: 8, SEQ ID NO: 9, SEQ ID NO: 10, SEQ ID NO: 11, SEQ ID NO: 12, SEQ ID NO: 13, SEQ ID NO: 14, SEQ ID NO: 15, SEQ ID NO: 16, SEQ ID NO: 17, SEQ ID NO: 18, SEQ ID NO: 19, SEQ ID NO: 20, SEQ ID NO: 21, SEQ ID NO: 22, SEQ ID NO: 23, SEQ ID NO: 24, SEQ ID NO: 25, SEQ ID NO: 26, or SEQ ID NO: 27.

105. The method of claim 87, wherein the 3′ UTR comprises the sequence as set forth in SEQ ID NO: 28, SEQ ID NO: 29, SEQ ID NO: 30, SEQ ID NO: 31, SEQ ID NO: 32, SEQ ID NO: 33, SEQ ID NO: 34, SEQ ID NO: 35, or SEQ ID NO: 36.

106. The method of claim 87, wherein the 3′ UTR further comprises a stop codon.

107. The method of claim 106, wherein the stop codon comprises the sequence as set forth in UAA, UAG, or UGA.

108. The method of claim 87, wherein the 3′ UTR comprises the sequence as set forth in SEQ ID NO: 37, SEQ ID NO: 38, SEQ ID NO: 39, SEQ ID NO: 40, SEQ ID NO: 41, SEQ ID NO: 42, SEQ ID NO: 43, SEQ ID NO: 44, or SEQ ID NO: 45.

109. The method of claim 87, wherein the somatic cells comprise mammalian cells.

110. The method of claim 87, wherein the somatic cells comprise somatic cells.

111. The method of claim 87, wherein the somatic cells comprise fibroblasts, hepatocytes, myoblasts, neurons, osteoblasts, osteoclasts, kidney cells, immune cells, or non-pluripotent stem cells.

112. The method of claim 111, wherein the fibroblasts comprise adult dermal fibroblasts or human foreskin fibroblasts.

113. The method of claim 111, wherein the kidney cells comprise HEK293 cells.

114. The method of claim 111, wherein the immune cells comprise T cells.

115. The method of claim 111, wherein the non-pluripotent stem cells comprise hematopoietic stem cells or mesenchymal stem cells.

116. The method of claim 111, wherein the hematopoietic stem cells comprise CD34+ cells.

117. The method of claim 87, further comprising electroporating the somatic cells with mRNA encoding an immune evasion factor (IEF).

118. The method of claim 117, wherein the IEF comprises B18R, E3, K3, NS1, or ORF8.

119. The method of claim 117, wherein the mRNA encoding the IEF comprises a 5′UTR comprising the sequence as set forth in SEQ ID NO: 1 and a 3′UTR comprising the sequence as set forth in SEQ ID NO: 34.

120. The method of claim 119, wherein the mRNA encoding the IEF comprises the sequence as set forth in SEQ ID NO: 91, SEQ ID NO: 92, or SEQ ID NO: 93.

121. The method of claim 87, further comprising contacting the somatic cells with miRNA.

122. The method of claim 121, wherein the miRNA comprise miR-302a, miR-302b, miR-302c, miR-302d, and miR-367.

123. The method of claim 121, wherein the miR-302a comprises the sequence as set forth in SEQ ID NO: 64.

124. The method of claim 121, wherein the miR-302b comprises the sequence as set forth in SEQ ID NO: 65.

125. The method of claim 121, wherein the miR-302a comprises the sequence as set forth in SEQ ID NO: 66.

126. The method of claim 121, wherein the miR-302a comprises the sequence as set forth in SEQ ID NO: 67.

127. The method of claim 121, wherein the miR-367 comprises the sequence as set forth in SEQ ID NO: 68.

128. The method of claim 87, further comprising contacting the somatic cells with an IEF protein.

129. The method of claim 128, wherein the IEF protein comprises B18R, E3, K3, NS1, or ORF8.

130. The method of claim 87, wherein the method comprises contacting the isolated somatic cells with the EEC daily for 10 days or less, 9 days or less, 8 days or less, 7 days or less, 6 days or less, 5 days or less, 4 days or less.

131. The method of claim 87, wherein the method comprises contacting the isolated somatic cells with the EEC daily for 4 days.

132. The method of claim 87, wherein the method does not use feeder cells.

133. An induced pluripotent stem cell (iPSC) made according to the method of claim 86 or 87.

134. An engineered expression construct (EEC) comprising the sequence as set forth in SEQ ID NO: 71, SEQ ID NO: 72, SEQ ID NO: 73, SEQ ID NO: 74, SEQ ID NO: 75, SEQ ID NO: 76, SEQ ID NO: 77, SEQ ID NO: 78, SEQ ID NO: 79, SEQ ID NO: 80, SEQ ID NO: 81, SEQ ID NO: 84, SEQ ID NO: 85, SEQ ID NO: 86, SEQ ID NO: 87, SEQ ID NO: 88, SEQ ID NO: 89, SEQ ID NO: 90, SEQ ID NO: 91, SEQ ID NO: 92, SEQ ID NO: 93, SEQ ID NO: 105, SEQ ID NO: 106, or SEQ ID NO: 107.

135. A cDNA encoding the EEC of claim 1, 2, 36, 42, 44, 46, 52, 55, or 134.


SEQUENCE LISTING (107 sequences)

SEQ ID NO: 1 - RNA, 20 nt, synthetic construct
  source 1..20: mol_type = other RNA
gggagacatactcagccacc

SEQ ID NO: 2 - RNA, 20 nt, synthetic construct
  source 1..20: mol_type = other RNA
gggagacatactcagccgcc

SEQ ID NO: 3 - RNA, 23 nt, synthetic construct
  source 1..23: mol_type = other RNA
gggagacatactcagccaccatg

SEQ ID NO: 4 - RNA, 23 nt, synthetic construct
  source 1..23: mol_type = other RNA
gggagacatactcagccgccatg

SEQ ID NO: 5 - RNA, 10 nt, synthetic construct
  source 1..10: mol_type = other RNA
taacggtctt

SEQ ID NO: 6 - RNA, 10 nt, synthetic construct
  source 1..10: mol_type = other RNA
gccrccrtgg

SEQ ID NO: 7 - DNA, 18 bp, synthetic construct
  source 1..18: mol_type = other DNA
taatacgactcactatag

SEQ ID NO: 8 - RNA, 28 nt, synthetic construct
  source 1..28: mol_type = other RNA
gatgccccattcacgagtagtgggtatt

SEQ ID NO: 9 - RNA, 28 nt, synthetic construct
  source 1..28: mol_type = other RNA
ggcaccctgcgcaggtgatgcaggtgcc

SEQ ID NO: 10 - RNA, 28 nt, synthetic construct
  source 1..28: mol_type = other RNA
gttcgctcggtcaggagagctgacggac

SEQ ID NO: 11 - RNA, 28 nt, synthetic construct
  source 1..28: mol_type = other RNA
tcttacagtggcatgtgaccgtttaagg

SEQ ID NO: 12 - RNA, 28 nt, synthetic construct
  source 1..28: mol_type = other RNA
cgcggcgcatgcacgtgacatgcctgcg

SEQ ID NO: 13 - RNA, 28 nt, synthetic construct
  source 1..28: mol_type = other RNA
cggtcccgtggcaagagtctatggattg

SEQ ID NO: 14 - RNA, 28 nt, synthetic construct
  source 1..28: mol_type = other RNA
atgttcggctccaagagcgagttgatat

SEQ ID NO: 15 - RNA, 28 nt, synthetic construct
  source 1..28: mol_type = other RNA
cgattcgggcacatgtgctgtctgattg

SEQ ID NO: 16 - RNA, 28 nt, synthetic construct
  source 1..28: mol_type = other RNA
gtattctgatgcacgtgccatcaagtac

SEQ ID NO: 17 - RNA, 28 nt, synthetic construct
  source 1..28: mol_type = other RNA
ttgagcaggatcaagtgcattctttcaa

SEQ ID NO: 18 - RNA, 28 nt, synthetic construct
  source 1..28: mol_type = other RNA
rryryyyyryyyryrrryrryrrryryy

SEQ ID NO: 19 - RNA, 28 nt, synthetic construct
  source 1..28: mol_type = other RNA
rryryyyyryryrrryrryryrrryryy

SEQ ID NO: 20 - RNA, 28 nt, synthetic construct
  source 1..28: mol_type = other RNA
ryyyryyyrryyrrrrrrryyrryrrry

SEQ ID NO: 21 - RNA, 28 nt, synthetic construct
  source 1..28: mol_type = other RNA
yyyyryrryrryryryrryyryyyrrrr

SEQ ID NO: 22 - RNA, 28 nt, synthetic construct
  source 1..28: mol_type = other RNA
yryrryryryryryryrryryryyyryr

SEQ ID NO: 23 - RNA, 28 nt, synthetic construct
  source 1..28: mol_type = other RNA
yrryyyyryrryrrrrryyyryrrryyr

SEQ ID NO: 24 - RNA, 28 nt, synthetic construct
  source 1..28: mol_type = other RNA
ryryyyrryyyyrrrrryrrryyrryry

SEQ ID NO: 25 - RNA, 28 nt, synthetic construct
  source 1..28: mol_type = other RNA
yrryyyrrryryryryryyryyyrryyr

SEQ ID NO: 26 - RNA, 28 nt, synthetic construct
  source 1..28: mol_type = other RNA
ryryyyyrryryryryryyryyrrryry

SEQ ID NO: 27 - RNA, 28 nt, synthetic construct
  source 1..28: mol_type = other RNA
yyrrryrrrryyrrryryryyyyyyyrr

SEQ ID NO: 28 - RNA, 29 nt, synthetic construct
  source 1..29: mol_type = other RNA
  misc_feature 2..3: note = nucleotide n can be either present or absent
  misc_difference 18..25: note = nucleotide n can be either present or absent
nnnatacctcnnnnnnnnnnnnnnngagg

SEQ ID NO: 29 - RNA, 29 nt, synthetic construct
  source 1..29: mol_type = other RNA
  misc_feature 2..3: note = nucleotide "n" can be present or absent
  misc_difference 18..25: note = nucleotide "n" can be present or absent
nnnaaacctcnnnnnnnnnnnnnnngagg

SEQ ID NO: 30 - RNA, 29 nt, synthetic construct
  source 1..29: mol_type = other RNA
  misc_difference 2..3: note = nucleotide "n" can be present or absent
  misc_difference 18..25: note = nucleotide "n" can be present or absent
nnnataggagnnnnnnnnnnnnnnnctcc

SEQ ID NO: 31 - RNA, 29 nt, synthetic construct
  source 1..29: mol_type = other RNA
  misc_difference 2..3: note = nucleotide "n" can be present or absent
  misc_difference 18..25: note = nucleotide "n" can be present or absent
nnnaaaggagnnnnnnnnnnnnnnnctcc

SEQ ID NO: 32 - RNA, 29 nt, synthetic construct
  source 1..29: mol_type = other RNA
  misc_difference 18..25: note = nucleotide "n" can be present or absent
  misc_difference 2..3: note = nucleotide "n" can be present or absent
nnnatagaggnnnnnnnnnnnnnnncctc

SEQ ID NO: 33 - RNA, 29 nt, synthetic construct
  source 1..29: mol_type = other RNA
  misc_difference 2..3: note = nucleotide "n" can be present or absent
  misc_difference 18..25: note = nucleotide "n" can be present or absent
nnnaaagaggnnnnnnnnnnnnnnncctc

SEQ ID NO: 34 - RNA, 24 nt, synthetic construct
  source 1..24: mol_type = other RNA
tgcatacctctaacggtcttgagg

SEQ ID NO: 35 - RNA, 24 nt, synthetic construct
  source 1..24: mol_type = other RNA
tgcataggagtaacggtcttctcc

SEQ ID NO: 36 - RNA, 24 nt, synthetic construct
  source 1..24: mol_type = other RNA
tgcaaaggagtaacggtcttctcc

SEQ ID NO: 37 - RNA, 27 nt, synthetic construct
  source 1..27: mol_type = other RNA
taatgcatacctctaacggtcttgagg

SEQ ID NO: 38 - RNA, 27 nt, synthetic construct
  source 1..27: mol_type = other RNA
tgatgcatacctctaacggtcttgagg

SEQ ID NO: 39 - RNA, 27 nt, synthetic construct
  source 1..27: mol_type = other RNA
tagtgcatacctctaacggtcttgagg

SEQ ID NO: 40 - RNA, 27 nt, synthetic construct
  source 1..27: mol_type = other RNA
taatgcatagaggtaacggtcttcctc

SEQ ID NO: 41 - RNA, 27 nt, synthetic construct
  source 1..27: mol_type = other RNA
tgatgcatagaggtaacggtcttcctc

SEQ ID NO: 42 - RNA, 27 nt, synthetic construct
  source 1..27: mol_type = other RNA
tagtgcatagaggtaacggtcttcctc

SEQ ID NO: 43 - RNA, 27 nt, synthetic construct
  source 1..27: mol_type = other RNA
taatgcaaagaggtaacggtcttcctc

SEQ ID NO: 44 - RNA, 27 nt, synthetic construct
  source 1..27: mol_type = other RNA
tgatgcaaagaggtaacggtcttcctc

SEQ ID NO: 45 - RNA, 27 nt, synthetic construct
  source 1..27: mol_type = other RNA
tagtgcaaagaggtaacggtcttcctc

SEQ ID NO: 46 - DNA, 23 bp, synthetic construct
  source 1..23: mol_type = other DNA
taatacgactcactatagggaga

SEQ ID NO: 47 - DNA, 10 bp, synthetic construct
  source 1..10: mol_type = other DNA
gccrccatgg

SEQ ID NO: 48 - DNA, 32 bp, synthetic construct
  source 1..32: mol_type = other DNA
gactcctaatacgactcactatagggagaatg

SEQ ID NO: 49 - DNA, 38 bp, synthetic construct
  source 1..38: mol_type = other DNA
gactcctaatacgactcactatagggagagccaccatg

SEQ ID NO: 50 - DNA, 46 bp, synthetic construct
  source 1..46: mol_type = other DNA
gactcctaatacgactcactatagggagacatactcagccaccatg

SEQ ID NO: 51 - DNA, 64 bp, synthetic construct
  source 1..64: mol_type = other DNA
ttttttttttttttttttttttttttttttttttttttttcctcaagaccgttagaggtatgca

SEQ ID NO: 52 - DNA, 64 bp, synthetic construct
  source 1..64: mol_type = other DNA
ttttttttttttttttttttttttttttttttttttttttggagaagaccgttactcctatgca

SEQ ID NO: 53 - DNA, 64 bp, synthetic construct
  source 1..64: mol_type = other DNA
ttttttttttttttttttttttttttttttttttttttttggagaagaccgttactcctttgca

SEQ ID NO: 54 - RNA, 18 nt, synthetic construct
  source 1..18: mol_type = other RNA
cctctaacggtcttgagg

SEQ ID NO: 55 - RNA, 18 nt, synthetic construct
  source 1..18: mol_type = other RNA
gaggtaacggtcttcctc

SEQ ID NO: 56 - RNA, 18 nt, synthetic construct
  source 1..18: mol_type = other RNA
ctcctaacggtcttggag

SEQ ID NO: 57 - RNA, 18 nt, synthetic construct
  source 1..18: mol_type = other RNA
ggagtaacggtcttctcc

SEQ ID NO: 58 - RNA, 15 nt, synthetic construct
  source 1..15: mol_type = other RNA
cctctaactgtgagg

SEQ ID NO: 59 - RNA, 17 nt, synthetic construct
  source 1..17: mol_type = other RNA
gaggtaacgctctcctc

SEQ ID NO: 60 - RNA, 19 nt, synthetic construct
  source 1..19: mol_type = other RNA
ctcctaacggtcgtgggag

SEQ ID NO: 61 - RNA, 20 nt, synthetic construct
  source 1..20: mol_type = other RNA
ggagggtaaccgtcttctcc

SEQ ID NO: 62 - RNA, 21 nt, synthetic construct
  source 1..21: mol_type = other RNA
cctctaacggtcttagcgagg

SEQ ID NO: 63 - RNA, 23 nt, synthetic construct
  source 1..23: mol_type = other RNA
gaggtaacgtaacggtcttcctc

SEQ ID NO: 64 - RNA, 23 nt, synthetic construct
  source 1..23: mol_type = other RNA
taagtgcttccatgttttggtga

SEQ ID NO: 65 - RNA, 23 nt, synthetic construct
  source 1..23: mol_type = other RNA
taagtgcttccatgttttagtag

SEQ ID NO: 66 - RNA, 23 nt, synthetic construct
  source 1..23: mol_type = other RNA
taagtgcttccatgtttcagtgg

SEQ ID NO: 67 - RNA, 23 nt, synthetic construct
  source 1..23: mol_type = other RNA
taagtgcttccatgtttgagtgt

SEQ ID NO: 68 - RNA, 22 nt, synthetic construct
  source 1..22: mol_type = other RNA
aattgcactttagcaatggtga

SEQ ID NO: 69 - DNA, 827 bp, synthetic construct
  source 1..827: mol_type = other DNA
gactcctaatacgactcactatagggagacatactcagccaccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaatgcatacctctaacggtcttgaggaaaaaaaaaaaaaaaaaaaaaaaaaaaaaaaaaaaaaaaa

SEQ ID NO: 70 - DNA, 707 bp, synthetic construct
  source 1..707: mol_type = other DNA
gactcctaatacgactcactatagggagacatactcagccaccatgaccgaatataagccgactgttagactcgcgactcgcgacgacgttccgagagctgtaaggacgctcgcggctgcgttcgcggactatcccgccactaggcacactgtagacccggaccgacacatagagagagtgacggaactccaagagttgtttttgacgagggttgggctcgatattgggaaggtctgggttgcggacgacggtgcagcagtggctgtgtggaccacaccggagagtgtcgaagcgggagctgttttcgcagaaataggaccgaggatggctgaattgtcaggatcccggttggcagcgcaacaacagatggaaggcctccttgccccacacagaccaaaagagcccgcatggttccttgccacggtaggtgtttccccagatcaccaaggaaaaggccttgggtcagctgttgtattgcctggggtagaagccgccgagcgcgcaggagttccagcttttcttgaaacgtctgcgcctcgcaatctccccttctacgagcgactgggatttacggtgactgcagatgtagaggttcctgagggtccccgaacatggtgcatgacgcgaaaaccgggtgcatagtgcatacctctaacggtcttgaggaaaaaaaaaaaaaaaaaaaaaaaaaaaaaaaaaaaaaaaa

SEQ ID NO: 71 - DNA, 1190 bp, synthetic construct
  source 1..1190: mol_type = other DNA
gactcctaatacgactcactatagggagacatactcagccaccatggcgggacacctggcttcggatttcgccttctcgccccctccaggtggtggaggtgatgggccaggggggccggagccgggctgggttgatcctcggacctggctaagcttccaaggccctcctggagggccaggaatcgggccgggggttgggccaggctctgaggtgtgggggattcccccatgccccccgccgtatgagttctgtggggggatggcgtactgtgggccccaggttggagtggggctagtgccccaaggcggcttggagacctctcagcctgagggcgaagcaggagtcggggtggagagcaactccgatggggcctccccggagccctgcaccgtcacccctggtgccgtgaagctggagaaggagaagctggagcaaaacccggaggagtcccaggacatcaaagctctgcagaaagaactcgagcaatttgccaagctcctgaagcagaagaggatcaccctgggatatacacaggccgatgtggggctcaccctgggggttctatttgggaaggtattcagccaaacgaccatctgccgctttgaggctctgcagcttagcttcaagaacatgtgtaagctgcggcccttgctgcagaagtgggtggaggaagctgacaacaatgaaaatcttcaggagatatgcaaagcagaaaccctcgtgcaggcccgaaagagaaagcgaaccagtatcgagaaccgagtgagaggcaacctggagaatttgttcctgcagtgcccgaaacccacactgcagcagatcagccacatcgcccagcagcttgggctcgagaaggatgtggtccgagtgtggttctgtaaccggcgccagaagggcaagcgatcaagcagcgactatgcacaacgagaggattttgaggctgctgggtctcctttctcagggggaccagtgtcctttcctctggccccagggccccattttggtaccccaggctatgggagccctcacttcactgcactgtactcctcggtccctttccctgagggggaagcctttccccctgtctccgtcaccactctgggctctcccatgcattcaaactgatgcatacctctaacggtcttgaggaaaaaaaaaaaaaaaaaaaaaaaaaaaaaaaaaaaaaaaa

SEQ ID NO: 72 - DNA, 1025 bp, synthetic construct
  source 1..1025: mol_type = other DNA
gactcctaatacgactcactatagggagacatactcagccaccatgagtgtggatccagcttgtccccaaagcttgccttgctttgaagcatccgactgtaaagaatcttcacctatgcctgtgatttgtgggcctgaagaaaactatccatccttgcaaatgtcttctgctgagatgcctcacacggagactgtctctcctcttccttcctccatggatctgcttattcaggacagccctgattcttccaccagtcccaaaggcaaacaacccacttctgcagagaagagtgtcgcaaaaaaggaagacaaggtcccggtcaagaaacagaagaccagaactgtgttctcttccacccagctgtgtgtactcaatgatagatttcagagacagaaatacctcagcctccagcagatgcaagaactctccaacatcctgaacctcagctacaaacaggtgaagacctggttccagaaccagagaatgaaatctaagaggtggcagaaaaacaactggccgaagaatagcaatggtgtgacgcagaaggcctcagcacctacctaccccagcctttactcttcctaccaccagggatgcctggtgaacccgactgggaaccttccaatgtggagcaaccagacctggaacaattcaacctggagcaaccagacccagaacatccagtcctggagcaaccactcctggaacactcagacctggtgcacccaatcctggaacaatcaggcctggaacagtcccttctataactgtggagaggaatctctgcagtcctgcatgcagttccagccaaattctcctgccagtgacttggaggctgccttggaagctgctggggaaggccttaatgtaatacagcagaccactaggtattttagtactccacaaaccatggatttattcctaaactactccatgaacatgcaacctgaagacgtgtgatgcatacctctaacggtcttgaggaaaaaaaaaaaaaaaaaaaaaaaaaaaaaaaaaaaaaaaa

SEQ ID NO: 73 - DNA, 1427 bp, synthetic construct
  source 1..1427: mol_type = other DNA
gactcctaatacgactcactatagggagacatactcagccaccatgcccctcaacgttagcttcaccaacaggaactatgacctcgactacgactcggtgcagccgtatttctactgcgacgaggaggagaacttctaccagcagcagcagcagagcgagctgcagcccccggcgcccagcgaggatatctggaagaaattcgagctgctgcccgccccgcccctgtcccctagccgccgctccgggctctgctcgccctcctacgttgcggtcacacccttctcccttcggggagacaacgacggcggtggcgggagcttctccacggccgaccagctggagatggtgaccgagctgctgggaggagacatggtgaaccagagtttcatctgcgacccggacgacgagaccttcatcaaaaacatcatcatccaggactgtatgtggagcggcttctcggccgccgccaagctcgtctcagagaagctggcctcctaccaggctgcgcgcaaagacagcggcagcccgaaccccgcccgcggccacagcgtctgctccacctccagcttgtacctgcaggatctgagcgccgccgcctcagagtgcatcgacccctcggtggtcttcccctaccctctcaacgacagcagctcgcccaagtcctgcgcctcgcaagactccagcgccttctctccgtcctcggattctctgctctcctcgacggagtcctccccgcagggcagccccgagcccctggtgctccatgaggagacaccgcccaccaccagcagcgactctgaggaggaacaagaagatgaggaagaaatcgatgttgtttctgtggaaaagaggcaggctcctggcaaaaggtcagagtctggatcaccttctgctggaggccacagcaaacctcctcacagcccactggtcctcaagaggtgccacgtctccacacatcagcacaactacgcagcgcctccctccactcggaaggactatcctgctgccaagagggtcaagttggacagtgtcagagtcctgagacagatcagcaacaaccgaaaatgcaccagccccaggtcctcggacaccgaggagaatgtcaagaggcgaacacacaacgtcttggagcgccagaggaggaacgagctaaaacggagcttttttgccctgcgtgaccagatcccggagttggaaaacaatgaaaaggcccccaaggtagttatccttaaaaaagccacagcatacatcctgtccgtccaagcagaggagcaaaagctcatttctgaagaggacttgttgcggaaacgacgagaacagttgaaacacaaacttgaacagctacggaactcttgtgcgtaatgcatacctctaacggtcttgaggaaaaaaaaaaaaaaaaaaaaaaaaaaaaaaaaaaaaaaaa

SEQ ID NO: 74 - DNA, 2234 bp, synthetic construct
  source 1..2234: mol_type = other DNA
gactcctaatacgactcactatagggagacatactcagccaccatggataaagttttaaacagagaggaatctttgcagctaatggaccttctaggtcttgaaaggagtgcctgggggaatattcctctgatgagaaaggcatatttaaaaaaatgcaaggagtttcatcctgataaaggaggagatgaagaaaaaatgaagaaaatgaatactctgtacaagaaaatggaagatggagtaaaatatgctcatcaacctgactttggaggcttctgggatgcaactgagattccaacctatggaactgatgaatgggagcagtggtggaatgcctttaatgaggaaaacctgttttgctcagaagaaatgccatctagtgatgatgaggctactgctgactctcaacattctactcctccaaaaaagaagagaaaggtagaagaccccaaggactttccttcagaattgctaagttttttgagtcatgctgtgtttagtaatagaactcttgcttgctttgctatttacaccacaaaggaaaaagctgcactgctatacaagaaaattatggaaaaatattctgtaacctttataagtaggcataacagttataatcataacatactgttttttcttactccacacaggcatagagtgtctgctattaataactatgctcaaaaattgtgtacctttagctttttaatttgtaaaggggttaataaggaatatttgatgtatagtgccttgactagagatccattttctgttattgaggaaagtttgccaggtgggttaaaggagcatgattttaatccagaagaagcagaggaaactaaacaagtgtcctggaagcttgtaacagagtatgcaatggaaacaaaatgtgatgatgtgttgttattgcttgggatgtacttggaatttcagtacagttttgaaatgtgtttaaaatgtattaaaaaagaacagcccagccactataagtaccatgaaaagcattatgcaaatgctgctatatttgctgacagcaaaaaccaaaaaaccatatgccaacaggctgttgatactgttttagctaaaaagcgggttgatagcctacaattaactagagaacaaatgttaacaaacagatttaatgatcttttggataggatggatataatgtttggttctacaggctctgctgacatagaagaatggatggctggagttgcttggctacactgtttgttgcccaaaatggattcagtggtgtatgactttttaaaatgcatggtgtacaacattcctaaaaaaagatactggctgtttaaaggaccaattgatagtggtaaaactacattagcagctgctttgcttgaattatgtggggggaaagctttaaatgttaatttgcccttggacaggctgaactttgagctaggagtagctattgaccagtttttagtagtttttgaggatgtaaagggcactggaggggagtccagagatttgccttcaggtcagggaattaataacctggacaatttaagggattatttggatggcagtgttaaggtaaacttagaaaagaaacacctaaataaaagaactcaaatatttccccctggaatagtcaccatgaatgagtacagtgtgcctaaaacactgcaggccagatttgtaaaacaaatagattttaggcccaaagattatttaaagcattgcctggaacgcagtgagtttttgttagaaaagagaataattcaaagtggcattgctttgcttcttatgttaatttggtacagacctgtggctgagtttgctcaaagtattcagagcagaattgtggagtggaaagagagattggacaaagagtttagtttgtcagtgtatcaaaaaatgaagtttaatgtggctatgggaattggagttttagattggctaagaaacagtgatgatgatgatgaagacagccaggaaaatgctgataaaaatgaagatggtggggagaagaacatggaagactcagggcatgaaacaggcattgattcacagtcccaaggctcatttcaggcccctcagtcctcacagtctgttcatgatcataatcagccataccacatttgtagaggttttacttgctttaaaaaacctcccacacctccccctgaacctgaaacataatgcatacctctaacggtcttgaggaaaaaaaaaaaaaaaaaaaaaaaaaaaaaaaaaaaaaaaa

SEQ ID NO: 75 - DNA, 1166 bp, synthetic construct
  source 1..1166: mol_type = other DNA
gactcctaatacgactcactatagggagacatactcagccaccatgggcacgatgaaaatgatggtacatatatatttcgtatcattattgttattgctattccacagttacgccatagacatcgaaaatgaaatcacagaattcttcaataaaatgagagatactctaccagctaaagactctaaatggttgaatccagcatgtatgttcggaggcacaatgaatgatatagccgctctaggagagccattcagcgcaaagtgtcctcctattgaagacagtcttttatcgcacagatataaagactatgtggttaaatgggaaaggctagaaaaaaatagacggcgacaggtttctaataaacgtgttaaacatggtgatttatggatagccaactatacatctaaattcagtaaccgtaggtatttgtgcaccgtaactacaaagaatggtgactgtgttcagggtatagttagatctcatattagaaaacctccttcatgcattccaaaaacatatgaactaggtactcatgataagtatggcatagacttatactgtggaattctttacgcaaaacattataataatataacttggtataaagataataaggaaattaatatcgacgacattaagtattcacaaacgggaaaggaattaattattcataatccagagttagaagatagcggaagatacgactgttacgttcattacgacgacgttagaatcaagaatgatatcgtagtatcaagatgtaaaatacttacggttataccgtcacaagaccacaggtttaaactaatactagatccaaaaatcaacgtaacgataggagaacctgccaatataacatgcactgctgtgtcaacgtcattattgattgacgatgtactgattgaatgggaaaatccatccggatggcttataggattcgattttgatgtatactctgttttaactagtagaggcggtattaccgaggcgaccttgtactttgaaaatgttactgaagaatatataggtaatacatataaatgtcgtggacacaactattattttgaaaaaacccttacaactacagtagtattggagtaatgcatacctctaacggtcttgaggaaaaaaaaaaaaaaaaaaaaaaaaaaaaaaaaaaaaaaaa

SEQ ID NO: 76 - DNA, 680 bp, synthetic construct
  source 1..680: mol_type = other DNA
gactcctaatacgactcactatagggagacatactcagccaccatgtctaaaatctatatcgacgagcgttctaacgcagagattgtgtgtgaggctattaaaaccattggaatcgaaggagctactgctgcacaactaactagacaacttaatatggagaagcgagaagttaataaagctctgtacgatcttcaacgtagtgctatggtgtacagctccgacgatattcctcctcgttggtttatgacaacggaggcggataagccggatgctgatgctatggctgacgtcataatagatgatgtatcccgcgaaaaatcaatgagagaggatcataagtcttttgatgatgttattccggctaaaaaaattattgattggaaaggtgctaaccctgtcaccgttattaatgagtactgccaaattactaggagagattggtcttttcgtattgaatcagtggggcctagtaactctcctacattttatgcctgtgtagacatcgacggaagagtattcgataaggcagatggaaaatctaaacgagatgctaaaaataatgcagctaaattggcagtagataaacttcttggttacgtcatcattagattctgatgcatacctctaacggtcttgaggaaaaaaaaaaaaaaaaaaaaaaaaaaaaaaaaaaaaaaaa

SEQ ID NO: 77 - DNA, 374 bp, synthetic construct
  source 1..374: mol_type = other DNA
gactcctaatacgactcactatagggagacatactcagccaccatgcttgcattttgttattcgttgcccaatgtgggtgatgtattaaatggcaaagtatacgagaatggttatgctctatatattgatctttttgactatcctcattctgaagctatcttggcagagagtgttcagatgcatatgaatagatattttaaatatagggataaactggtagggaaaactgtaaaagttaaagtgattagagttgattatacaaaaggatatatagatgtcaattacaaaaggatgtgtagacatcaataatgcatacctctaacggtcttgaggaaaaaaaaaaaaaaaaaaaaaaaaaaaaaaaaaaaaaaaa

SEQ ID NO: 78 - DNA, 1622 bp, synthetic construct
  source 1..1622: mol_type = other DNA
gactcctaatacgactcactatagggagacatactcagccaccatggctgtgagtgatgccttgctgccatcatttagcacgttcgcatctggccccgcgggacgggaaaaaacactgagacaggccggagcgcctaacaacagatggcgggaagaactcagccacatgaagaggctccctccggtcttgcccggacgaccgtatgaccttgctgcagcaactgtagctactgatctcgagtcagggggcgccggtgccgcatgtggcgggagtaatctggcgcctcttcctagacgcgagactgaagaattcaacgatctgcttgaccttgactttatactttctaactccttgacacaccccccggaatcagtagcggcaacggtgagtagttctgcgagtgcgagcagttcaagtagtccctcatcttccggacccgcatccgccccctctacgtgttcattcacttatcccatcagggccggcaatgacccgggggttgcgccgggaggaactgggggaggcctcctctacggacgcgagtctgcaccccctcccactgccccattcaacttggcagatataaatgatgtgtctccatctgggggatttgttgcggagctcttgcgccccgaacttgatcccgtttacattccgccacaacaaccccaacctccaggcggcgggctgatgggaaaatttgtcctcaaggcgtctttgagtgctccaggtagtgaatatggttctccctctgtcatcagtgttagtaaagggtctcccgatggctcacatcctgttgtagtggctccatataacggcggaccaccacgaacctgtccaaaaatcaaacaagaagccgtcagtagttgcacgcacttgggagcgggtccacccttgtccaatgggcatcgacctgcggctcatgacttccccctcggtcgacagttgccaagccgaaccacaccgacacttgggctggaagaagtccttagttcacgggactgtcaccctgcactgccattgccaccaggtttccatccacatcccggccctaactaccctagttttctcccggaccagatgcaacctcaggtgcctccgctccactatcagggccagtcccgggggttcgtcgcgagagcaggcgaaccctgcgtctgctggccacatttcgggacccatgggatgatgctgactccgccaagctcacctcttgagctcatgccccctgggagttgtatgccagaagagcccaagccgaaacgaggtaggcgctcatggcctcgcaagaggaccgcgacgcacacatgcgactacgccggctgcggcaagacctacactaagagttcacacctgaaggcgcatcttagaacacataccggggaaaagccatatcattgcgactgggacggatgtggttggaaattcgcgcgctccgatgagttgacaagacactacagaaagcatactggacacaggcccttccagtgccagaagtgcgaccgcgctttttccagatccgatcacttggccttgcacatgaagcgacatttctaatgcatacctctaacggtcttgaggaaaaaaaaaaaaaaaaaaaaaaaaaaaaaaaaaaaaaaaa

SEQ ID NO: 79 - DNA, 737 bp, synthetic construct
  source 1..737: mol_type = other DNA
gactcctaatacgactcactatagggagacatactcagccaccatggggagtgtctcaaatcaacagttcgcaggtggatgcgcgaaggccgcagaagaagcacctgaagaagcccctgaggatgctgcgagagccgcagacgaacctcaactgctgcatggggccggaatatgtaagtggtttaacgtccgaatgggatttggctttcttagtatgaccgcaagagctggcgtcgcactcgacccaccagttgacgtcttcgtacatcagagcaagcttcacatggaaggtttccggtcacttaaggagggagaagctgtggaatttacgttcaagaaaagtgccaagggacttgaaagcatccgagttacaggtccggggggtgtcttctgtatcggtagtgaacgacggcctaaagggaagagcatgcaaaagcgcagaagcaagggtgaccgatgctacaactgcggggggctcgaccatcacgcaaaggagtgtaaacttcctccccaaccgaagaaatgtcacttttgtcagagcatatctcatatggtagcaagttgtccactcaaggcgcaacaaggtccctctgcgcaaggcaagcccacgtatttccgcgaagaggaagaggagatacactctcccaccctcctgccagaagcccagaactgatgcatacctctaacggtcttgaggaaaaaaaaaaaaaaaaaaaaaaaaaaaaaaaaaaaaaaaa

SEQ ID NO: 80 - DNA, 1061 bp, synthetic construct
  source 1..1061: mol_type = other DNA
gactcctaatacgactcactatagggagacatactcagccaccatgtacaatatgatggagacagaactgaaaccacctgggccgcagcaaacaagcggtggcggaggtggaaactctacggctgcggctgctggaggaaaccagaagaatagtccggatagggttaagcggcctatgaacgcgtttatggtgtggtccagaggtcaacgccgcaagatggcacaggaaaacccaaagatgcacaactcagagatatccaagcgcttgggtgctgagtggaagcttctctctgagacggagaagaggccatttatcgatgaggctaagcgactgcgcgccttgcacatgaaggaacaccccgactacaagtataggccacgccgcaaaactaaaacccttatgaagaaagataagtacaccctgccaggagggcttttggcccccggtggaaactcaatggcgagcggggtgggcgtcggagcgggacttggagcgggtgtaaatcagaggatggactcatacgcgcacatgaatgggtggtcaaatggttcctattccatgatgcaggatcaattgggttatcctcaacatccggggcttaatgcccatggagcagcccagatgcaaccaatgcataggtatgatgtgtccgcacttcaatataactcaatgactagctcccaaacgtacatgaacggttcaccgacttattctatgtcttacagccagcaaggaacccctggcatggctctcggaagcatgggctcagttgttaaatctgaagctagctcaagcccaccagttgtaacctccagtagccactcccgagcgccctgccaggcaggcgacttgcgcgacatgatatcaatgtatctcccaggcgcagaagtacctgaaccggctgcgccttcccgattgcacatgtcacagcattaccaatccggtccggtgccaggcactgcaataaatgggacccttcctctgtcacacatgtgatgcatacctctaacggtcttgaggaaaaaaaaaaaaaaaaaaaaaaaaaaaaaaaaaaaaaaaa

SEQ ID NO: 81 - DNA, 441 bp, synthetic construct
  source 1..441: mol_type = other DNA
gctttatttgtaaccattataagctgcaataaacaagttaacaacaacaattgcattcattttatgtttcaggttcagggggaggtgtgggaggttttttaaagcaagtaaaacctctacaaatgtggtatggctgattatgatcatgaacagactgtgaggactgaggggcctgaaatgagccttgggactgtgaatcaatgcctgtttcatgccctgagtcttccatgttcttctccccaccatcttcatttttatcagcattttcctggctgtcttcatcatcatcatcactgtttcttagccaatctaaaactccagttcccatagccacattaaacttcattttttgatacactgacaaactaaactctttgtccaatctctctttccactccacaattctgctctgaatactttgagcaaactcagccacaggtc

SEQ ID NO: 82 - RNA, 804 nt, synthetic construct
  source 1..804: mol_type = other RNA
gggagacatactcagccaccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaatgcatacctctaacggtcttgaggaaaaaaaaaaaaaaaaaaaaaaaaaaaaaaaaaaaaaaaa

SEQ ID NO: 83 - RNA, 684 nt, synthetic construct
  source 1..684: mol_type = other RNA
gggagacatactcagccaccatgaccgaatataagccgactgttagactcgcgactcgcgacgacgttccgagagctgtaaggacgctcgcggctgcgttcgcggactatcccgccactaggcacactgtagacccggaccgacacatagagagagtgacggaactccaagagttgtttttgacgagggttgggctcgatattgggaaggtctgggttgcggacgacggtgcagcagtggctgtgtggaccacaccggagagtgtcgaagcgggagctgttttcgcagaaataggaccgaggatggctgaattgtcaggatcccggttggcagcgcaacaacagatggaaggcctccttgccccacacagaccaaaagagcccgcatggttccttgccacggtaggtgtttccccagatcaccaaggaaaaggccttgggtcagctgttgtattgcctggggtagaagccgccgagcgcgcaggagttccagcttttcttgaaacgtctgcgcctcgcaatctccccttctacgagcgactgggatttacggtgactgcagatgtagaggttcctgagggtccccgaacatggtgcatgacgcgaaaaccgggtgcatagtgcatacctctaacggtcttgaggaaaaaaaaaaaaaaaaaaaaaaaaaaaaaaaaaaaaaaaa

SEQ ID NO: 84 - RNA, 1167 nt, synthetic construct
  source 1..1167: mol_type = other RNA
gggagacatactcagccaccatggcgggacacctggcttcggatttcgccttctcgccccctccaggtggtggaggtgatgggccaggggggccggagccgggctgggttgatcctcggacctggctaagcttccaaggccctcctggagggccaggaatcgggccgggggttgggccaggctctgaggtgtgggggattcccccatgccccccgccgtatgagttctgtggggggatggcgtactgtgggccccaggttggagtggggctagtgccccaaggcggcttggagacctctcagcctgagggcgaagcaggagtcggggtggagagcaactccgatggggcctccccggagccctgcaccgtcacccctggtgccgtgaagctggagaaggagaagctggagcaaaacccggaggagtcccaggacatcaaagctctgcagaaagaactcgagcaatttgccaagctcctgaagcagaagaggatcaccctgggatatacacaggccgatgtggggctcaccctgggggttctatttgggaaggtattcagccaaacgaccatctgccgctttgaggctctgcagcttagcttcaagaacatgtgtaagctgcggcccttgctgcagaagtgggtggaggaagctgacaacaatgaaaatcttcaggagatatgcaaagcagaaaccctcgtgcaggcccgaaagagaaagcgaaccagtatcgagaaccgagtgagaggcaacctggagaatttgttcctgcagtgcccgaaacccacactgcagcagatcagccacatcgcccagcagcttgggctcgagaaggatgtggtccgagtgtggttctgtaaccggcgccagaagggcaagcgatcaagcagcgactatgcacaacgagaggattttgaggctgctgggtctcctttctcagggggaccagtgtcctttcctctggccccagggccccattttggtaccccaggctatgggagccctcacttcactgcactgtactcctcggtccctttccctgagggggaagcctttccccctgtctccgtcaccactctgggctctcccatgcattcaaactgatgcatacctctaacggtcttgaggaaaaaaaaaaaaaaaaaaaaaaaaaaaaaaaaaaaaaaaa

SEQ ID NO: 85 - RNA, 1002 nt, synthetic construct
  source 1..1002: mol_type = other RNA
gggagacatactcagccaccatgagtgtggatccagcttgtccccaaagcttgccttgctttgaagcatccgactgtaaagaatcttcacctatgcctgtgatttgtgggcctgaagaaaactatccatccttgcaaatgtcttctgctgagatgcctcacacggagactgtctctcctcttccttcctccatggatctgcttattcaggacagccctgattcttccaccagtcccaaaggcaaacaacccacttctgcagagaagagtgtcgcaaaaaaggaagacaaggtcccggtcaagaaacagaagaccagaactgtgttctcttccacccagctgtgtgtactcaatgatagatttcagagacagaaatacctcagcctccagcagatgcaagaactctccaacatcctgaacctcagctacaaacaggtgaagacctggttccagaaccagagaatgaaatctaagaggtggcagaaaaacaactggccgaagaatagcaatggtgtgacgcagaaggcctcagcacctacctaccccagcctttactcttcctaccaccagggatgcctggtgaacccgactgggaaccttccaatgtggagcaaccagacctggaacaattcaacctggagcaaccagacccagaacatccagtcctggagcaaccactcctggaacactcagacctggtgcacccaatcctggaacaatcaggcctggaacagtcccttctataactgtggagaggaatctctgcagtcctgcatgcagttccagccaaattctcctgccagtgacttggaggctgccttggaagctgctggggaaggccttaatgtaatacagcagaccactaggtattttagtactccacaaaccatggatttattcctaaactactccatgaacatgcaacctgaagacgtgtgatgcatacctctaacggtcttgaggaaaaaaaaaaaaaaaaaaaaaaaaaaaaaaaaaaaaaaaa

SEQ ID NO: 86 - RNA, 1038 nt, synthetic construct
  source 1..1038: mol_type = other RNA
gggagacatactcagccaccatgtacaacatgatggagacggagctgaagccgccgggcccgcagcaaacttcggggggcggcggcggcaactccaccgcggcggcggccggcggcaaccagaaaaacagcccggaccgcgtcaagcggcccatgaatgccttcatggtgtggtcccgcgggcagcggcgcaagatggcccaggagaaccccaagatgcacaactcggagatcagcaagcgcctgggcgccgagtggaaacttttgtcggagacggagaagcggccgttcatcgacgaggctaagcggctgcgagcgctgcacatgaaggagcacccggattataaataccggccccggcggaaaaccaagacgctcatgaagaaggataagtacacgctgcccggcgggctgctggcccccggcggcaatagcatggcgagcggggtcggggtgggcgccggcctgggcgcgggcgtgaaccagcgcatggacagttacgcgcacatgaacggctggagcaacggcagctacagcatgatgcaggaccagctgggctacccgcagcacccgggcctcaatgcgcacggcgcagcgcagatgcagcccatgcaccgctacgacgtgagcgccctgcagtacaactccatgaccagctcgcagacctacatgaacggctcgcccacctacagcatgtcctactcgcagcagggcacccctggcatggctcttggctccatgggttcggtggtcaagtccgaggccagctccagcccccctgtggttacctcttcctcccactccagggcgccctgccaggccggggacctccgggacatgatcagcatgtatctccccggcgccgaggtgccggaacccgccgcccccagcagacttcacatgtcccagcactaccagagcggcccggtgcccggcacggccattaacggcacactgcccctctcacacatgtgatgcatacctctaacggtcttgaggaaaaaaaaaaaaaaaaaaaaaaaaaaaaaaaaaaaaaaaa

SEQ ID NO: 87 - RNA, 1404 nt, synthetic construct
  source 1..1404: mol_type = other RNA
gggagacatactcagccaccatgcccctcaacgttagcttcaccaacaggaactatgacctcgactacgactcggtgcagccgtatttctactgcgacgaggaggagaacttctaccagcagcagcagcagagcgagctgcagcccccggcgcccagcgaggatatctggaagaaattcgagctgctgcccgccccgcccctgtcccctagccgccgctccgggctctgctcgccctcctacgttgcggtcacacccttctcccttcggggagacaacgacggcggtggcgggagcttctccacggccgaccagctggagatggtgaccgagctgctgggaggagacatggtgaaccagagtttcatctgcgacccggacgacgagaccttcatcaaaaacatcatcatccaggactgtatgtggagcggcttctcggccgccgccaagctcgtctcagagaagctggcctcctaccaggctgcgcgcaaagacagcggcagcccgaaccccgcccgcggccacagcgtctgctccacctccagcttgtacctgcaggatctgagcgccgccgcctcagagtgcatcgacccctcggtggtcttcccctaccctctcaacgacagcagctcgcccaagtcctgcgcctcgcaagactccagcgccttctctccgtcctcggattctctgctctcctcgacggagtcctccccgcagggcagccccgagcccctggtgctccatgaggagacaccgcccaccaccagcagcgactctgaggaggaacaagaagatgaggaagaaatcgatgttgtttctgtggaaaagaggcaggctcctggcaaaaggtcagagtctggatcaccttctgctggaggccacagcaaacctcctcacagcccactggtcctcaagaggtgccacgtctccacacatcagcacaactacgcagcgcctccctccactcggaaggactatcctgctgccaagagggtcaagttggacagtgtcagagtcctgagacagatcagcaacaaccgaaaatgcaccagccccaggtcctcggacaccgaggagaatgtcaagaggcgaacacacaacgtcttggagcgccagaggaggaacgagctaaaacggagcttttttgccctgcgtgaccagatcccggagttggaaaacaatgaaaaggcccccaaggtagttatccttaaaaaagccacagcatacatcctgtccgtccaagcagaggagcaaaagctcatttctgaagaggacttgttgcggaaacgacgagaacagttgaaacacaaacttgaacagctacggaactcttgtgcgtaatgcatacctctaacggtcttgaggaaaaaaaaaaaaaaaaaaaaaaaaaaaaaaaaaaaaaaaa

SEQ ID NO: 88 - RNA, 714 nt, synthetic construct
  source 1..714: mol_type = other RNA
gggagacatactcagccaccatgggctccgtgtccaaccagcagtttgcaggtggctgcgccaaggcggcagaagaggcgcccgaggaggcgccggaggacgcggcccgggcggcggacgagcctcagctgctgcacggtgcgggcatctgtaagtggttcaacgtgcgcatggggttcggcttcctgtccatgaccgcccgcgccggggtcgcgctcgaccccccagtggatgtctttgtgcaccagagtaagctgcacatggaagggttccggagcttgaaggagggtgaggcagtggagttcacctttaagaagtcagccaagggtctggaatccatccgtgtcaccggacctggtggagtattctgtattgggagtgagaggcggccaaaaggaaagagcatgcagaagcgcagatcaaaaggagacaggtgctacaactgtggaggtctagatcatcatgccaaggaatgcaagctgccaccccagcccaagaagtgccacttctgccagagcatcagccatatggtagcctcatgtccgctgaaggcccagcagggccctagtgcacagggaaagccaacctactttcgagaggaagaagaagaaatccacagccctaccctgctcccggaggcacagaattgatgcatacctctaacggtcttgaggaaaaaaaaaaaaaaaaaaaaaaaaaaaaaaaaaaaaaaaa

SEQ ID NO: 89 - RNA, 1599 nt, synthetic construct
  source 1..1599: mol_type = other RNA
gggagacatactcagccaccatggctgtcagcgacgcgctgctcccatctttctccacgttcgcgtctggcccggcgggaagggagaagacactgcgtcaagcaggtgccccgaataaccgctggcgggaggagctctcccacatgaagcgacttcccccagtgcttcccggccgcccctatgacctggcggcggcgaccgtggccacagacctggagagcggcggagccggtgcggcttgcggcggtagcaacctggcgcccctacctcggagagagaccgaggagttcaacgatctcctggacctggactttattctctccaattcgctgacccatcctccggagtcagtggccgccaccgtgtcctcgtcagcgtcagcctcctcttcgtcgtcgccgtcgagcagcggccctgccagcgcgccctccacctgcagcttcacctatccgatccgggccgggaacgacccgggcgtggcgccgggcggcacgggcggaggcctcctctatggcagggagtccgctccccctccgacggctcccttcaacctggcggacatcaacgacgtgagcccctcgggcggcttcgtggccgagctcctgcggccagaattggacccggtgtacattccgccgcagcagccgcagccgccaggtggcgggctgatgggcaagttcgtgctgaaggcgtcgctgagcgcccctggcagcgagtacggcagcccgtcggtcatcagcgtcagcaaaggcagccctgacggcagccacccggtggtggtggcgccctacaacggcgggccgccgcgcacgtgccccaagatcaagcaggaggcggtctcttcgtgcacccacttgggcgctggaccccctctcagcaatggccaccggccggctgcacacgacttccccctggggcggcagctccccagcaggactaccccgaccctgggtcttgaggaagtgctgagcagcagggactgtcaccctgccctgccgcttcctcccggcttccatccccacccggggcccaattacccatccttcctgcccgatcagatgcagccgcaagtcccgccgctccattaccaaggtcagtcccggggatttgtagctcgggctggggagccctgtgtgtgctggccccacttcgggacacacgggatgatgctcaccccaccttcttcacccctagagctcatgccacccggttcctgcatgccagaggagcccaagccaaagaggggaagacgatcgtggccccggaaaaggaccgccacccacacttgtgattacgcgggctgcggcaaaacctacacaaagagttcccatctcaaggcacacctgcgaacccacacaggtgagaaaccttaccactgtgactgggacggctgtggatggaaattcgcccgctcagatgaactgaccaggcactaccgtaaacacacggggcaccgcccgttccagtgccaaaaatgcgaccgagcattttccaggtcggaccacctcgccttacacatgaagaggcatttttaatgcatacctctaacggtcttgaggaaaaaaaaaaaaaaaaaaaaaaaaaaaaaaaaaaaaaaaa

SEQ ID NO: 90 - RNA, 2211 nt, synthetic construct
  source 1..2211: mol_type = other RNA
gggagacatactcagccaccatggataaagttttaaacagagaggaatctttgcagctaatggaccttctaggtcttgaaaggagtgcctgggggaatattcctctgatgagaaaggcatatttaaaaaaatgcaaggagtttcatcctgataaaggaggagatgaagaaaaaatgaagaaaatgaatactctgtacaagaaaatggaagatggagtaaaatatgctcatcaacctgactttggaggcttctgggatgcaactgagattccaacctatggaactgatgaatgggagcagtggtggaatgcctttaatgaggaaaacctgttttgctcagaagaaatgccatctagtgatgatgaggctactgctgactctcaacattctactcctccaaaaaagaagagaaaggtagaagaccccaaggactttccttcagaattgctaagttttttgagtcatgctgtgtttagtaatagaactcttgcttgctttgctatttacaccacaaaggaaaaagctgcactgctatacaagaaaattatggaaaaatattctgtaacctttataagtaggcataacagttataatcataacatactgttttttcttactccacacaggcatagagtgtctgctattaataactatgctcaaaaattgtgtacctttagctttttaatttgtaaaggggttaataaggaatatttgatgtatagtgccttgactagagatccattttctgttattgaggaaagtttgccaggtgggttaaaggagcatgattttaatccagaagaagcagaggaaactaaacaagtgtcctggaagcttgtaacagagtatgcaatggaaacaaaatgtgatgatgtgttgttattgcttgggatgtacttggaatttcagtacagttttgaaatgtgtttaaaatgtattaaaaaagaacagcccagccactataagtaccatgaaaagcattatgcaaatgctgctatatttgctgacagcaaaaaccaaaaaaccatatgccaacaggctgttgatactgttttagctaaaaagcgggttgatagcctacaattaactagagaacaaatgttaacaaacagatttaatgatcttttggataggatggatataatgtttggttctacaggctctgctgacatagaagaatggatggctggagttgcttggctacactgtttgttgcccaaaatggattcagtggtgtatgactttttaaaatgcatggtgtacaacattcctaaaaaaagatactggctgtttaaaggaccaattgatagtggtaaaactacattagcagctgctttgcttgaattatgtggggggaaagctttaaatgttaatttgcccttggacaggctgaactttgagctaggagtagctattgaccagtttttagtagtttttgaggatgtaaagggcactggaggggagtccagagatttgccttcaggtcagggaattaataacctggacaatttaagggattatttggatggcagtgttaaggtaaacttagaaaagaaacacctaaataaaagaactcaaatatttccccctggaatagtcaccatgaatgagtacagtgtgcctaaaacactgcaggccagatttgtaaaacaaatagattttaggcccaaagattatttaaagcattgcctggaacgcagtgagtttttgttagaaaagagaataattcaaagtggcattgctttgcttcttatgttaatttggtacagacctgtggctgagtttgctcaaagtattcagagcagaattgtggagtggaaagagagattggacaaagagtttagtttgtcagtgtatcaaaaaatgaagtttaatgtggctatgggaattggagttttagattggctaagaaacagtgatgatgatgatgaagacagccaggaaaatgctgataaaaatgaagatggtggggagaagaacatggaagactcagggcatgaaacaggcattgattcacagtcccaaggctcatttcaggcccctcagtcctcacagtctgttcatgatcataatcagccataccacatttgtagaggttttacttgctttaaaaaacctcccacacctccccctgaacctgaaacataatgcatacctctaacggtcttgaggaaaaaaaaaaaaaaaaaaaaaaaaaaaaaaaaaaaaaaaa

SEQ ID NO: 91 - RNA, 1143 nt, synthetic construct
  source 1..1143: mol_type = other RNA
gggagacatactcagccaccatgggcacgatgaaaatgatggtacatatatatttcgtatcattattgttattgctattccacagttacgccatagacatcgaaaatgaaatcacagaattcttcaataaaatgagagatactctaccagctaaagactctaaatggttgaatccagcatgtatgttcggaggcacaatgaatgatatagccgctctaggagagccattcagcgcaaagtgtcctcctattgaagacagtcttttatcgcacagatataaagactatgtggttaaatgggaaaggctagaaaaaaatagacggcgacaggtttctaataaacgtgttaaacatggtgatttatggatagccaactatacatctaaattcagtaaccgtaggtatttgtgcaccgtaactacaaagaatggtgactgtgttcagggtatagttagatctcatattagaaaacctccttcatgcattccaaaaacatatgaactaggtactcatgataagtatggcatagacttatactgtggaattctttacgcaaaacattataataatataacttggtataaagataataaggaaattaatatcgacgacattaagtattcacaaacgggaaaggaattaattattcataatccagagttagaagatagcggaagatacgactgttacgttcattacgacgacgttagaatcaagaatgatatcgtagtatcaagatgtaaaatacttacggttataccgtcacaagaccacaggtttaaactaatactagatccaaaaatcaacgtaacgataggagaacctgccaatataacatgcactgctgtgtcaacgtcattattgattgacgatgtactgattgaatgggaaaatccatccggatggcttataggattcgattttgatgtatactctgttttaactagtagaggcggtattaccgaggcgaccttgtactttgaaaatgttactgaagaatatataggtaatacatataaatgtcgtggacacaactattattttgaaaaaacccttacaactacagtagtattggagtaatgcatacctctaacggtcttgaggaaaaaaaaaaaaaaaaaaaaaaaaaaaaaaaaaaaaaaaa

SEQ ID NO: 92 - RNA, 657 nt, synthetic construct
  source 1..657: mol_type = other RNA
gggagacatactcagccaccatgtctaaaatctatatcgacgagcgttctaacgcagagattgtgtgtgaggctattaaaaccattggaatcgaaggagctactgctgcacaactaactagacaacttaatatggagaagcgagaagttaataaagctctgtacgatcttcaacgtagtgctatggtgtacagctccgacgatattcctcctcgttggtttatgacaacggaggcggataagccggatgctgatgctatggctgacgtcataatagatgatgtatcccgcgaaaaatcaatgagagaggatcataagtcttttgatgatgttattccggctaaaaaaattattgattggaaaggtgctaaccctgtcaccgttattaatgagtactgccaaattactaggagagattggtcttttcgtattgaatcagtggggcctagtaactctcctacattttatgcctgtgtagacatcgacggaagagtattcgataaggcagatggaaaatctaaacgagatgctaaaaataatgcagctaaattggcagtagataaacttcttggttacgtcatcattagattctgatgcatacctctaacggtcttgaggaaaaaaaaaaaaaaaaaaaaaaaaaaaaaaaaaaaaaaaa

SEQ ID NO: 93 - RNA, 351 nt, synthetic construct
  source 1..351: mol_type = other RNA
gggagacatactcagccaccatgcttgcattttgttattcgttgcccaatgtgggtgatgtattaaatggcaaagtatacgagaatggttatgctctatatattgatctttttgactatcctcattctgaagctatcttggcagagagtgttcagatgcatatgaatagatattttaaatatagggataaactggtagggaaaactgtaaaagttaaagtgattagagttgattatacaaaaggatatatagatgtcaattacaaaaggatgtgtagacatcaataatgcatacctctaacggtcttgaggaaaaaaaaaaaaaaaaaaaaaaaaaaaaaaaaaaaaaaaa

SEQ ID NO: 94 - protein, 360 aa, synthetic construct
MAGHLASDFAFSPPPGGGGDGPGGPEPGWVDPRTWLSFQGPPGGPGIGPGVGPGSEVWGIPPCPPPYEFCGGMAYCGPQVGVGLVPQGGLETSQPEGEAGVGVESNSDGASPEPCTVTPGAVKLEKEKLEQNPEESQDIKALQKELEQFAKLLKQKRITLGYTQADVGLTLGVLFGKVFSQTTICRFEALQLSFKNMCKLRPLLQKWVEEADNNENLQEICKAETLVQARKRKRTSIENRVRGNLENLFLQCPKPTLQQISHIAQQLGLEKDVVRVWFCNRRQKGKRSSSDYAQREDFEAAGSPFSGGPVSFPLAPGPHFGTPGYGSPHFTALYSSVPFPEGEAFPPVSVTTLGSPMHSN

SEQ ID NO: 95 - protein, 304 aa, synthetic construct
MSVDPACPQSLPCFEASDCKESSPMPVICGPEENYPSLQMSSAEMPHTETVSPLPSSMDLLIQDSPDSSTSPKGKQPTSAEKSVAKKEDKVPVKKQKTRTVFSSTQLCVLNDRFQRQKYLSLQQMQELSNILNLSYKQVKTWFQNQRMKSKRWQKNNWPKNSNGVTQKASAPTYPSLYSSYHQGCLVNPTGNLPMWSNQTWNNSTWSNQTQNIQSWSNHSWNTQTWCTQSWNNQAWNSPFYNCGEESLQSCMQFQPNSPASDLEAALEAAGEGLNVIQQTTRYFSTPTMDLFLNYSMNMQPEDV

SEQ ID NO: 96 - protein, 439 aa, synthetic construct
MPLNVSFTNRNYDLDYDSVQPYFYCDEEENFYQQQQQSELQPPAPSEDIWKKFELLPAPPLSPSRRSGLCSPSYVAVTPFSLRGDNDGGGGSFSTADQLEMVTELLGGDMVNQSFICDPDDETFIKNIIIQDCMWSGFSAAAKLVSEKLASYQAARKDSGSPNPARGHSVCSTSSLYLQDLSAAASECIDPSVVFPYPLNDSSSPKSCASQDSSAFSPSSDSLLSSTESSPQGSPEPLVLHEETPPTTSSDSEEEQEDEEEIDVVSVEKRQAPGKRSESGSPSAGGHSKPPHSPLVLKRCHVSTHQHNYAAPPSTRKDYPAAKRVKLDSVRVLRQISNNRKCTSPRSSDTEENVKRRTHNVLERQRRNELKRSFFALRDQIPELENNEKAPKVVILKKATAYILSVQAEEQKLISEEDLLRKRREQLKHKLEQLRNSCA

SEQ ID NO: 97 - protein, 708 aa, synthetic construct
MDKVLNREESLQLMDLLGLERSAWGNIPLMRKAYLKKCKEFHPDKGGDEEKMKKMNTLYKKMEDGVKYAHQPDFGGFWDATEIPTYGTDEWEQWWNAFNEENLFCSEEMPSSDDEATADSQHSTPPKKKRKVEDPKDFPSELLSFLSHAVFSNRTLACFAIYTTKEKAALLYKKIMEKYSVTFISRHNSYNHNILFFLTPHRHRVSAINNYAQKLCTFSFLICKGVNKEYLMYSALTRDPFSVIEESLPGGLKEHDFNPEEAEETKQVSWKLVTEYAMETKCDDVLLLLGMYLEFQYSFEMCLKCIKKEQPSHYKYHEKHYANAAIFADSKNQKTICQQAVDTVLAKKRVDSLQLTREQMLTNRFNDLLDRMDIMFGSTGSADIEEWMAGVAWLHCLLPKMDSVVYDFLKCMVYNIPKKRYWLFKGPIDSGKTTLAAALLELCGGKALNVNLPLDRLNFELGVAIDQFLVVFEDVKGTGGESRDLPSGQGINNLDNLRDYLDGSVKVNLEKKHLNKRTQIFPPGIVTMNEYSVPKTLQARFVKQIDFRPKDYLKHCLERSEFLLEKRIIQSGIALLLMLIWYRPVAEFAQSIQSRIVEWKERLDKEFSLSVYQKMKFNVAMGIGVLDWLRNSDDDDEDSQENADKNEDGGEKNMEDSGHETGIDSQSQGSFQAPQSSQSVHDHNQPYHICRGFTCFKKPPTPPPEPET

SEQ ID NO: 98 - protein, 352 aa, synthetic construct
MGTMKMMVHIYFVSLLLLLFHSYAIDIENEITEFFNKMRDTLPAKDSKWLNPACMFGGTMNDIAALGEPFSAKCPPIEDSLLSHRYKDYVVKWERLEKNRRRQVSNKRVKHGDLWIANYTSKFSNRRYLCTVTTKNGDCVQGIVRSHIRKPPSCIPKTYELGTHDKYGIDLYCGILYAKHYNNITWYKDNKEINIDDIKYSQTGKELIIHNPELEDSGRYDCYVHYDDVRIKNDIVVSRCKILTVIPSQDHRFKLILDPKINVTIGEPANITCTAVSTSLLIDDVLIEWENPSGWLIGFDFDVYSVLTSRGGITEATLYFENVTEEYIGNTYKCRGHNYYFEKTLTTTVVLE

SEQ ID NO: 99 - protein, 190 aa, synthetic construct
MSKIYIDERSNAEIVCEAIKTIGIEGATAAQLTRQLNMEKREVNKALYDLQRSAMVYSSDDIPPRWFMTTEADKPDADAMADVIIDDVSREKSMREDHKSFDDVIPAKKIIDWKGANPVTVINEYCQITRRDWSFRIESVGPSNSPTFYACVDIDGRVFDKADGKSKRDAKNNAAKLAVDKLLGYVIIRF

SEQ ID NO: 100 - protein, 88 aa, synthetic construct
MLAFCYSLPNVGDVLNGKVYENGYALYIDLFDYPHSEAILAESVQMHMNRYFKYRDKLVGKTVKVKVIRVDYTKGYIDVNYKRMCRHQ

SEQ ID NO: 101 - protein, 504 aa, synthetic construct
MAVSDALLPSFSTFASGPAGREKTLRQAGAPNNRWREELSHMKRLPPVLPGRPYDLAAATVATDLESGGAGAACGGSNLAPLPRRETEEFNDLLDLDFILSNSLTHPPESVAATVSSSASASSSSSPSSSGPASAPSTCSFTYPIRAGNDPGVAPGGTGGGLLYGRESAPPPTAPFNLADINDVSPSGGFVAELLRPELDPVYIPPQQPQPPGGGLMGKFVLKASLSAPGSEYGSPSVISVSKGSPDGSHPVVVAPYNGGPPRTCPKIKQEAVSSCTHLGAGPPLSNGHRPAAHDFPLGRQLPSRTTPTLGLEEVLSSRDCHPALPLPPGFHPHPGPNYPSFLPDQMQPQVPPLHYQGQSRGFVARAGEPCVCWPHFGTHGMMLTPPSSPLELMPPGSCMPEEPKPKRGRRSWPRKRTATHTCDYAGCGKTYTKSSHLKAHLRTHTGEKPYHCDWDGCGWKFARSDELTRHYRKHTGHRPFQCQKCDRAFSRSDHLALHMKRHF

SEQ ID NO: 102 - protein, 209 aa, synthetic construct
MGSVSNQQFAGGCAKAAEEAPEEAPEDAARAADEPQLLHGAGICKWFNVRMGFGFLSMTARAGVALDPPVDVFVHQSKLHMEGFRSLKEGEAVEFTFKKSAKGLESIRVTGPGGVFCIGSERRPKGKSMQKRRSKGDRCYNCGGLDHHAKECKLPPQPKKCHFCQSISHMVASCPLKAQQGPSAQGKPTYFREEEEEIHSPTLLPEAQN

SEQ ID NO: 103 - protein, 317 aa, synthetic construct
MYNMMETELKPPGPQQTSGGGGGNSTAAAAGGNQKNSPDRVKRPMNAFMVWSRGQRRKMAQENPKMHNSEISKRLGAEWKLLSETEKRPFIDEAKRLRALHMKEHPDYKYRPRRKTKTLMKKDKYTLPGGLLAPGGNSMASGVGVGAGLGAGVNQRMDSYAHMNGWSNGSYSMMQDQLGYPQHPGLNAHGAAQMQPMHRYDVSALQYNSMTSSQTYMNGSPTYSMSYSQQGTPGMALGSMGSVVKSEASSSPPVVTSSSHSRAPCQAGDLRDMISMYLPGAEVPEPAAPSRLHMSQHYQSGPVPGTAINGTLPLSHM

SEQ ID NO: 104 - skipped

SEQ ID NO: 105 - RNA, 1599 nt, synthetic construct
  source 1..1599: mol_type = other RNA
gggagacatactcagccaccatggctgtgagtgatgccttgctgccatcatttagcacgttcgcatctggccccgcgggacgggaaaaaacactgagacaggccggagcgcctaacaacagatggcgggaagaactcagccacatgaagaggctccctccggtcttgcccggacgaccgtatgaccttgctgcagcaactgtagctactgatctcgagtcagggggcgccggtgccgcatgtggcgggagtaatctggcgcctcttcctagacgcgagactgaagaattcaacgatctgcttgaccttgactttatactttctaactccttgacacaccccccggaatcagtagcggcaacggtgagtagttctgcgagtgcgagcagttcaagtagtccctcatcttccggacccgcatccgccccctctacgtgttcattcacttatcccatcagggccggcaatgacccgggggttgcgccgggaggaactgggggaggcctcctctacggacgcgagtctgcaccccctcccactgccccattcaacttggcagatataaatgatgtgtctccatctgggggatttgttgcggagctcttgcgccccgaacttgatcccgtttacattccgccacaacaaccccaacctccaggcggcgggctgatgggaaaatttgtcctcaaggcgtctttgagtgctccaggtagtgaatatggttctccctctgtcatcagtgttagtaaagggtctcccgatggctcacatcctgttgtagtggctccatataacggcggaccaccacgaacctgtccaaaaatcaaacaagaagccgtcagtagttgcacgcacttgggagcgggtccacccttgtccaatgggcatcgacctgcggctcatgacttccccctcggtcgacagttgccaagccgaaccacaccgacacttgggctggaagaagtccttagttcacgggactgtcaccctgcactgccattgccaccaggtttccatccacatcccggccctaactaccctagttttctcccggaccagatgcaacctcaggtgcctccgctccactatcagggccagtcccgggggttcgtcgcgagagcaggcgaaccctgcgtctgctggccacatttcgggacccatgggatgatgctgactccgccaagctcacctcttgagctcatgccccctgggagttgtatgccagaagagcccaagccgaaacgaggtaggcgctcatggcctcgcaagaggaccgcgacgcacacatgcgactacgccggctgcggcaagacctacactaagagttcacacctgaaggcgcatcttagaacacataccggggaaaagccatatcattgcgactgggacggatgtggttggaaattcgcgcgctccgatgagttgacaagacactacagaaagcatactggacacaggcccttccagtgccagaagtgcgaccgcgctttttccagatccgatcacttggccttgcacatgaagcgacatttctaatgcatacctctaacggtcttgaggaaaaaaaaaaaaaaaaaaaaaaaaaaaaaaaaaaaaaaaa

SEQ ID NO: 106 - RNA, 714 nt, synthetic construct
  source 1..714: mol_type = other RNA
gggagacatactcagccaccatggggagtgtctcaaatcaacagttcgcaggtggatgcgcgaaggccgcagaagaagcacctgaagaagcccctgaggatgctgcgagagccgcagacgaacctcaactgctgcatggggccggaatatgtaagtggtttaacgtccgaatgggatttggctttcttagtatgaccgcaagagctggcgtcgcactcgacccaccagttgacgtcttcgtacatcagagcaagcttcacatggaaggtttccggtcacttaaggagggagaagctgtggaatttacgttcaagaaaagtgccaagggacttgaaagcatccgagttacaggtccggggggtgtcttctgtatcggtagtgaacgacggcctaaagggaagagcatgcaaaagcgcagaagcaagggtgaccgatgctacaactgcggggggctcgaccatcacgcaaaggagtgtaaacttcctccccaaccgaagaaatgtcacttttgtcagagcatatctcatatggtagcaagttgtccactcaaggcgcaacaaggtccctctgcgcaaggcaagcccacgtatttccgcgaagaggaagaggagatacactctcccaccctcctgccagaagcccagaactgatgcatacctctaacggtcttgaggaaaaaaaaaaaaaaaaaaaaaaaaaaaaaaaaaaaaaaaa

SEQ ID NO: 107 - RNA, 1037 nt, synthetic construct
  source 1..1037: mol_type = other RNA
gggagacatactcagccaccatgtacaatatgatggagacagaactgaaaccacctgggccgcagcaaacaagcggtggcggaggtggaaactctacggctgcggctgctggaggaaaccagaagaatagtccggatagggttaagcggcctatgaacgcgtttatggtgtggtccagaggtcaacgccgcaagatggcacaggaaaacccaaagatgcacaactcagagatatccaagcgcttgggtgctgagtggaagcttctctctgagacggagaagaggccatttatcgatgaggctaagcgactgcgcgccttgcacatgaaggaacaccccgactacaagtataggccacgccgcaaaactaaaacccttatgaagaaagataagtacaccctgccaggagggcttttggcccccggtggaaactcaatggcgagcggggtgggcgtcggagcgggacttggagcgggtgtaaatcagaggatggactcatacgcgcacatgaatgggtggtcaaatggttcctattccatgatgcaggatcaattgggttatcctcaacatccggggcttaatgcccatggagcagcccagatgcaaccaatgcataggtatgatgtgtccgcacttcaatataactcaatgactagctcccaaacgtacatgaacggttcaccgacttattctatgtcttacagccagcaaggaacccctggcatggctctcggaagcatgggctcagttgttaaatctgaagctagctcaagcccaccagttgtaacctccagtagccactcccgagcgccctgccaggcaggcgacttgcgcgacatgatatcaatgtatctcccaggcgcagaagtacctgaaccggctgcgccttcccgattgcacatgtcacagcattaccaatccggtccggtgccaggcactgcaataaatgggacccttcctctgtcacacatgtgatgcatacctctaacggtcttgaggaaaaaaaaaaaaaaaaaaaaaaaaaaaaaaaaaaaaaaa